(19) United States
(12) Patent Application Publication
(10) Pub. No.: US 2024/0025839 A1
(43) Pub. Date: Jan. 25, 2024

(54) METHOD OF SYNTHESIS

(71) Applicant: University of Durham, Durham (GB)

(72) Inventors: Matthew Oliver Kitching, Durham (GB); Mark Patrick Walsh, Durham (GB)

(21) Appl. No.: 18/036,515

(22) PCT Filed: Nov. 11, 2021
(86) PCT No.: PCT/GB2021/052914

§ 371 Date: May 11, 2023

(30) Foreign Application Priority Data
Nov. 11, 2020 (GB) 2017799.4

(51) Int. Cl. C07C 209/12

(52) U.S. Cl. CPC C07C 209/12; C07B 2200/07

(57) ABSTRACT

The present invention provides a method of making an enantiomerically enriched tertiary or quaternary ammonium salt, and the use of a non-racemic chiral compound in the synthesis of an enantiomerically enriched tertiary or quaternary ammonium salt. The control of nitrogen-based chirality, achieved via the method of the invention, is useful where a specific tertiary or quaternary ammonium enantiomer is preferred over the other enantiomer, for example where a specific tertiary or quaternary ammonium enantiomer is more effective than the other enantiomer in treating a specific medical condition.

### FIELD OF THE INVENTION

The present invention provides a method of making an enantiomerically enriched tertiary or quaternary ammonium salt and the use of a non-racemic chiral compound in the synthesis of an enantiomerically enriched tertiary or quaternary ammonium salt. The control of nitrogen-based chirality, achieved via the method of the invention, is useful where a specific tertiary or quaternary ammonium enantiomer is preferred over the other enantiomer, for example where a specific tertiary or quaternary ammonium enantiomer is more effective than the other enantiomer in treating a specific medical condition.

### BACKGROUND OF THE INVENTION

Chirality is a property of molecules that do not possess an internal plane or point of symmetry and that may exist in one of two distinguishable, non-superposable mirror image forms—the R-(rectus/right) enantiomer or the S-(sinister/left) enantiomer. Of the atoms commonly present in molecules, nitrogen—the oldest known heteroatomic stereocentre—is the most difficult to stereochemically control.

As the most common organic cation, quaternary ammonium-based compounds are used daily within industrial, pharmaceutical, biological, and civilian contexts as surfactants, poragens, catalysts, agrochemicals, cosmetics and pharmaceuticals. The difficulty in enantioselective preparation of stereogenic nitrogen centres originates from their conformational instability. Carbon-based chiral centres are conformationally and configurationally locked. In contrast, the chirality of nitrogen atoms is often overlooked owing to this centre's generally rapid interconversion through inversion of nitrogen's lone pair enabled by quantum tunnelling (see J.-M. Lehn, Fortschr. Chem. Forsch. 15, 311-371 (1970)), which results in conformational instability of amines, eroding potential enantioenrichment at this centre.

Avoiding nitrogen inversion is possible in systems wherein the lone pair is essentially ‘locked’ in a stable conformation and configuration, thereby allowing for successful resolution of these centres. Simple alkylation of tertiary amines renders the nitrogen centre configurationally and conformationally locked, and when all substituents are different, chiral. Diastereoselective synthesis of nitrogen centres is successful under two regimes.

The first regime occurs when inversion of the lone pair at nitrogen is prevented for example, by locking the configuration of the lone pair within a bridgehead system, which makes it physically impossible to invert without destroying the ring system itself.

This configurationally stable system is most recognisable in the family of Cinchona alkoloids, isolated from the bark of the Cinchona genus (see U. —H. Dolling, P. Davis, and E. J. J. Grabowski, J. Am. Chem. Soc., 106, 446-447 (1984) and Cinchonidone, Cinchonine, Quinine and Quinidine structures shown in FIG. 1a). The N-bridgehead within the members of this family of alkaloids is a rare example of a configurationally stable nitrogen atom in naturally occurring molecules.

Examples of other molecules comprising locked nitrogen lone pairs within a bridgehead system, include (−)-sparteine, which is a naturally occurring chelating agent extracted from Lupinus mutabilis, Trögers base, and strychnine and brucine alkaloids extracted from the seeds of the Strychnos nux-vomica tree (see Scheme 1). Each of these molecules find use as organocatalysts, specifically for asymmetric transformations.

The second regime under which diastereoselective synthesis of nitrogen centres is successful occurs when the nitrogen stereocentre is fixed by transferring chirality from the carbon skeleton to the quaternary ammonium centre in a diastereoselective fashion (see D. R. Brown et al., J. Chem. Soc., 1184-1194 (1967)), as in pharmaceuticals such as methylnaltrexone (also known as Relistor™) ipratropium bromide (also known as Atrovent™, Apovent™ and Ipraxa™) and hyoscine butylbromide (also known as Buscopan™). The structures of the preferred enantiomers of these pharmaceuticals are shown in FIG. 1b. In each case, the preferred enantiomer has been found to have more effective pharmaceutical properties than the other enantiomer.

Accessing compounds where nitrogen is the sole stereogenic element is challenging. Since the first isolation in 1899 (see W. J. Pope and S. J. Peachey, J. Chem. Soc. 75, 1127-1131 (1899)), only a handful of kinetic resolution and spontaneous resolution based approaches have allowed the isolation of specific stereogenic nitrogen centres.

The use of stable enantiomeric quaternary ammonium salts to resolve racemic mixtures of organic molecules is known. The use of Cinchona alkaloids in the resolution of racemic biaryl diols such as 1,1′-bi-2-naphthol (BINOL) was reported by T. Toda and K. Tanaka in J. Org. Chem., 59, 5748-5751 (1994) and W. Yang, S. Jie and D. Kuiling in Tetrahedron, 56, 4447-4451 (2000). The reverse process has since been reported by E. Tayama and H. Tanaka in Tetrahedron Lett., 48, 4183-4185 (2007) and H.-F. Wu et al. in Helv. Chim. Acta, 92, 677-688 (2009), whereby kinetic resolution of quaternary ammonium centres bearing hydroxy group functional handles was achieved using BINOL. X-ray structural analysis confirmed the presence of a ternary complex comprising the quaternary ammonium cation, the halide counterion and BINOL. This complex is stabilised by hydrogen bonds between the hydroxy group functional handles and the halide counterion, and between the halide counterion and the hydroxy groups of BINOL (see FIG. 1c).

Tayama and Tanaka and H.-F. Wu et al. (supra) report methods for the kinetic resolution of racemic mixtures of quaternary ammonium centres bearing hydroxy group functional handles. These methods use BINOL to isolate specific enantiomers from the racemic mixture, giving a maximum possible yield of 50%. However, the generation of enantiomerically enriched quaternary ammonium salts from tertiary amines (such that an excess of one enantiomer is generated over the other) is not described in these documents.

There is a hitherto unmet need for a synthetic method to generate enantiomerically enriched tertiary or quaternary ammonium salts from tertiary amines that are chiral at the nitrogen centre. The present invention addresses this need.

### SUMMARY OF THE INVENTION

The inventors have found that reacting a tertiary amine that is chiral at the nitrogen centre with a compound of formula R—X, wherein R is different to any substituent on the nitrogen atom of the tertiary amine and X is a leaving group, under reversible conditions and in the presence of a non-racemic chiral compound having at least two substituents capable of coordinating to the tertiary or quaternary ammonium salt, is an effective method to produce an enantiomerically enriched tertiary or quaternary ammonium salt. The inventors have found that functional handles on the tertiary amine or on R—X, such as hydroxy groups, are not required for the method to work effectively, and that the method is surprisingly general, tolerating tertiary amines with a wide range of different functionalities and without functionality.

Without wishing to be bound by theory, it is understood that, unlike kinetic resolution methods used to isolate specific tertiary or quaternary ammonium salt enantiomers from racemic mixtures (such as those reported by Tayama and Tanaka and H.-F. Wu et al. (supra)), the method of the invention is driven by a thermodynamic adductive crystallisation process, which is responsible for the observed enantioselectivity. In contrast with kinetic resolution methods, the method of the invention promotes increased selectivity over time by a self-corrective process.

The tertiary or quaternary ammonium salt may be isolated from the reaction mixture as a ternary complex comprising the tertiary or quaternary ammonium salt and the chiral compound. The inventors have found that recrystallising the ammonium salt, when isolated in this way, significantly increases the degree of enantioenrichment.

Viewed from a first aspect, the invention provides a method of making an enantiomerically enriched tertiary or quaternary ammonium salt comprising reacting a tertiary amine with a compound of formula R—X to form a tertiary or quaternary ammonium salt, wherein the tertiary amine is chiral at the nitrogen atom, R is different to any substituent on the nitrogen atom of the tertiary amine and X is a leaving group and wherein the reacting is effected under reversible conditions in the presence of a non-racemic chiral compound having at least two substituents capable of coordinating to the tertiary or quaternary ammonium salt.

Viewed from a second aspect, the invention provides for the use of a non-racemic chiral compound in the synthesis of an enantiomerically enriched tertiary or quaternary ammonium salt from a tertiary amine, wherein the chiral compound has at least two substituents capable of coordinating to the tertiary or quaternary ammonium salt. The chiral compound may be as defined in the first aspect. The synthesis may be according to the method of the first aspect.

Further aspects and embodiments of the invention will be evident from the discussion that follows below.

### BRIEF DESCRIPTION OF THE FIGURES

FIG. 1: 1a—examples of molecules in which the lone pair at nitrogen is “locked”, within a bridgehead system, in a stable enantiomeric configuration; 1b—examples of molecules in which the nitrogen stereocentre is fixed by transferring chirality from the carbon skeleton to the quaternary ammonium centre in a diastereoselective fashion; 1c—kinetic resolution of quaternary ammonium salts comprising hydroxy group functional handles via molecular recognition with BINOL.

FIG. 2: enantioselective recognition of quaternary ammonium salts using BINOL enantiomers. a—a schematic showing the recognition of chiral quaternary ammonium salts with enantiopure BINOL. The enantiopurity of the quaternary ammonium salts may be assessed by NMR analysis, whereby counterion exchange is first carried out with a chiral shift reagent ((R,Λ)-BINPHAT) to form diastereomeric salts with characteristic NMR signals. NMR spectrum i) comprises signals corresponding to 1*.(R,Λ)-BINPHAT, NMR spectrum ii) comprises signals corresponding to 1.(R,Λ)-BINPHAT and NMR spectrum iii) comprises signals corresponding to (ent)-1*.(R,Λ)-BINPHAT. b—enantioselective recognition is exemplified with a range of quaternary ammonium salts, with X-ray crystal structures identifying the configuration of each quaternary ammonium centre. c—¹H NMR spectra showing a shift in the NMR signals corresponding to a quaternary ammonium salt on addition of BINOL, demonstrating solution phase recognition of the ammonium salt. d—unit cell and Hirshfeld plot of a ternary complex comprising (R)-BINOL complexed to the preferred quaternary ammonium salt enantiomer. e—unit cell and Hirshfeld plot of a ternary complex comprising (R)-BINOL complexed to the disfavoured quaternary ammonium salt enantiomer.

FIG. 3: dynamic behaviour of quaternary ammonium salts in solution. a—a schematic showing an equilibrium between a quaternary ammonium salt and corresponding tertiary amine and allyl bromide. (I)) ¹H NMR signals corresponding to the quaternary ammonium salt in dilute conditions at time t=0 and (II)) ¹H NMR signals corresponding to the tertiary amine and allyl bromide after heating this solution, indicating complete de-alkylation. (III)) ¹H NMR signals corresponding to the tertiary amine and allyl bromide in concentrated solution at time t=0, and (IV)) ¹H NMR signals corresponding to the tertiary amine, allyl bromide and the quaternary ammonium salt after heating this solution, indicating the formation of the quaternary ammonium salt. b—a schematic showing an equilibrium between a quaternary ammonium salt and corresponding tertiary amine and benzyl bromide. (I.) and (III.) racemisation of an enriched quaternary ammonium salt, monitored by optical rotation. (II.) and (IV.) ¹H NMR signals corresponding to the quaternary ammonium salt and tertiary amine, confirming the presence of the ammonium salt in the sample after complete loss of optical activity.

FIG. 4: enantioselective synthesis of ammonium cations. a—a schematic showing the enantioselective synthesis of quaternary ammonium cations from tertiary amines and compounds of formula R—X using BINOL. b—enantioselective synthesis is exemplified showing the formation and isolation of both enantiomeric forms of a range of quaternary ammonium salts. c—X-ray crystal structures of some ternary complexes. d—the isolated yield and enantioenrichment of a quaternary ammonium salt as the reaction progresses. e—a proposed model for predicting the enantioselectivity based on the order of the steric bulk of the groups attached to the quaternary ammonium centre. f—the mechanism of the enantioselective reaction. g—X-ray crystal structures of the two different enantiomers of a quaternary ammonium salt. h—supramolecular recognition of BINOL with pseudoenantiomeric and enantiomeric ammonium salts (conditions for processes (a) and (b) are stirring in acetonitrile at room temperature for 18 hours; and the conditions for process (c) are stirring in a mixture of ethyl acetate, dilute hydrochloric acid and deionised water for 1 hour)

FIG. 5: ¹H NMR signals showing solution state enantioselective recognition of (rac)-1b using a range of non-racemic chiral compounds.

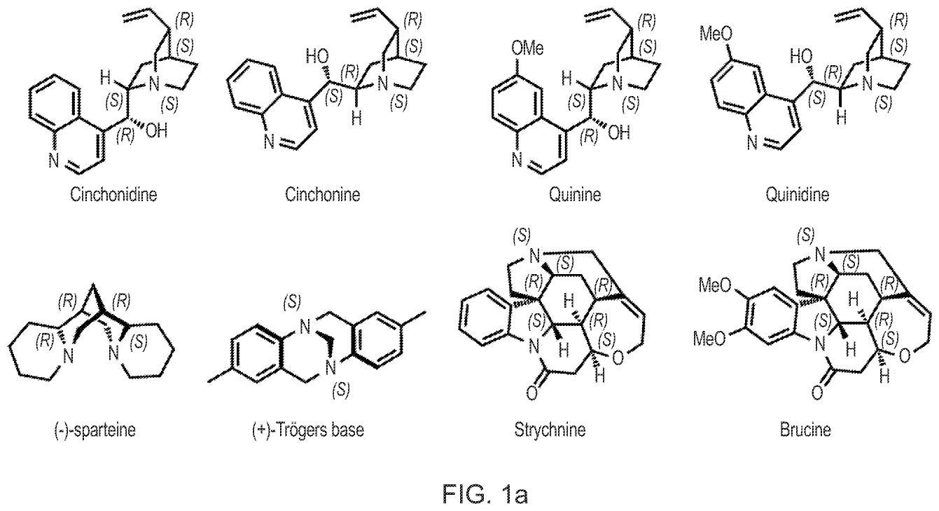

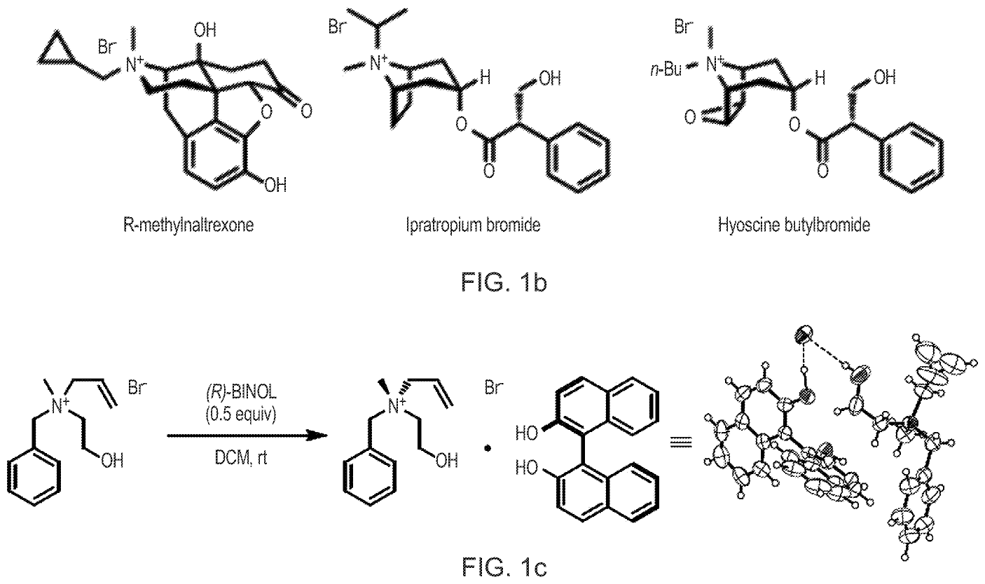

### DETAILED DESCRIPTION OF THE INVENTION

As described above, the inventors have found that reacting a tertiary amine that is chiral at the nitrogen centre with a compound of formula R—X, wherein R is different to any substituent on the nitrogen atom of the tertiary amine and X is a leaving group, under reversible conditions and in the presence of a non-racemic chiral compound having at least two substituents capable of coordinating to the tertiary or quaternary ammonium salt, is an effective method to produce an enantiomerically enriched tertiary or quaternary ammonium salt.

In the discussion that follows, reference is made to a number of terms, which are to be understood to have the meanings provided below, unless a context indicates to the contrary. The nomenclature used herein for defining compounds, in particular the compounds described herein, is intended to be in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 68, 2287-2311 (1996)). For the avoidance of doubt, if an IUPAC rule is contrary to a definition provided herein, the definition herein is to prevail.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof will be understood to imply the inclusion of a stated element, integer or step, or group of elements, integers or steps, and the exclusion of any other element, integer or step or group of elements, integers or steps.

The term “about” herein, when qualifying a number or value, is used to refer to values that lie within ±5% of the value specified. For example, if a temperature is defined as about 30° C. to about 70° C., temperatures of 28.5° C. to 73.5° C. are included.

The term “enantiomerically enriched” when used to describe a compound, refers to a chiral compound comprising more of one enantiomer than the other, e.g. more than 60% of one enantiomer. As described above, chirality is a property of molecules that do not possess an internal plane or point of symmetry and that may exist in one of two distinguishable, non-superposable mirror image forms—the R-(rectus/right) enantiomer or the S-(sinister/left) enantiomer.

R- and S-enantiomers are distinguishable by the direction of priority of the substituents attached to the chiral centre. Priority is based on the atomic number (proton number) of the first atom of the substituent. For example, if a quaternary ammonium cation is of formula [N(CH₃)(CH₂C₆H₅)(C₆H₅)(OMe)]⁺, the priority of the substituents (from lowest to highest) is in the order of CH₃<CH₂C₆H₅<C₆H₅<OMe. In the case of CH₃, CH₂C₆H₅ and C₆H₅, the first atom of the substituent is carbon. In order to distinguish priority of these two substituents, the second atoms of the substituents are taken into account. For CH₃, all of the second atoms are hydrogen whereas for CH₂C₆H₅, two of the second atoms are hydrogen and one is carbon, and for C₆H₅, the second atoms are all carbon. Since carbon has a higher atomic number than hydrogen, it takes priority. Hence, C₆H₅ has a greater priority than CH₂C₆H₅, which in turn has a greater priority than CH₃. To distinguish whether an enantiomeric tertiary or quaternary ammonium cation is R or S, the chiral nitrogen centre is oriented so that the lowest-priority of the four substituents (e.g. CH₃) is pointed away from the plane of view. If the priority of the remaining three substituents decreases in a clockwise direction, the enantiomer is R, and if the priority decreases in a counterclockwise direction, the enantiomer is S.

The term “tertiary ammonium salt” refers to derivatives of ammonium salts [NH₄]⁺[X]⁻ in which three of the hydrogen atoms bonded to nitrogen are replaced with hydrocarbyl groups, each of which optionally comprises one or more heteroatoms. The tertiary ammonium salts of the invention are chiral at the nitrogen centre. Consequently, each of the groups bound to the nitrogen centre are structurally different to one other.

The term “quaternary ammonium salt” refers to derivatives of ammonium salts in which all four of the hydrogen atoms bonded to nitrogen are replaced with hydrocarbyl groups, each of which optionally comprises one or more heteroatoms. As with the tertiary ammonium salts, the quaternary ammonium salts of the invention are chiral at the nitrogen centre. Consequently, each of the four hydrocarbyl groups bound to the nitrogen centre are structurally different to one other.

A tertiary amine is a derivative of ammonia (NH₃), in which all three hydrogen atoms are replaced with hydrocarbyl groups, each of which optionally comprises one or more heteroatoms. A tertiary amine that is chiral at the nitrogen centre is one in which the nitrogen centre is bound to three different hydrocarbyl groups. Such a tertiary amine may be under rapid conformational exchange between its R- and S-enantiomers. Without being bound by theory, the conformational exchange between the enantiomers of a chiral tertiary amine typically occurs via inversion of the tertiary amine at the nitrogen atom (pyramidal inversion). Conformational exchange may be hindered or terminated when the nitrogen atom of the tertiary amine is part of a monocycle or polycycle. Tertiary amines that are chiral at the nitrogen centre are capable of forming a chiral tertiary or quaternary ammonium cation in a single step-reaction of the lone pair of electrons on the nitrogen with a proton or hydrocarbyl that differs from the three hydrocarbyl groups already bound to the nitrogen centre.

Any heteroatoms present within the hydrocarbyl groups of the tertiary amine that may themselves, or as part of functionality to which they form part, react with R—X are typically protected with protecting groups. The skilled person is able to determine which protecting groups are appropriate for the protection of which functional groups. Ideally, therefore, the protected functional groups are stable under the conditions used for the method of the invention, and allow the chiral nitrogen centre of the tertiary amine to react with R—X. For the avoidance of doubt, tertiary amines that have been modified to protect any functional groups with a protecting group, are within the scope of this invention.

The term “hydrocarbyl” defines univalent groups derived from hydrocarbons by removal of a hydrogen atom from any carbon atom, wherein the term “hydrocarbon” refers to compounds consisting of hydrogen and carbon only. Where a hydrocarbyl is disclosed as optionally comprising one or more heteroatoms, any carbon or hydrogen atom on the hydrocarbyl may be substituted with a heteroatom or a functional group comprising a heteroatom, provided that valency is satisfied. One or more heteroatoms may be selected from the group consisting of oxygen, nitrogen, sulfur, fluorine, boron, bromine, chlorine, phosphorus and iodine.

Oxygen and sulfur heteroatoms or functional groups comprising these heteroatoms may replace —H or —CH₂— of a hydrocarbyl, provided that, when —H is replaced, oxygen or the functional group comprising oxygen binds to the carbon originally bound to the —H as either ═O (replacing two —H) or —OH (replacing one —H), and sulfur or the functional group comprising sulfur binds to the carbon atom originally bound to the —H as either ═S (replacing two —H) or —SH (replacing one —H). When methylene (—CH₂—) is replaced, oxygen binds to the carbon atoms originally bound to —CH₂— as —O— and sulfur binds to the carbon atoms originally bound to —CH₂— as —S—.

Nitrogen heteroatoms or functional groups comprising nitrogen heteroatoms may replace —H, —CH₂—, or —CH═, provided that, when —H is replaced, nitrogen or the functional group comprising nitrogen binds to the carbon originally bound to the —H as ≡N (replacing three —H), ═NH (replacing two —H) or —NH₂ (replacing one —H); when —CH₂— is replaced, nitrogen or the functional group comprising nitrogen binds to the carbon atoms originally bound to —CH₂— as —NH—; and when —CH═ is replaced, nitrogen binds to the carbon atoms originally bound to —CH═ as —N═.

Fluorine, bromine, chlorine and iodine heteroatoms may replace —H, wherein these heteroatoms bind to the carbon originally bound to the —H as —F, —Br, —Cl or —I, respectively.

Boron heteroatoms or functional groups comprising boron heteroatoms may replace —H, —CH₂—, or —CH═, provided that, when —H is replaced, boron or the functional group comprising boron binds to the carbon originally bound to the —H as —BR₂ (replacing one —H); when —CH₂— is replaced, boron or the functional group comprising boron binds to the carbon atoms originally bound to —CH₂— as —BR—; and when —CH═ is replaced, boron binds to the carbon atoms originally bound to —CH═ as —B═. R of —BR₂ or —BR— may be OH, OR′ or hydrocarbyl, where R′ is hydrocarbyl.

Phosphorus heteroatoms or functional groups comprising phosphorus heteroatoms may replace —H, —CH₂—, or —CH═, provided that, when —H is replaced, phosphorus or the functional group comprising phosphorus binds to the carbon originally bound to the —H as —PR₂ or —PR₃₂ (replacing one —H); when —CH₂— is replaced, phosphorus or the functional group comprising phosphorus binds to the carbon atoms originally bound to —CH₂— as —PR— or —PR₂—; and when —CH═ is replaced, phosphorus binds to the carbon atoms originally bound to —CH═ as —P═ or —PR═. R of —PR₂, —PR₃, —PR—, —PR₂—, —P═ or —PR═ may be OH, OR′, oxo (═O) or hydrocarbyl, where R′ is hydrocarbyl.

Where a hydrocarbyl optionally comprises one or more heteroatoms selected from the group consisting of oxygen, nitrogen, sulfur, fluorine, boron, bromine, chlorine, phosphorous and iodine, the hydrocarbyl typically optionally comprises one or more moieties selected from the group consisting of hydroxy, ether, keto (C═O), ester, amino, thiol, thioether (sulfide), fluoro, boronic acid (B(OH)₂), boronate ester (such as pinacol boronate (4,4,5,5-tetramethyl-1,3,2-dioxaborolane)), bromo, chloro, phosphine, phosphonate and iodo.

In some embodiments, where a hydrocarbyl optionally comprises one or more bromine, phosphorus and/or iodine atoms, the bromine, phosphorus and/or iodine atoms are bonded to an sp²-hybridised carbon atom. In other words, in some embodiments, the hydrocarbyl comprises one or more sp²-hybridised carbon atoms optionally substituted with bromine, phosphorus and/or iodine atoms. sp²-hybridised carbon atoms (of formula —CH═) may be part of optionally substituted C₂-C₆alkenyl, C₆-C₁₀aryl, CO₆-C₂₄biaryl, C₆-C₁₀arylC₁-C₆alkyl, C₆-C₂₄biarylC₁-C₆alkyl, C₆-C₁₀arylacyl, C₆-C₂₄biarylacyl, indolyl and tetrahydroquinolinyl, morphine, nalorphine, naltrexone, oxymorphone or atropine.

Where a hydrocarbyl optionally comprises one or more heteroatoms selected from the group consisting of oxygen, nitrogen, sulfur and fluorine, the hydrocarbyl typically optionally comprises one or more moieties selected from the group consisting of hydroxy, ether, keto (C═O), ester, amino, thiol, thioether (sulfide) and fluoro.

An ether is a group of formula ROR, an ester is a group of formula RC(O)OR and a thioether is a group of formula RSR, where each R is an independently an optionally substituted hydrocarbyl.

The term “racemic” when used to describe a compound may be used interchangeably with the term “racemate” and refers to a chiral compound comprising n equimolar mixture of a pair of enantiomers. Consequently, a racemate does not exhibit optical activity. The term “racemisation” refers to the production of a racemate from a chiral starting material in which one enantiomer is present in excess. The term “non-racemic” when used to describe a compound, refers to a chiral compound comprising more of one enantiomer than the other, e.g. more than 50% of one enantiomer. The term “non-racemic” may be used interchangeably with the term “enantiomerically enriched”.

The term “alkyl” is well known in the art and defines univalent groups derived from alkanes by removal of a hydrogen atom from any carbon atom, wherein the term “alkane” is intended to define acyclic branched or unbranched hydrocarbons having the general formula CₙH₂ₙ₊₂, wherein n is an integer 1. C₁-C₄alkyl refers to any one selected from the group consisting of methyl, ethyl, n-propyl, iso-propyl, n-butyl, sec-butyl, iso-butyl and tert-butyl.

The term “alkenyl” defines univalent groups derived from alkenes by removal of a hydrogen atom from any carbon atom, wherein the term “alkene” is intended to define acyclic branched or unbranched hydrocarbons having one carbon-carbon double bond and the general formula CₙH₂ₙ, where n is an integer ≥2. C₂-C₄alkenyl refers to any one selected from the group consisting of ethenyl, prop-1-enyl, prop-2-enyl, 1-methyl-ethenyl, but-1-enyl, but-2-enyl, but-3-enyl, 1-methyl-prop-1-enyl, 1-methyl-prop-2-enyl, 2-methyl-prop-1-enyl, and 2-methyl-prop-2-enyl.

The term “alkynyl” defines univalent groups derived from alkynes by removal of a hydrogen atom from any carbon atom, wherein the term “alkyne” is intended to define acyclic branched or unbranched hydrocarbons having one carbon-carbon triple bond and the general formula CₙH₂ₙ₋₂, where n is an integer ≥2. C₂-C₄alkynyl refers to any one selected from the group consisting of ethynyl, prop-1-ynyl, prop-2-ynyl, but-1-ynyl, but-2-ynyl, but-3-ynyl, and 1-methyl-prop-2-ynyl.

The term “aryl” is well known in the art and defines all univalent groups formed on removing a hydrogen atom from an arene ring carbon. The term “arene” defines monocyclic or polycyclic aromatic hydrocarbons, where “aromatic” defines a cyclically conjugated molecular entity with a stability (due to delocalisation) significantly greater than that of a hypothetical localised structure. The Hückel rule is often used in the art to assess aromatic character; monocyclic planar (or almost planar) systems of trigonally (or sometimes digonally) hybridised atoms that contain (4n+2) π-electrons (where n is a non-negative integer) will exhibit aromatic character. The rule is generally limited to n=0 to 5.

The term “heteroaryl” refers to compounds derived from aryls by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2).

The term “biaryl” refers to univalent groups formed formally by removal of a hydrogen atom from a biarene ring carbon, wherein the term “biarene” defines bicyclic aromatic hydrocarbons, such as biphenyl or binaphthyl.

The term “arylalkyl” such as “C₆-C₁₀arylC₁-C₆alkyl” refers to univalent groups formed formally by removal of a hydrogen atom from the alkane portion of an arylalkane, such as the removal of a hydrogen atom from the methyl group of toluene to form a benzyl group. Similarly, the term “biarylalkyl” such as “C₆-C₂₄biarylC₁-C₆alkyl” refers to univalent groups formed formally by removal of a hydrogen atom from the alkane part of a biarylalkane.

The term “arylacyl” such as “C₆-C₁₀arylacyl” refers to univalent groups formed formally by removal of a hydrogen atom from the ethanone portion (—C(O)CH₃) of an arylethanone, such as the removal of a hydrogen atom from the ethanone portion of phenylethanone (acetophenone) to form phenacyl. Similarly, the term “biarylacyl” such as “C₆-C₂₄biarylacyl” refers to univalent groups formed formally by removal of a hydrogen atom from the ethanone portion of a biarylethanone.

The term “cycloalkyl” defines all univalent groups derived from cycloalkanes by removal of a hydrogen atom from a ring carbon atom. The term “cycloalkane” defines saturated monocyclic and polycyclic branched or unbranched hydrocarbons, where monocyclic cycloalkanes have the general formula CₙH₂ₙ, wherein n is an integer ≥3.

The term “cycloalkylalkyl”, such as “C₃-C₈acycloalkylC₁-C₆alkyl”, defines univalent groups formed formally by removal of a hydrogen atom from the alkane portion of a cycloalkylalkane, such as the removal of a hydrogen atom from the methane substituent of cyclohexylmethane to form a cyclohexylmethyl group. A notable cycloalkylalkyl group is cyclopropylmethyl.

Where a group is described as being optionally substituted with a functional group such as any one or a combination of the group consisting of C₁-C₆alkyl, C₂-C₆alkenyl, C₂-C₆alkynyl, hydroxy, oxo and amino, one or more hydrogen atoms of the group may be replaced with C₁-C₆alkyl, C₂-C₆alkenyl, C₂-C₆alkynyl, hydroxy, oxo and/or amino, provided that valency is satisfied. For example, where the group is substituted with a C₁-C₆alkyl, C₂-C₆alkenyl, C₂-C₆alkynyl, hydroxy, oxo or amino, one hydrogen atom of the group is replaced with the C₁-C₆alkyl, C₂-C₆alkenyl, C₂-C₆alkynyl, hydroxy, oxo or amino. Where the group is substituted with an oxo, two hydrogen atoms of a —CH₂— on the group are replaced with the oxo, forming a carbonyl —C(O)—.

An amino may be a primary (—NH₂), secondary (—NRH) or tertiary (—NR₂) amino, where R is, or each R is independently, a hydrocarbyl group. Often, where the amino is a secondary or tertiary amino, it is a C₁-C₆alkylamino or diC₁-C₆alkylamino.

The Taft steric substituent constant (Es) is a measure of the steric bulk of a substituent and is calculated from the rate of acid-catalysed ester hydrolysis of an ester of formula R′O₂Me:

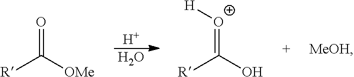

where R′ is the substituent of interest.

Es is calculated from

$${E}_{s} = log ( \frac{{k}_{s}}{{k}_{{CH}_{3}}} ) ,$$

where kₛ is the reaction rate of acid-catalysed ester hydrolysis of R′O₂Me and k_{CH}_{₃} is the reaction rate of acid-catalysed ester hydrolysis of MeO₂Me. The more steric the substituent of interest (R′), the less likely it is that a water molecule will bind to the carbonyl of the ester, and the slower the reaction rate. The Es value of methyl (used as the reference reaction) is set to 0.00. Consequently, more negative Es values indicate R′ groups with a greater steric bulk than methyl and more positive Es values indicate R′ groups with a smaller steric bulk than methyl. The Es values of some common substituents are: hydrogen (1.24), ethyl (−0.07), n-propyl (−0.36), iso-propyl (−0.47), n-butyl (−0.39), tert-butyl (−1.54) and phenyl (−2.58). For an overview of the Taft steric substituent constant see M. S. Sigman and J. J. Miller, J. Org. Chem., 2009, 74, 7633-7643; and R. W. Taft, J. Am. Chem. Soc., 1952, 74, 11, 2729-2732. The former describes the application of the Taft steric parameter to asymmetric catalysis and the latter provides a more comprehensive list of substituents and their Es values (see A values given under the sub-heading “Acid-catalyzed”, and under the heading “Substituent in acyl component” of Table 1 on page 2730).

The term “atropisomeric” refers to a molecule that may be isolated as one of two enantiomers that differ as a result of restricted rotation about a single bond. This is also known as axial chirality—restricted rotation about a single bond brings about a chiral axis. Atropisomerism is often exhibited by ortho-substituted biphenyls, wherein rotation about the bond connecting the two phenyl groups is restricted by steric hindrance between the ortho-substituents.

The term “protecting group” is used synonymously in the art with the term “protective group”, and is used in the temporary chemical transformation of a reactive group into a group that does not react under conditions where the non-protected group reacts. An ideal protecting group is one that reacts selectively to only protect the reactive group(s) that are not intended to react but that would otherwise react under the conditions used. Ideally, the resultant protected group is stable under these conditions. A desirable protecting group is selectively removed under conditions that do not detrimentally effect the regenerated functional group. For a comprehensive review of common protecting groups, see P. G. M. Wuts, “Greene's Protective Groups in Organic Synthesis”, 5^{th} Edition, John Wiley & Sons, Inc., Hoboken, New Jersey (2014).

The invention provides a method of making an enantiomerically enriched tertiary or quaternary ammonium salt comprising reacting a tertiary amine with a compound of formula R—X to form a tertiary or quaternary ammonium salt, wherein the tertiary amine is chiral at the nitrogen atom, R is different to any substituent on the nitrogen atom of the tertiary amine and X is a leaving group and wherein the reacting is effected under reversible conditions in the presence of a non-racemic chiral compound having at least two substituents capable of coordinating to the tertiary or quaternary ammonium salt.

In conceiving this invention, the inventors postulated that, in order to make an enantiomerically enriched tertiary or quaternary ammonium salt, it would be beneficial to meet three conditions: (i) a general recognition process allowing discrimination between the two enantiomeric forms of a tertiary or quaternary ammonium centre; (ii) a temporarily dynamic stereochemistry of the nitrogen centre of the tertiary or quaternary ammonium salt, allowing interconversion between the two enantiomeric forms; and (iii) compatibility of conditions (i) and (ii) and stabilisation of one enantiomer leading to a thermodynamically driven resolution.

Condition (i) may be met through the use of a non-racemic chiral compound having at least two substituents capable of coordinating to the tertiary or quaternary ammonium salt. It is believed that each enantiomer of the chiral compound coordinates more favourably with one of the two ammonium salt enantiomers, thereby discriminating one from the other.

Condition (ii) may be met by effecting the method of the invention under reversible conditions, by which is meant that reaction of R—X is reversible such that the tertiary or quaternary ammonium salt and the tertiary amine are in equilibrium with one another. The position of equilibrium may be altered by changing the reaction conditions under which the method is effected. Protic solvents of high polarity, i.e. those with a dielectric constant at 25° C. of ≥17, such as n-butyl alcohol, iso-propyl alcohol, n-propyl alcohol, ethanol, methanol and water, are likely to shift equilibrium to the far right, resulting in highly stable tertiary or quaternary ammonium salts. However, the hydrogen bonding capability of the solvent stabilizes the halide counterion, retarding dynamic behaviour and hinders the reverse reaction. In contrast, aprotic polar solvents such as acetonitrile, shift the equilibrium to the far right in a similar fashion whilst still allowing the reverse reaction to occur, allowing interconversion of the two enantiomeric forms. Likewise, aprotic solvents of low-polarity, i.e. those with a dielectric constant at 25° C. of ≤10, such as dichlorobenzene, dichloromethane, tetrahydrofuran, chlorobenzene and chloroform, are likely to shift equilibrium to the middle-left, in favour of the tertiary amine whilst allowing the halide counterion to perform the reverse reaction and racemise the ammonium cation. Importantly, all such modulations should not promote reaction of the non-racemic chiral compound with R—X, which reaction could render this species unable to complex to the chiral ammonium cation.

The position of equilibrium may be altered in favour of the tertiary or quaternary ammonium salt or tertiary amine by applying Le Chatelier's principle: if a constraint (such as a change in pressure, temperature, or concentration of a reactant) is applied to a system in equilibrium, the equilibrium will shift so as to counteract the effect of the constraint. Formation of the tertiary or quaternary ammonium salt is favoured, i.e. equilibrium is shifted to the right, by increasing the concentration of the tertiary amine and/or R—X, increasing the pressure under which the reaction is conducted, and/or decreasing the temperature. Formation of the tertiary amine is favoured by applying the opposite constraints. Often, a fast interconversion between tertiary amine and tertiary or quaternary ammonium salt is preferred, with equilibrium favouring the formation of the tertiary or quaternary ammonium salt, i.e. positioned to the right. An increased concentration of R—X also leads to a more rapid interconversion between the ammonium salt enantiomers.

Condition (iii) requires compatibility of conditions (i) and (ii) and stabilisation of one enantiomer leading to a thermodynamically driven resolution. The combination of condition (i) and condition (ii) places several restrictions that are non-obvious to one skilled in the art. Condition (iii) may be met by the method of the invention through reacting the tertiary amine and R—X under reversible conditions in the presence of the non-racemic chiral compound having at least two substituents capable of coordinating to the tertiary or quaternary ammonium salt, which coordination is also reversible. Reaction with R—X may form either enantiomer of the tertiary or quaternary ammonium salt. However, each enantiomer of the non-racemic chiral compound preferentially coordinates to one ammonium salt enantiomer over the other. The ternary complex comprising the tertiary or quaternary ammonium salt and the chiral compound is thermodynamically more stable when the chiral compound coordinates to the preferred enantiomer. The less stable ternary complex, comprising the chiral compound coordinated to the least preferred tertiary or quaternary ammonium salt enantiomer, is more likely to dissociate to reform the least preferred tertiary or quaternary ammonium salt enantiomer. In general, dissociation requires elevated temperatures with respect to coordination. The least preferred enantiomer may then dissociate into the tertiary amine and R—X, and react again with R—X. Since the chiral compound is non-racemic, there is a greater amount of one enantiomer. This means that, over time, the more stable ternary complex comprising the enantiomer of the chiral compound that is in excess and the preferred tertiary or quaternary ammonium salt enantiomer accumulates. Thus, the enantioselectivity of the reaction increases over time by a self-corrective process.

The preferred tertiary or quaternary ammonium salt enantiomer is not necessarily that with the same chirality as the chiral compound, i.e. R-chiral compound does not necessarily complex preferentially with an R-tertiary or quaternary ammonium salt and S-chiral compound does not necessarily complex preferentially with an S-tertiary or quaternary ammonium salt.

As described above, a fast interconversion between tertiary amine and tertiary or quaternary ammonium salt is often preferred, with equilibrium favouring the formation of the tertiary or quaternary ammonium salt. Sometimes, aprotic solvents of low-polarity are used to promote interconversion between the tertiary amine and the ammonium salt. Sometimes, the method of the invention is carried out in any one or a combination of chloroform, dichloromethane, acetonitrile, acetone, dichlorobenzene, tetrahydrofuran and chlorobenzene. Often, the method of the invention is carried out in one solvent, which is typically chloroform, dichloromethane, acetonitrile or acetone. Most often, the method of the invention is carried out in chloroform.

As disclosed in the Examples section below, the method of the invention tolerates water and oxygen, thus solvents used in the method of the invention need not be dried, and the reacting may be effected open to the atmosphere.

Sometimes, interconversion between the tertiary amine and the tertiary or quaternary ammonium salt is promoted by effecting the reacting of the method of the invention at elevated temperatures, i.e. by heating. The reacting is often effected at temperatures of about 30° C. to about 70° C., about 35° C. to about 65° C., about 40° C. to about 60° C., or about 45° C. to about 55° C. Typically, the reacting is effected at temperatures of about 50° C. As described above, formation of the tertiary or quaternary ammonium salt is favoured (i.e. equilibrium is shifted to the right) by decreasing the temperature. However, low temperatures decrease the rate of conversion between the tertiary amine and the tertiary or quaternary ammonium salt. Typically, the benefit of a faster interconversion between the tertiary amine and the tertiary or quaternary ammonium salt at higher temperatures outweighs the disadvantageous shifting of equilibrium to favour formation of the tertiary amine.

Sometimes, interconversion between the tertiary amine and the tertiary or quaternary ammonium salt is promoted by using a lower concentration of tertiary amine. Often, the concentration of tertiary amine is about 0.05 M to about 2 M, about 0.1 M to about 1.5 M, about 0.2 M to about 1.2 M, about 0.3 M to about 1 M, about 0.4 M to about 0.8 M, or about 0.6 M. Typically, the concentration of the tertiary amine is about 0.6 M.

The amount of R—X used in the method of the invention may be selected to shift equilibrium to favour the formation of the tertiary or quaternary ammonium salt. In some embodiments, the ratio of tertiary amine to R—X is any one selected from the group consisting of 1:≥1, 1:≥1.2, 1:≥1.4, 1:≥1.6, 1:≥1.8 and 1:≥2. It is to be understood that reference herein to a ratio is to a molar ratio. Typically, the ratio of tertiary amine to R—X is 1:≥2.

The methods reported for the kinetic resolution of racemic mixtures of quaternary ammonium centres (Tayama and Tanaka and H.-F. Wu et al. (supra)) use a ratio of quaternary ammonium salt to BINOL of 1:0.5. This is because one specific enantiomer is isolated from a racemic mixture, giving a maximum possible yield of 50%. In contrast to these techniques, the method of the invention makes, or generates, an enantiomerically enriched tertiary or quaternary ammonium salt by reacting a tertiary amine with a compound of formula R—X, wherein the maximum possible yield of enantiomerically enriched tertiary or quaternary ammonium salt is 100%. Consequently, in some embodiments, the ratio of tertiary amine to non-racemic chiral compound used in the method of the invention is any one selected from the group consisting of 1:>0.5, 1:≥0.6, 1:≥0.7, 1:≥0.8, 1:≥0.9 and 1:≥1. In many embodiments, the ratio of tertiary amine to non-racemic chiral compound is 1:≥1.

Often, the non-racemic chiral compound comprises more than 60%, 70%, 80%, 90% or 95% of one enantiomer. Typically, the non-racemic chiral compound comprises more than 95% of one enantiomer. Often, the non-racemic chiral compound is enantiomerically pure, by which is meant that it comprises 99% of one enantiomer. As the enantiomeric purity of the non-racemic chiral compound increases, so too does the achievable extent of enantiomeric enrichment of the tertiary or quaternary ammonium salt.

The tertiary amine used in the method of the invention is chiral at the nitrogen centre. Accordingly, the hydrocarbyl groups of the tertiary amine, each of which is optionally substituted with one or more heteroatoms, are all different. In the method of the invention, the tertiary amine is reacted with a compound of formula R—X to form the tertiary or quaternary ammonium salt. The lone pair at the chiral nitrogen centre of the tertiary amine forms a bond with the R of R—X, the bond between R—X breaks leaving X⁻, which typically acts as a counterion that stabilises the resultant tertiary or quaternary ammonium cation.

As described above, the inventors have found that functional handles on the tertiary amine, such as hydroxy group functional handles are not required for the method to work effectively. This makes the method surprisingly general, tolerating tertiary amines with a wide range of different functionalities and no functionality. In some embodiments, the tertiary amine is of formula N(R¹)₃, wherein each R¹ is a different hydrocarbyl group optionally comprising one or more heteroatoms selected from the group consisting of oxygen, nitrogen, sulfur, fluorine, boron, bromine, chlorine, phosphorous and iodine. Alternatively, two of R¹ may together with the nitrogen atom to which they are attached form a cyclic or bicyclic N-containing hydrocarbon optionally comprising one or more heteroatoms selected from the group consisting of oxygen, nitrogen, sulfur, fluorine, boron, bromine, chlorine, phosphorous and iodine and the other R¹ may be a hydrocarbyl group optionally comprising one or more heteroatoms selected from the group consisting of oxygen, nitrogen, sulfur, fluorine, boron, bromine, chlorine, phosphorous and iodine. Since the tertiary amine of the method of the invention is chiral, where two of R¹ together with the nitrogen atom to which they are attached form a cyclic or bicyclic N-containing hydrocarbon, the resultant cyclic or bicyclic N-containing hydrocarbon is asymmetric. In another alternative, three of R¹ may together with the nitrogen atom to which they are attached form a bicyclic N-containing hydrocarbon optionally comprising one or more heteroatoms selected from the group consisting of oxygen, nitrogen, sulfur, fluorine, boron, bromine, chlorine, phosphorous and iodine. For the avoidance of doubt, the resultant N(R¹)₃ is chiral.

Often, when R¹ comprises one or more heteroatoms, it is, or they are independently, selected from the group consisting of oxygen, nitrogen, sulfur and fluorine, such as oxygen and nitrogen.

In some embodiments, the tertiary amine is of formula N(R¹)₃, and each R¹ is independently selected from the group consisting of C₁-C₆alkyl, C₂-C₆alkenyl, C₂-C₆alkynyl, C₆-C₁₀aryl, C₆-C₂₄biaryl, C₆-C₁₀arylC₁-C₆alkyl, C₆-C₂₄biarylC₁-C₆alkyl, C₆-C₁₀arylacyl, C₆-C₂₄biarylacyl, C₃-C₈cycloalkyl, C₃-C₈cycloalkylC₁-C₆alkyl and C₃-C₅heteroaryl, each of which may be optionally substituted with any one or a combination selected from the group consisting of hydroxy, oxo and amino.

In some embodiments, each R¹ is independently selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted with any one or a combination selected from the group consisting of hydroxy, oxo and amino. According to particular embodiments, the substituents are selected from hydroxy and/or amino. According to alternative embodiments, each R¹ group is unsubstituted.

In other embodiments, two R¹ groups together with the nitrogen atom to which they are attached form indolyl, tetrahydroquinolinyl, 3-azabicyclo[3.2.1]octanyl or camphidinyl, optionally substituted with any one or a combination selected from the group consisting of C₁-C₆alkyl, C₂-C₆alkenyl, C₂-C₆alkynyl, hydroxy, oxo and amino; and the other R¹ group is selected from the group consisting of C₁-C₆alkyl, C₂-C₆alkenyl, C₂-C₆alkynyl, C₆-C₁₀aryl, C₆-C₂₄biaryl, C₆-C₁₀arylC₁-C₆alkyl, C₆-C₂₄biarylC₁-C₆alkyl, C₆-C₁₀arylacyl, C₆-C₂₄biarylacyl, C₃-C₈cycloalkyl and C₃-C₈cycloalkylC₁-C₆alkyl, optionally substituted one or more times with any one or a combination selected from the group consisting of hydroxy, oxo and amino.

In still other embodiments, two R¹ groups together with the nitrogen atom to which they are attached form morpholino, pyrrolidino or piperidinyl, substituted with any one or a combination selected from the group consisting of C₁-C₆alkyl, C₂-C₆alkenyl, C₂-C₆alkynyl, hydroxy, oxo and amino such that the resultant N(R¹)₂ is asymmetric; and the other R¹ group is selected from the group consisting of C₁-C₆alkyl, C₂-C₆alkenyl, C₂-C₆alkynyl, C₆-C₁₀aryl, C₆-C₂₄biaryl, C₆-C₁₀acrylC₁-C₆alkyl, C₆-C₂₄biarylC₁-C₆alkyl, C₆-C₁₀arylacyl, C₆-C₂₄biarylacyl, C₃-C₈cycloalkyl and C₃-C₈cycloalkylC₁-C₆alkyl, optionally substituted one or more times with any one or a combination selected from the group consisting of hydroxy, oxo and amino.

According to particular embodiments, the substituents of N(R¹)₂ are any one or a combination selected from the group consisting of C₁-C₆alkyl, C₂-C₆alkenyl, C₂-C₆alkynyl, hydroxy, oxo and amino; and the other R¹ is selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted one or more times with any one or a combination selected from the group consisting of hydroxy, oxo and amino.

In further embodiments, the substituents of N(R¹)₂ are any one or a combination selected from the group consisting of C₁-C₆alkyl, C₂-C₆alkenyl, C₂-C₆alkynyl, hydroxy and amino; and the other R¹ group is selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted with one or more hydroxy and/or amino.

According to particular embodiments, the other R¹ group is unsubstituted.

In further embodiments, all three R¹ groups together with the nitrogen atom to which they are attached form 1,4-diazabicyclo[2.2.2]octane or 1-azabicyclo[2.2.2]octane substituted with any one or a combination selected from the group consisting of C₁-C₆alkyl, C₂-C₆alkenyl, C₂-C₆alkynyl, hydroxy, oxo and amino such that the resultant N(R¹)₃ is chiral.

In alternative embodiments, N(R¹)₃ is morphine, nalorphine, naltrexone, oxymorphone, or atropine:

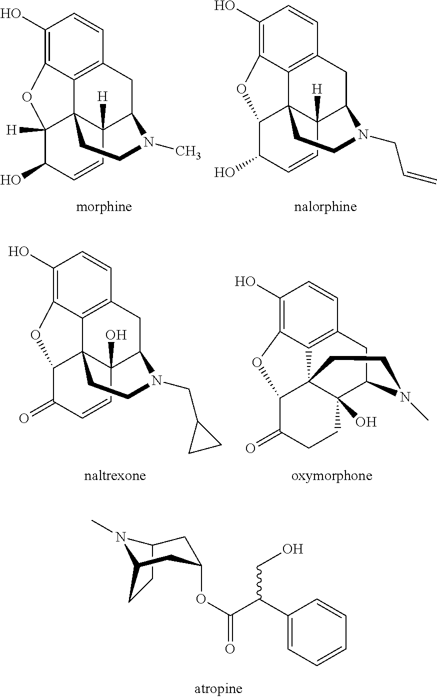

In some embodiments, at least one of R¹ is an optionally substituted phenyl group.

In each embodiment where one or more R¹, N(R¹)₂ or N(R¹)₃ is optionally substituted, when substituted, it is often protected with one or more protecting groups.

In some embodiments, amino is diC₁-C₆alkylamino. Often, amino is any one selected from the group consisting of dimethylamino, diethylamino, dipropylamino, di-iso-propylamino, dibutylamino, di-sec-butylamino, di-iso-butylamino and di-tert-butylamino. Typically, amino is any one selected from the group consisting of dimethylamino, diethylamino, di-iso-propylamino and di-tert-butylamino, most typically dimethylamino.

R of R—X is a hydrocarbyl group, which is different to each R¹, or a proton. In some embodiments, R is a hydrocarbyl which is different to each R¹. In these specific embodiments, the invention provides a method of making an enantiomerically enriched quaternary ammonium salt, i.e. the tertiary or quaternary ammonium salt is a quaternary ammonium salt. Sometimes, R is selected from the group consisting of C₁-C₆alkyl, C₂-C₆alkenyl, C₂-C₆alkynyl, C₆-C₁₀aryl, C₆-C₂₄biaryl, C₆-C₁₀arylC₁-C₆alkyl, C₆-C₂₄biarylC₁-C₆alkyl, C₃-C₃cycloalkyl and H. Often, R is selected from the group consisting of C₁-C₆alkyl, C₂-C₆alkenyl, C₂-C₆alkynyl, C₆-C₁₀aryl, C₆-C₁₀arylC₁-C₆alkyl, C₃-C₈cycloalkyl and H. Typically, R is C₂-C₆alkenyl or C₆-C₁₀arylC₁-C₆alkyl.

Sometimes, C₁-C₆alkyl is C₁-C₄alkyl; C₂-C₆alkenyl is C₂-C₄alkenyl; C₂-C₆alkynyl is C₂-C₄alkynyl; C₆-C₁₀aryl is phenyl; C₆-C₁₀arylacyl is phenylacyl; C₆-C₁₀arylC₁-C₆alkyl is phenylC₁-C₆alkyl, typically phenylC₁-C₄alkyl; C₃-C₈cycloalkyl is cyclohexyl; and/or C₃-C₈cycloalkylC₁-C₆alkyl is cyclohexylC₁-C₄alkyl. Often, phenylC₁-C₆alkyl is benzyl.

The inventors have found that enantioenrichment of the tertiary or quaternary ammonium salts that result from the method of the invention is improved when the difference in steric bulk between the substituents bound to the chiral nitrogen centre of the tertiary amine is larger. It was found that increasing the difference in the Taft steric substituent constant between the substituents most similar in size from 0.07 (difference between Eₛ of methyl and ethyl) to 0.47 (difference between Eₛ of methyl and iso-propyl) significantly increased enantioenrichment of the ammonium salts resultant from the method of the invention. In some embodiments, the difference in μs of each substituent on the tertiary amine is >0.07. In some embodiments, the tertiary amine has three substituents each of which is unconnected to the other two substituents and each has a different Taft steric substituent constant (Eₛ) and the Taft steric substituent constants differ by >0.07. Sometimes, the difference in Eₛ of each substituent on the tertiary amine is ≥0.47.

X of R—X is a leaving group, i.e. an atom or group that detaches from R subsequent to, during, or before the formation of a bond between the tertiary amine and R. On detachment, X becomes X⁻ and typically acts as counterion to the tertiary or quaternary ammonium salt resultant from the method of the invention. The skilled person is aware of suitable leaving groups for the method of the invention. Sometimes, X is selected from the group consisting of halo, triflate, tosylate, phosphate and acetoxy. Often, halo is bromo, iodo or chloro, such as bromo or iodo. Typically, X is bromo or iodo, most typically bromo.

In some embodiments, where X is chloro, triflate, tosylate, phosphate or acetoxy, the reacting is effected in the presence of iodide. Any source of iodide may be used. In some embodiments, the reacting is effected in the presence of tetrabutylammonium iodide. Without being bound by theory, reacting in the presence of iodide is understood by the inventors to promote the Finkelstein reaction, in which the iodide displaces X (see Li J. J. (2003) Finkelstein reaction. In: Name Reactions.

Springer, Berlin, Heidelberg). Since iodo is a superior leaving group to chloro, triflate, tosylate, phosphate or acetoxy, replacement of X with iodide promotes the reacting of the tertiary amine with R—X.

The reacting of the method of the invention is effected in the presence of a non-racemic chiral compound having at least two substituents capable of coordinating to the tertiary or quaternary ammonium salt. It is understood that the at least two substituents typically coordinate to the counterion of the tertiary or quaternary ammonium salt, which in turn coordinates to the tertiary or quaternary ammonium cation. Where X⁻ acts as counterion, the at least two substituents typically coordinate to X⁻. However, X® need not act as a counterion to the tertiary or quaternary ammonium cation. Counterions that are not derived from R—X may be added to the reaction mixture in the form of salts, such as potassium (K⁺X⁻) or sodium (Na⁺X⁻) salts.

In some embodiments, the at least two substituents capable of coordinating to the tertiary or quaternary ammonium salt are both —OH. The inventors have found that hydroxy substituents are able to coordinate to the ammonium salt via hydrogen bonding. Typically, the hydroxy substituents form hydrogen bonds with the counterion of the tertiary or quaternary ammonium cation, which in turn forms hydrogen bonds with the tertiary or quaternary ammonium cation itself.

In some embodiments, the chiral compound has two substituents capable of coordinating to the tertiary or quaternary ammonium salt. Often, each of the two substituents is independently selected from the group consisting of —OH and —NH₂. Typically, the two substituents are each —OH.

In some embodiments, the chiral compound is any one of structures (I), (Ib), (II), (III) or (IV):

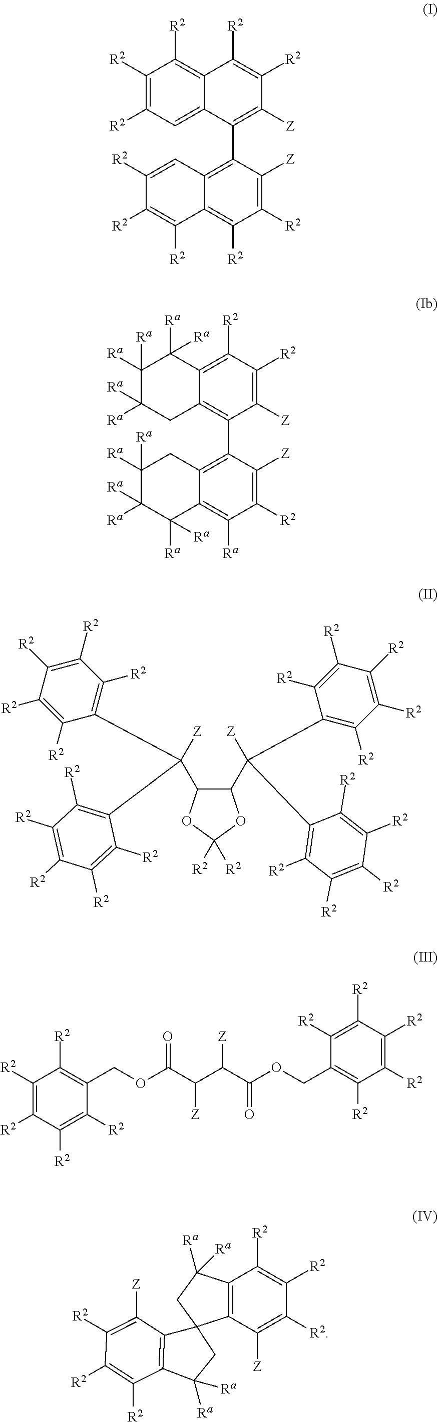

In some embodiments, the chiral compound is any one of structures (I) to (III),

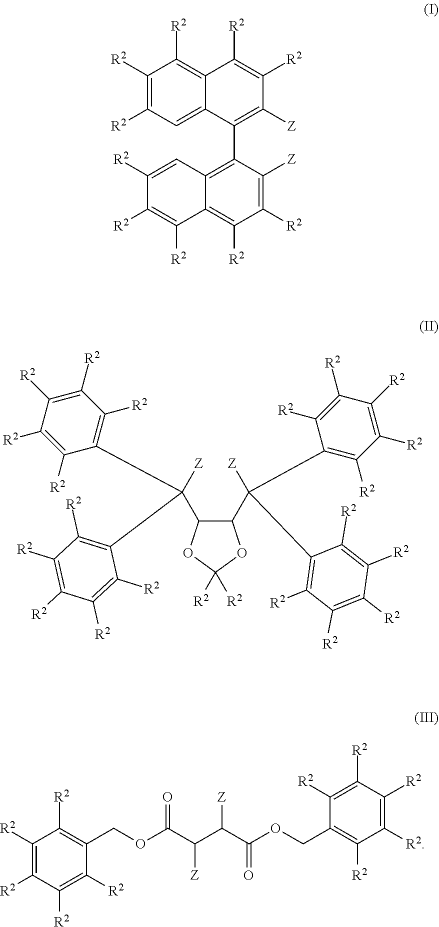

Each Z is independently selected from the group consisting of —OH and —NH₂. In some embodiments, each Z is —OH.

Each R² is independently selected from the group consisting of —H, halo, C₁-C₆alkyl, C₁-C₆alkoxy, C₂-C₆alkenyl, C₂-C₆alkynyl, C₆-C₁₀aryl, C₆-C₁₀arylC₁-C₆alkyl, mono-, di- or tri-C₁-C₆alkylC₆-C₁₀aryl, mono-, di- or tri-C₁-C₆alkoxyC₆-C₁₀aryl, mono-, di- or tri-C₁-C₆fluoroalkylC₆-C₁₀aryl, tri-C₆-C₁₀arylsilyl, C₃-C₈cycloalkyl, C₃-C₈cycloalkylC₁-C₆alkyl, mono-, di- or tri-C₁-C₆alkylC₃-C₈cycloalkyl, mono-, di- or tri-C₁-C₆fluoroalkylC₃-C₈cycloalkyl. Often, each R² is independently selected from the group consisting of —H, C₁-C₆alkyl, C₁-C₆alkoxy, C₆-C₁₀aryl, tri-C₁-C₆alkylC₆-C₁₀aryl, mono-C₁-C₆alkoxyC₆-C₁₀aryl, di-C₁-C₆alkoxyC₆-C₁₀aryl, di-C₁-C₆fluoroalkylC₆-C₁₀aryl, tri-C₆-C₁₀arylsilyl and C₃-C₈cycloalkyl.

In some embodiments, R² is independently selected from the group consisting of —H, C₁-C₆alkyl, C₁-C₆alkoxy, C₂-C₆alkenyl, C₂-C₆alkynyl, C₆-C₁₀aryl, C₆-C₁₀arylC₁-C₆alkyl, mono-, di- or tri-C₁-C₆alkylC₆-C₁₀aryl, mono-, di- or tri-C₁-C₆alkoxyC₆-C₁₀aryl, mono-, di- or tri-C₁-C₆fluoroalkylC₆-C₁₀aryl, tri-C₆-C₁₀arylsilyl, C₃-C₈cycloalkyl, C₃-C₆cycloalkylC₁-C₆alkyl, mono-, di- or tri-C₁-C₆alkylC₃-C₈cycloalkyl, mono-, di- or tri-C₁-C₆fluoroalkylC₃-C₈cycloalkyl. Often, each R² is independently selected from the group consisting of —H, C₁-C₆alkyl, C₁-C₆alkoxy, C₆-C₁₀aryl, tri-C₁-C₆alkylC₆-C₁₀aryl, mono-C₁-C₆alkoxyC₆-C₁₀aryl, di-C₁-C₆alkoxyC₆-C₁₀aryl, di-C₁-C₆fluoroalkylC₆-C₁₀aryl, tri-C₆-C₁₀arylsilyl and C₃-C₈cycloalkyl.

Each R^{a} is independently selected from the group consisting of —H, C₁-C₆alkyl, C₁-C₆alkoxy, C₂-C₆alkenyl, C₂-C₆alkynyl, C₆-C₁₀aryl, C₆-C₁₀arylC₁-C₆alkyl, mono-, di- or tri-C₁-C₆alkylC₆-C₁₀aryl, mono-, di- or tri-C₁-C₆alkoxyC₆-C₁₀aryl, mono-, di- or tri-C₁-C₆fluoroalkylC₆-C₁₀aryl, tri-C₆-C₁₀arylsilyl, C₃-C₈cycloalkyl, C₃-C₈cycloalkylC₁-C₆alkyl, -mono-, di- or tri-C₁-C₆alkylC₃-C₈cycloalkyl, mono-, di- or tri-C₁-C₆fluoroalkylC₃-C₈cycloalkyl. Often, each R² is independently selected from the group consisting of —H, C₁-C₆alkyl, C₁-C₆alkoxy, C₆-C₁₀aryl, tri-C₁-C₆alkylC₆-C₁₀aryl, mono-C₁-C₆alkoxyC₆-C₁₀aryl, di-C₁-C₆alkoxyC₆-C₁₀aryl, di-C₁-C₆fluoroalkylC₆-C₁₀aryl, tri-C₆-C₁₀arylsilyl and C₃-C₈cycloalkyl.

Sometimes, with respect to R² and R^{a} (such as R²), C₁-C₆alkyl is C₁-C₄alkyl; C₁-C₆alkoxy is C₁-C₄alkoxy, C₂-C₆alkenyl is C₂-C₄alkenyl; C₂-C₆alkynyl is C₂-C₄alkynyl; C₆-C₁₀aryl is phenyl; C₆-C₁₀arylC₁-C₆alkyl is phenylC₁-C₆alkyl, typically phenylC₁-C₄alkyl; -mono-, di- or tri-C₁-C₆alkylC₆-C₁₀aryl is mono-, di- or tri-C₁-C₄alkylphenyl; mono-, di- or tri-C₁-C₆alkoxyC₆-C₁₀aryl is mono-, di- or tri-C₁-C₆alkoxyphenyl; mono-, di- or tri-C₁-C₆fluoroalkylC₆-C₁₀aryl is mono-, di- or tri-C₁-C₆fluoroalkylphenyl; tri-C₆-C₁₀arylsilyl is triphenylsilyl; C₃-C₈cycloalkyl is cyclohexyl; C₃-C₈cycloalkylC₁-C₆alkyl is cyclohexylC₁-C₆alkyl, typically cyclohexylC₁-C₄alkyl; mono-, di- or tri-C₁-C₆alkylC₃-C₈cycloalkyl is mono-, di- or tri-C₁-C₆alkylcyclohexyl, typically mono-, di- or tri-C₁-C₄alkylcyclohexyl; and/or mono-, di- or tri-C₁-C₆fluoroalkylC₃-C₈cycloalkyl is mono-, di- or tri-C₁-C₆fluoroalkylcyclohexyl, typically mono-, di- or tri-C₁-C₄fluoroalkylcyclohexyl. Often, phenylC₁-C₆alkyl is benzyl; mono-, di- or tri-C₁-C₆fluoroalkylC₆-C₁₀aryl is mono-, di- or tri-trifluoromethylphenyl; and/or mono-, di- or tri-C₁-C₆fluoroalkylC₃-C₈cycloalkyl is mono-, di- or tri-trifluoromethylcyclohexyl. Sometimes, with respect to R², halo is bromo.

In some embodiments, each R^{a} is —H.

In some embodiments, each R² is independently selected from the group consisting of —H, halo, tri-isopropylphenyl, di-trifluoromethylphenyl and triphenylsilyl.

Sometimes, each R² is —H or halo.

In some embodiments, each R² is independently selected from the group consisting of —H, tri-isopropylphenyl, di-trifluoromethylphenyl and triphenylsilyl.

Typically, each R² is —H.

In some embodiments, the chiral compound of structure (I) is of structure (Ia):

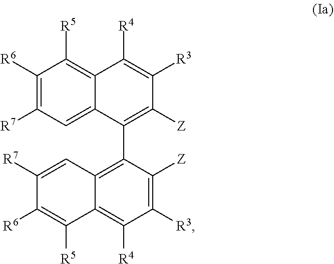

wherein Z is as defined for Z, above, and R³, R⁴, R⁵, R⁶ and R⁷ are as defined for R², above. For the avoidance of doubt, the two substituents within a pair of R³, R⁴, R⁵, R⁶ and R⁷ are identical. In other words, the compound of structure (Ia) has C₂-symmetry. In some embodiments, R⁶ and R⁷ are independently selected from the group consisting of —H, halo, C₁-C₆alkyl, C₁-C₆alkoxy, C₂-C₆alkenyl and C₂-C₆alkynyl, such as —H, halo, C₁-C₆alkyl and C₁-C₆alkoxy. In some embodiments, R⁶ and R⁷ are independently selected from the group consisting of —H, C₁-C₆alkyl, C₁-C₆alkoxy, C₂-C₆alkenyl and C₂-C₆alkynyl, such as —H, C₁-C₆alkyl and C₁-C₆alkoxy. In some embodiments, R⁶ or R⁷ is hydrogen. In some embodiments, R⁶ is halo. In some embodiments, R³, R⁴ and R⁵ are independently selected from the group consisting of C₆-C₁₀aryl, C₆-C₁₀arylC₁-C₆alkyl, mono-, di- or tri-C₁-C₆alkylC₆-C₁₀aryl, mono-, di- or tri-C₁-C₆alkoxyC₆-C₁₀aryl, mono-, di- or tri-C₁-C₆fluoroalkylC₆-C₁₀aryl, tri-C₆-C₁₀arylsilyl, C₃-C₈cycloalkyl, C₃-C₈cycloalkylC₁-C₆alkyl, mono-, di- or tri-C₁-C₆alkylC₃-C₈cycloalkyl, - and mono-, di- or tri-C₁-C₆fluoroalkylC₃-C₈cycloalkyl, such as C₆-C₁₀aryl, C₆-C₁₀arylC₁-C₆alkyl, mono-, di- or tri-C₁-C₆alkylC₆-C₁₀aryl, mono-, di- or tri-C₁-C₆alkoxyC₆-C₁₀aryl, mono-, di- or tri-C₁-C₆fluoroalkylC₆-C₁₀aryl and tri-C₆-C₁₀arylsilyl. In some embodiments, R⁵ is methoxy.

Synthetic routes to compounds of formula (I) and (Ia) are exemplified by J. J. Patel et al. in Angew. Chem. Int. Ed., 2018, 57, 9425-9429. Kinetic resolution of the resultant compounds may be achieved through complexation of the compounds with a chiral counterion, as described by B. A Jones et al. in Angew. Int. Ed., 2019, 58, 4596-4600.

For a review of chiral compounds of formula (II), including examples of their synthesis and their applications, see D. Seebach, A. K. Beck and A. Heckel, Angew. Chem. Int. Ed., 2001, 40, 92-138.

Synthetic routes to compounds of formula (III) are exemplified in: Y. Jia et al., Mol. Catal., 2020, 495, 111146; R. Custelcean and M. D. Ward, Angew. Chem. Int. Ed., 2002, 41, 10, 1724-1728; D. J. Ritson, R. J. Cox and J. Berge, Org. Biomol. Chem., 2004, 2, 1921-1933; J. L. Paih et al., J. Am. Chem. Soc., 2003, 125, 11964-11975; Y. Hu et al., J. Am. Chem. Soc., 1996, 118, 4550-4559; and M. Bamford et al., J. Med. Chem., 1995, 38, 3502-3513.

In some embodiments, the chiral compound of structure (Ib) is 5,5′,6,6′,7,7′,8,8′-octahydro-1,1′-bi-2-naphthol, i.e. of the following structure:

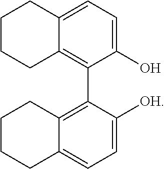 [SMILES: Oc1ccc2c(c1-c1c(O)ccc3c1CCCC3)CCCC2]

In some embodiments, the chiral compound of structure (IV) is 1,1′-spirobiindane-7,7′-diol, i.e. of the following structure:

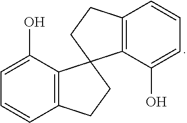 [SMILES: Oc1cccc2c1C1(CC2)CCc2cccc(O)c21]

In some embodiments the chiral compound is an atropisomeric biaryl compound. Often, the chiral compound is an atropisomeric biaryl compound of formula (I), (Ib) or (IV), such as (I). Sometimes, the atropisomeric biaryl compound is any one selected from the group consisting of [1,1′-binaphthalene]-2,2′-diol (BINOL); 6,6′-dibromo-1,1′-bi-2-naphthol; 2-amino-2′-hydroxy-1,1′-binaphthalene (NOBIN); 5,5′,6,6′,7,7′,8,8′-octahydro-1,1′-bi-2-naphthol; and 1,1′-spirobiindane-7,7′-diol. Typically, the atropisomeric biaryl compound is BINOL.

In some embodiments, the method of the invention further comprises isolating the tertiary or quaternary ammonium salt as a ternary complex comprising a tertiary or quaternary ammonium cation, anion X⁻ and the chiral compound. The skilled person is aware of techniques that are suitable for isolation of such ternary complexes. When solvents of low-polarity, i.e. those with a dielectric constant at 25° C. of ≤10, such as chloroform, are used, the ternary complex often precipitates out of solution and may be isolated by filtration techniques, such as vacuum filtration. To encourage precipitation, the volume of the solution may be reduced, e.g. by rotary evaporation, the temperature of the solution may be lowered, e.g. by refrigeration of the solution, and/or an anti-solvent may be used (in which the ternary complex is less soluble than the solvent already present). A suitable anti-solvent is miscible with the solvent already present in solution. As the solvent and anti-solvent mix, precipitation of the ternary complex is encouraged.

As described above, the inventors have found that recrystallising the ternary complex significantly increases the degree of enantioenrichment. Accordingly, in some embodiments, the method of the invention further comprises recrystallizing the ternary complex to form a recrystallised ternary complex. The skilled person is aware of techniques that are suitable for recrystallisation of the ternary complex. For example, the ternary complex may be dissolved in the minimum amount of solvent at a particular temperature (e.g. at ambient temperature (e.g. 15 to 25° C.) or at elevated temperatures where heat is applied to the solution) and the resultant solution cooled to encourage precipitation. Alternatively, or in addition, the volume of the solution may be reduced to encourage precipitation, e.g. by simple evaporation at ambient temperature and pressure. Alternatively, or in addition, an anti-solvent may be used (in which the ternary complex is less soluble than the solvent already present). A suitable solvent for recrystallisation of the ternary complex is one of high polarity, i.e. one with a dielectric constant at 25° C. of ≥17, such as methanol or ethanol. A suitable anti-solvent is one of low-polarity, i.e. one with a dielectric constant at 25° C. of ≤10, such as chloroform.

In some embodiments, the method of the invention further comprises isolating the tertiary or quaternary ammonium salt as an isolated tertiary or quaternary ammonium salt comprising a tertiary or quaternary ammonium cation and an anion X⁻. The tertiary or quaternary ammonium salt is typically isolated from the chiral compound by dissolving isolated or recrystallised ternary complex into a high polarity solvent, typically one with a dielectric constant at 25° C. of ≥17 and ≤40, which is miscible with water, such as methanol or ethanol. Water and a low polarity solvent with a dielectric constant at 25° C. of ≤10, which is not miscible with water, such as diethyl ether, are added to the solution.

Since the low polarity solvent is not miscible with water, the resultant solution comprises two phases—a water/high polarity solvent phase and a low polarity solvent phase. The tertiary or quaternary ammonium salt is typically more soluble in the water/high polarity solvent phase, whilst the chiral compound is typically more soluble in the low polarity solvent phase. Consequently, the tertiary or quaternary ammonium salt is recoverable by isolating the water/high polarity solvent phase, e.g. by separation, and concentrating to dryness. The chiral compound is also recoverable by isolating the low polarity solvent phase and concentrating this phase to dryness.

The skilled person is aware of separation techniques and routine adjustments that may be made in order more effectively to isolate the tertiary or quaternary ammonium salt. For example, rigorous mixing of the water/high polarity solvent phase and low polarity solvent phase promotes diffusion of the tertiary or quaternary ammonium salt into the water/high polarity solvent and the chiral compound into the low polarity solvent, leading to better isolation of the tertiary or quaternary ammonium salt. In addition, washing the low polarity solvent phase with water one or more times, and separating and concentrating the resultant aqueous phases to dryness leads to greater recovery of the tertiary or quaternary ammonium salt and a higher overall yield.

Isolated ternary complexes, recrystallized ternary complexes and some isolated tertiary or quaternary ammonium salts are stable and may be stored as solids at ambient temperature, e.g. at about 20° C., in the air. They may, but need not be, stored under inert conditions, e.g. under nitrogen or argon, or at reduced temperatures, e.g. in a refrigerator or freezer. Some isolated tertiary or quaternary ammonium salts deliquesce in air. Isolated ammonium salts may advantageously be stored under dry conditions, such as in sealed containers or in a desiccator.

In some embodiments, the method of the invention further comprises exchanging anion X⁻ of the isolated tertiary or quaternary ammonium salt for a different anion. The different anion is typically selected from the group consisting of [PF₆]⁻, [BF₄]⁻, [ClO₄]⁻, [B(C₆F₅)₄]⁻, [B(3,5-(CF₃)₂C₆H₃)₄]⁻, ⁻OTf, F⁻, Cl⁻, Br⁻, I⁻, ⁻OH, ⁻OTs, ⁻OAc, [H₂PO₄]⁻, [HSO₄]⁻ and [CH₃SO₃]⁻. For the avoidance of doubt, where X⁻ is one of the anions listed, this anion cannot be the different anion. For example, where X⁻ is Br, the different anion cannot be Br but may any one of the other anions listed. Often, the different anion is any one selected from the group consisting of [PF₆]⁻, [BF₄]⁻, [ClO₄]⁻, [B(C₆F₅)₄]⁻, [B(3,5-(CF₃)₂C₆H₃)₄]⁻, ⁻OH, ⁻OTs, ⁻OAc, [H₂PO₄]⁻, [HSO₄]⁻ and [CH₃SO₃]⁻.

Often, the different anion is one that is weakly coordinating, i.e. any one selected from the group consisting of [PF₆]⁻, [BF₄]⁻, [ClO₄]⁻, [B(C₆F₅)₄]⁻, and [B(3,5-(CF₃)₂C₆H₃)₄]⁻. The inventors have found that exchanging anion X⁻ of the isolated tertiary or quaternary ammonium salt for a weakly coordinating ion such as [PFs]-, renders the resultant tertiary or quaternary ammonium salt more stable in solution.

Without being bound by theory, reaction of the weakly coordinating ion with R of the tertiary or quaternary ammonium cation is highly unlikely, thereby avoiding re-formation of the tertiary amine from the tertiary or quaternary ammonium salt. It is for this reason that the tertiary or quaternary ammonium salt is understood to be highly stable in solution, and enantio-enrichment is retained.

The skilled person is aware of suitable techniques in the art for anion exchange. Often, the isolated tertiary or quaternary ammonium salt is reacted with the different anion in solution. Most often, the isolated tertiary or quaternary ammonium salt is dissolved in a suitable solvent, typically one of high polarity with a dielectric constant at 25° C. of ≥17 (e.g. water), and an excess of salt comprising the different anion is added to the resultant solution. The resultant tertiary or quaternary ammonium salt may be isolated from the solution, for example by extraction into a suitable solvent. Sometimes, the resultant tertiary or quaternary ammonium salt is extracted into a low polarity solvent with a dielectric constant at 25° C. of ≤10, which is not miscible with water, such as dichloromethane. The tertiary or quaternary ammonium salt is then isolated by concentrating the low polarity solvent to dryness, e.g. by rotary evaporation.

The second aspect of the invention provides for use of a non-racemic chiral compound in the synthesis of an enantiomerically enriched tertiary or quaternary ammonium salt from a tertiary amine, wherein the chiral compound has at least two substituents capable of coordinating to the tertiary or quaternary ammonium salt.

The relevant embodiments of the first aspect of the invention, i.e. those relating to a chiral compound that has at least two substituents capable of coordinating to the tertiary or quaternary ammonium salt, also apply to the second aspect. For example the chiral compound may comprise two —OH groups that are capable of coordinating to the tertiary or quaternary ammonium salt; the chiral compound may have any one of structures (I) to (III) defined above; the chiral compound may be an atropisomeric biaryl compound; and/or the chiral compound may be BINOL.

The use according to the second aspect of the non-racemic chiral compound is for the synthesis of an enantiomerically enriched tertiary or quaternary ammonium salt from a tertiary amine. The relevant embodiments of the first aspect of the invention, i.e. those relating to the synthesis of an enantiomerically enriched tertiary or quaternary ammonium salt from a tertiary amine, also apply to the second aspect. For example, the synthesis may comprise reacting a tertiary amine with a compound of formula R—X, wherein the tertiary amine is chiral at the nitrogen atom, R is different to any substituent on the nitrogen atom of the tertiary amine and X is a leaving group, and wherein the reacting is effected under reversible conditions in the presence of the non-racemic chiral compound. A ratio of tertiary amine to R—X of 1:≥2 may be used; a ratio of tertiary amine to chiral compound of 1:≥1 may be used; the tertiary amine may be of formula N(R¹)₃ as defined above; X may be bromo or iodo; the tertiary or quaternary ammonium salt may be isolated as a ternary complex; the ternary complex may be recrystallized; the tertiary or quaternary ammonium salt may be isolated as an isolated tertiary or quaternary ammonium salt; and/or the anion X⁻ of the isolated tertiary or quaternary ammonium salt may be exchanged for a different anion.

Each and every patent and non-patent reference referred to herein is hereby incorporated by reference in its entirety, as if the entire contents of each reference were set forth herein in their entirety.

Aspects and embodiments of the invention are further described in the following clauses:

1. A method of making an enantiomerically enriched tertiary or quaternary ammonium salt comprising reacting a tertiary amine with a compound of formula R—X to form a tertiary or quaternary ammonium salt, wherein the tertiary amine is chiral at the nitrogen atom, R is different to any substituent on the nitrogen atom of the tertiary amine and X is a leaving group and wherein the reacting is effected under reversible conditions in the presence of a non-racemic chiral compound having at least two substituents capable of coordinating to the tertiary or quaternary ammonium salt.

2. The method of clause 1, wherein the ratio of tertiary amine to R—X is any one selected from the group consisting of 1:>1, 1:≥1.2, 1:≥1.4, 1:≥1.6, 1:≥1.8 and 1:≥2.

3. The method of clause 1, wherein the ratio of tertiary amine to R—X is 1:≥2.

4. The method of any one preceding clause, wherein the ratio of tertiary amine to non-racemic chiral compound is any one selected from the group consisting of 1:>0.5, 1:≥0.6, 1:≥0.7, 1:≥0.8, 1:≥0.9 and 1:≥1.

5. The method of any one of clauses 1 to 3, wherein the ratio of tertiary amine to non-racemic chiral compound is 1:≥1.

6. The method of any one preceding clause, wherein the tertiary amine is of formula N(R¹)₃, wherein each R¹ is a different hydrocarbyl group optionally comprising one or more heteroatoms selected from the group consisting of oxygen, nitrogen, sulphur, fluorine, boron, bromine, chlorine, phosphorous and iodine.

7. The method of any one preceding clause, wherein R is a hydrocarbyl group which is different to each R¹.

8. The method of any one of clauses 1 to 5, wherein the tertiary amine is of formula N(R¹)₃, and each R¹ is independently selected from the group consisting of C₁-C₆alkyl, C₂-C₆alkenyl, C₂-C₆alkynyl, C₆-C₁₀aryl, C₆-C₂₄biaryl, C₆-C₁₀arylC₁-C₆alkyl, C₆-C₂₄biarylC₁-C₆alkyl, C₆-C₁₀arylacyl, C₆-C₂₄biarylacyl, C₃-C₃cycloalkyl, C₃-C₈cycloalkylC₁-C₆alkyl and C₃-C₅heteroaryl, optionally substituted with any one or a combination selected from the group consisting of hydroxy, oxo and amino; or
two R¹ groups together with the nitrogen atom to which they are attached form indolyl, tetrahydroquinolinyl, 3-azabicyclo[3.2.1]octanyl or camphidinyl, optionally substituted with any one or a combination selected from the group consisting of C₁-C₆alkyl, C₂-C₆alkenyl, C₂-C₆alkynyl, hydroxy, oxo and amino; morpholino, pyrrolidino or piperidinyl, substituted with any one or a combination selected from the group consisting of C₁-C₆alkyl, C₂-C₆alkenyl, C₂-C₆alkynyl, hydroxy, oxo and amino such that the resultant N(R¹)₂ is asymmetric; and the other R¹ group is selected from the group consisting of C₁-C₆alkyl, C₂-C₆alkenyl, C₂-C₆alkynyl, C₆-C₁₀aryl, C₆-C₂₄biaryl, C₆-C₁₀arylC₁-C₆alkyl, C₆-C₂₄biarylC₁-C₆alkyl, C₆-C₁₀arylacyl, C₆-C₂₄biarylacyl, C₃-C₈cycloalkyl and C₃-C₈cycloalkylC₁-C₆alkyl, optionally substituted one or more times with any one or a combination selected from the group consisting of hydroxy, oxo and amino; or
all three R¹ groups together with the nitrogen atom to which they are attached form 1,4-diazabicyclo[2.2.2]octane or 1-azabicyclo[2.2.2]octane substituted with any one or a combination selected from the group consisting of C₁-C₆alkyl, C₂-C₆alkenyl, C₂-C₆alkynyl, hydroxy, oxo and amino such that the resultant N(R¹)₃ is chiral; or
N(R¹)₃ is morphine, nalorphine, naltrexone, oxymorphone, or atropine.

9. The method of any one of clauses 1 to 5, wherein the tertiary amine is of formula N(R¹)₃, and each R¹ is independently selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted with any one or a combination selected from the group consisting of hydroxy, oxo and amino; or
two R¹ groups together with the nitrogen atom to which they are attached form indolyl, tetrahydroquinolinyl, 3-azabicyclo[3.2.1]octanyl or camphidinyl, optionally substituted with any one or a combination selected from the group consisting of C₁-C₆alkyl, C₂-C₆alkenyl, C₂-C₆alkynyl, hydroxy, oxo and amino; morpholino, pyrrolidino or piperidinyl, substituted with any one or a combination selected from the group consisting of C₁-C₆alkyl, C₂-C₆alkenyl, C₂-C₆alkynyl, hydroxy, oxo and amino such that the resultant N(R¹)₂ is asymmetric; and the other R¹ is selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted one or more times with any one or a combination selected from the group consisting of hydroxy, oxo and amino; or
all three R¹ groups together with the nitrogen atom to which they are attached form 1,4-diazabicyclo[2.2.2]octane or 1-azabicyclo[2.2.2]octane substituted with any one or a combination selected from the group consisting of C₁-C₆alkyl, C₂-C₆alkenyl, C₂-C₆alkynyl, hydroxy, oxo and amino such that the resultant N(R¹)₃ is chiral; or
N(R¹)₃ is morphine, nalorphine, naltrexone, oxymorphone, or atropine.

10. The method of any one of clauses 1 to 5, wherein the tertiary amine is of formula N(R¹)₃, and each R¹ is independently selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted with one or more hydroxy and/or amino; or
two R¹ groups together with the nitrogen atom to which they are attached form indolyl, tetrahydroquinolinyl, 3-azabicyclo[3.2.1]octanyl or camphidinyl, optionally substituted with any one or a combination selected from the group consisting of C₁-C₆alkyl, C₂-C₆alkenyl, C₂-C₆alkynyl, hydroxy and amino; and the other R¹ group is selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted with one or more hydroxy and/or amino; or
N(R¹)₃ is morphine, nalorphine, naltrexone, oxymorphone, or atropine.

11. The method of any one of clauses 1 to 5, wherein the tertiary amine is of formula N(R¹)₃, and each R¹ is independently selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl; or
two R¹ groups together with the nitrogen atom to which they are attached form indolyl, tetrahydroquinolinyl, 3-azabicyclo[3.2.1]octanyl or camphidinyl, optionally substituted with any one or a combination selected from the group consisting of C₁-C₆alkyl, C₂-C₆alkenyl, C₂-C₆alkynyl, hydroxy and amino; and the other R¹ group is selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl; or
N(R¹)₃ is morphine, nalorphine, naltrexone, oxymorphone, or atropine.

12. The method of any one preceding clause, wherein R is selected from the group consisting of C₁-C₆alkyl, C₂-C₆alkenyl, C₂-C₆alkynyl, C₆-C₁₀aryl, C₆-C₂₄biaryl, C₆-C₁₀arylC₁-C₆alkyl, C₆-C₂₄biarylC₁-C₆alkyl and C₃-C₃cycloalkyl.

13. The method of any one of clauses 1 to 11, wherein R is selected from the group consisting of C₁-C₆alkyl, C₂-C₆alkenyl, C₂-C₆alkynyl, C₆-C₁₀aryl, C₆-C₁₀arylC₁-C₆alkyl and C₃-C₈cycloalkyl.

14. The method of any one of clauses 8 to 13, wherein C₁-C₆alkyl is C₁-C₄alkyl.

15. The method of any one of clauses 8 to 14, wherein C₂-C₆alkenyl is C₂-C₄alkenyl.

16. The method of any one of clauses 8 to 15, wherein C₂-C₆alkynyl is C₂-C₄alkynyl.

17. The method of any one of clauses 8 to 16, wherein C₆-C₁₀aryl is phenyl.

18. The method of any one of clauses 8 to 17, wherein C₆-C₁₀arylC₁-C₆alkyl is phenylC₁-C₆alkyl.

19. The method of clause 18, wherein phenylC₁-C₆alkyl is benzyl.

20. The method of any one preceding clause, wherein each substituent on the tertiary amine has a different Taft steric substituent constant (Eₛ) and the Taft steric substituent constants differ by >0.07.

21. The method of any one preceding clause, wherein X is selected from the group consisting of halo, triflate, tosylate, phosphate and acetoxy.

22. The method of clause 21, wherein halo is bromo or iodo.

23. The method of any one of clauses 1 to 20, wherein X is bromo or iodo.

24. The method of any one preceding clause, wherein the at least two substituents capable of coordinating to the tertiary or quaternary ammonium salt are each —OH.

25. The method of any one preceding clause, wherein the chiral compound has two substituents capable of coordinating to the tertiary or quaternary ammonium salt.

26. The method of any one of clauses 1 to 23, wherein the chiral compound is any one of structures (I), (Ib), (II), (III) or (IV):

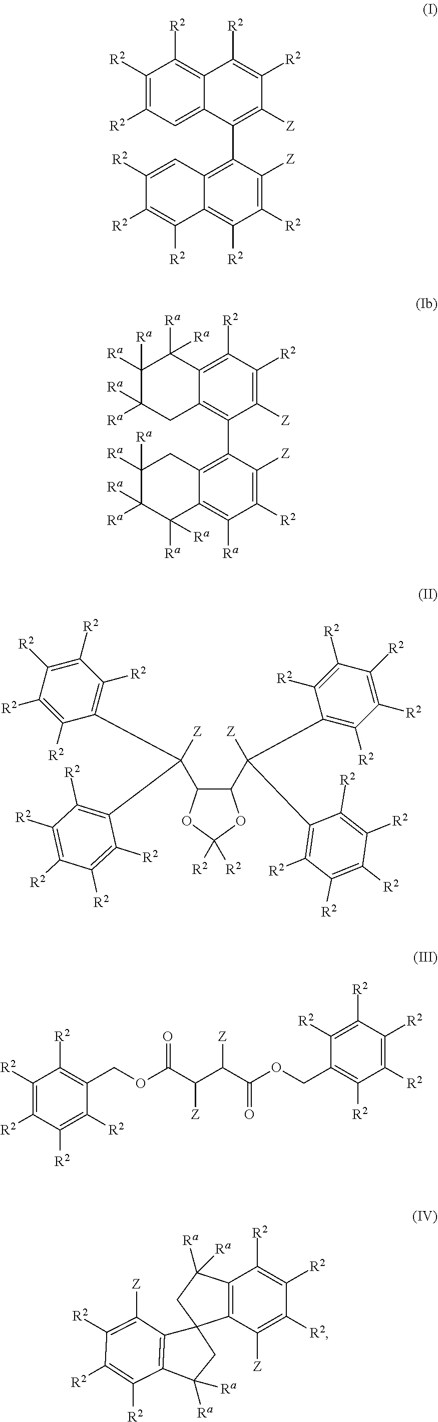

wherein each R² is independently selected from the group consisting of —H, halo, C₁-C₆alkyl, C₂-C₆alkenyl, C₂-C₆alkynyl, C₆-C₁₀aryl, C₆-C₁₀arylC₁-C₆alkyl, C₁-C₆-mono-, di- or tri-alkylC₆-C₁₀aryl, C₁-C₆mono-, di- or tri-fluoroalkylC₆-C₁₀aryl, tri-C₆-C₁₀arylsilyl, C₃-C₈cycloalkyl, C₃-C₈cycloalkylC₁-C₆alkyl, C₁-C₆mono-, di- or tri-alkylC₃-C₈cycloalkyl, C₁-C₆mono-, di- or tri-fluoroalkylC₃-C₈cycloalkyl; and
each R^{a} is independently selected from the group consisting of —H, C₁-C₆alkyl, C₂-C₆alkenyl, C₂-C₆alkynyl, C₆-C₁₀aryl, C₆-C₁₀arylC₁-C₆alkyl, C₁-C₆mono-, di- or tri-alkylC₆-C₁₀aryl, C₁-C₆mono-, di- or tri-fluoroalkylC₆-C₁₀aryl, tri-C₆-C₁₀arylsilyl, C₃-C₈cycloalkyl, C₃-C₈cycloalkylC₁-C₆alkyl, C₁-C₆mono-, di- or tri-alkylC₃-C₈cycloalkyl, C₁-C₆mono-, di- or tri-fluoroalkylC₃-C₈cycloalkyl.

27. The method of any one of clauses 1 to 23, wherein the chiral compound is any one of structures (1) to (III):

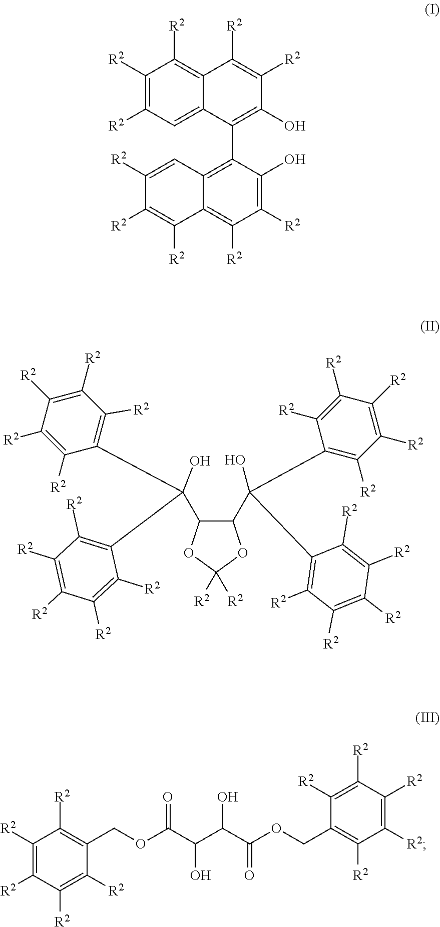

wherein each R² is independently selected from the group consisting of —H, halo, C₁-C₆alkyl, C₂-C₆alkenyl, C₂-C₆alkynyl, C₆-C₁₀aryl, C₆-C₁₀arylC₁-C₆alkyl, C₁-C₆-mono-, di- or tri-alkylC₆-C₁₀aryl, C₁-C₆mono-, di- or tri-fluoroalkylC₆-C₁₀aryl, tri-C₆-C₁₀arylsilyl, C₃-C₈cycloalkyl, C₃-C₈cycloalkylC₁-C₆alkyl, C₁-C₆mono-, di- or tri-alkylC₃-C₈cycloalkyl, C₁-C₆mono-, di- or tri-fluoroalkylC₃-C₈cycloalkyl.

28. The method of clause 26 or 27, wherein each R² is independently selected from the group consisting of —H, C₁-C₆alkyl, C₂-C₆alkenyl, C₂-C₆alkynyl, C₆-C₁₀aryl, C₆-C₁₀arylC₁-C₆alkyl, C₁-C₆mono-, di- or tri-alkylC₆-C₁₀aryl, C₁-C₆mono-, di- or tri-fluoroalkylC₆-C₁₀aryl, tri-C₆-C₁₀arylsilyl, C₃-C₈cycloalkyl, C₃-C₈cycloalkylC₁-C₆alkyl, C₁-C₆mono-, di- or tri-alkylC₃-C₈cycloalkyl, C₁-C₆mono-, di- or tri-fluoroalkylC₃-C₈cycloalkyl.

29. The method of clause 26 or 27, wherein each R² is independently selected from the group consisting of —H, C₁-C₆alkyl, C₆-C₁₀aryl, C₁-C₆tri-alkylC₆-C₁₀aryl, C₁-C₆di-fluoroalkylC₆-C₁₀aryl, tri-C₆-C₁₀arylsilyl and C₃-C₈cycloalkyl.

30. The method of clause 26 or 27, wherein each R² is independently selected from the group consisting of —H, tri-isopropylphenyl, di-trifluoromethylphenyl and triphenylsilyl.

31. The method of clause 26 or 27, wherein each R² is —H.

32. The method of any one preceding clause, wherein the chiral compound is an atropisomeric biaryl compound.

33. The method of any one of clauses 1 to 23, wherein the chiral compound is any one selected from the group consisting of [1,1′-binaphthalene]-2,2′-diol, 6,6′-dibromo-1,1′-bi-2-naphthol; 2-amino-2′-hydroxy-1,1′-binaphthalene; 5,5′,6,6′,7,7′,8,8′-octahydro-1,1′-bi-2-naphthol; and 1,1′-spirobiindane-7,7′-diol.

34. The method of any one of clauses 1 to 23, wherein the chiral compound is [1,1′-binaphthalene]-2,2′-diol.

35. The method of any one preceding clause, further comprising isolating the tertiary or quaternary ammonium salt as a ternary complex comprising a tertiary or quaternary ammonium cation, anion X⁻ and chiral compound.

36. The method of clause 35, further comprising recrystallizing the ternary complex to form a recrystallised ternary complex.

37. The method of clause 35 or clause 36, further comprising isolating the tertiary or quaternary ammonium salt as an isolated tertiary or quaternary ammonium salt comprising a tertiary or quaternary ammonium cation and an anion X⁻.

38. The method of clause 37, further comprising exchanging anion X⁻ for a different anion selected from the group consisting of [PF₆]⁻, [BF₄]⁻, [ClO₄]⁻, [B(C₆F₅)₄]⁻, [B(3,5-(CF₃)₂C₆H₃)₄]⁻, —OTf, F⁻, Cl⁻, Br⁻, I⁻, ⁻OH, ⁻OTs, ⁻OAc, [H₂PO₄]⁻, [HSO₄]⁻ and [CH₃SO₃]⁻.

39. Use of a non-racemic chiral compound in the synthesis of an enantiomerically enriched tertiary or quaternary ammonium salt from a tertiary amine, wherein the chiral compound has at least two substitutents capable of coordinating to the tertiary or quaternary ammonium salt.

40. The use of clause 39, wherein the synthesis is according to the method of any one of clauses 1 to 23 and 35 to 38.

41. The use of clause 39 or clause 40, wherein the chiral compound is as disclosed in any one of clauses 24 to 34.

The following non-limiting examples below serve to illustrate the invention further.

### EXAMPLES

### General Experimental Procedures

All reagents and solvents are commercially available (Sigma Aldrich, Fischer, Fluorochem, and TCI) and were used without further purification, unless stated otherwise. Reactions were carried out without drying, open to atmosphere, unless stated otherwise. Anhydrous solvents (dichloromethane, toluene) were obtained by following standard distillation over P₂O₅ and stored under N₂. Room temperature (r.t.) reactions were carried out at between 15-25° C. Column chromatography was typically carried out using 60 Å (70-230 mesh) silica gel from VWR (see C. W. Still et al., J. Org. Chem., 43, 2923-2925 (1978)). Thin layer chromatography (TLC) was conducted using 2 cm×5 cm aluminium backed plates coated with a silica matrix (0.2 mm) and fluorescent indicator (254 nm). Visualisation of TLC plates was carried out using a UV lamp and/or appropriate staining (Ninhydrin, phosphomolybdic acid (PMA), see Michael C. Pirrung, The Synthetic Organic Chemist's Companion, John Wiley & Sons, 171-172 (2007)).

NMR spectra were recorded on either a Bruker Avance Ill-HD-400 spectrometer with operating frequencies of 400.07 MHz for ¹H, 100.60 MHz for ¹³C, 376.45 MHz for ¹⁹F, 161.95 MHz for ³¹P, or a Varian VNMRS-600 spectrometer with operating frequencies of 599.42 MHz for ¹H, 150.72 MHz for ¹³C, 564.02 MHz for ¹⁹F, 242.65 MHz for ³¹P, at 298 K. Spectra were processed using MestReNova (V. 12.0) software. ¹H NMR Chemical shifts were referenced to residual non-deuterated solvent peaks within the NMR solvent; CHCl₃ (δH=7.26 ppm), CH₃CN (δH=1.94 ppm), CH₃OH (δH=3.31 ppm) DMSO (δH=2.50 ppm), see H. E. Gottlieb, V. Kotlyar, A. J. Nudelman, J. Org. Chem., 62, 7512-7515 (1997). The multiplicity of ¹H NMR signals are indicated as: s=singlet; d=doublet; t=triplet; q=quartet; quint.=quintet; sex.=sextet; sept.=septet; m=multiplet; br=broad; and combinations thereof. Coupling constants (J) are quoted in Hz and reported to the nearest 0.1 Hz. Chemical shifts for ¹³C NMR spectra were referenced to deuterated solvent peaks in the NMR solvent; CDCl₃ (δC═77.16 ppm), CD₃CN (δC=1.32 ppm), CD₃OD (δC=49.00 ppm) DMSO-d₆ (δC=39.52 ppm), see H. E. Gottlieb, V. Kotlyar, A. J. Nudelman (supra). All ¹³C NMR signals are reported to the nearest 0.1 ppm in general, or to 0.01 ppm to aid in the differentiation of closely resolved signals. Product identification and peak assignments were completed using 2D experiments (correlated spectroscopy (COSY), heteronuclear single quantum coherence (HSQC) spectroscopy, heteronuclear multiple bond correlation (HMBC) spectroscopy) and pulse experiments (distortionless enhancement by polarisation transfer (DEPT-135)) where appropriate.

All mass spectrometry was carried out using a tandem Acquity ultra performance liquid chromatography (UPLC) (Waters Ltd, UK) and a triple quadrupole detector (TQD) with electron spray iononisation (ESI) mass spectrometer (set to EI+mode and EI-mode where appropriate). The UPLC was equipped with an Acquity UPLC BEH C18 1.7 mm (2.1 mm×50 mm) column, and mobile phase composition of H₂O containing formic acid (0.1% v/v): Methanol mobile phase (gradient elution; t=0 min, 95%: 0%, t=4 min, 5%: 95%), set at 0.6 mL·min⁻¹.

Melting points of solid and crystalline products were measured using a Sanyo Gallenkamp variable heater equipped with a 300° C. mercury thermometer. Melting points are uncorrected and solvents of crystallisation are listed along with the observed melting point range where appropriate.

Infra-red (IR) spectra were acquired using a Perkin Elmer Spectrum 100 fourier transform (FT) IR spectrometer equipped with a universal attenuated total reflectance (UATR) attachment and CsI window. Spectra were recorded from a range of 4,000-380 cm⁻¹. Absorbance shape and intensity are described as w=weak; m=medium; s=strong; sh=sharp; br=broad.

Optical rotation measurements (a) were conducted on a Schmidt & Haensch UniPol L2000 polarimeter, equipped with a 589.44 nm Na light source. The temperature was controlled using a Brookfield TC-550MX circulating water bath, and a jacketed 100 mm quartz cell. Samples were prepared using HPLC grade solvents.

Rotation measurements were repeated in quintuplicate and are reported as an average specific rotation ([α]D), along with the concentration (c) in M, and solvent used for the measurement.

X-ray single crystal data was collected using λMoKα radiation (λ=0.71073 Å) on an Agilent XCalibur (Sapphire-3 charge-coupled device (CCD) detector, fine-focus sealed tube, graphite monochromator; compounds (rac)-1b and 2c) and Bruker D8 Venture (Photon100 complementary metal-oxide semiconductor (CMOS) detector, I μS-microsource, focusing mirrors; all other compounds) diffractometers equipped with Cryostream (Oxford Cryosystems) open-flow nitrogen cryostats at a temperature of 120.0 K. All structures were solved by direct methods and refined by full-matrix least squares on F2 for all data using Olex2 (see O. V. Dolomanov et al., J. Appl. Cryst., 42, 339-341 (2009)) and SHELXTL (see G. M. Sheldrick, Acta Cryst., A 71, 3-8 (2015) and G. M. Sheldrick et al., J. Appl. Cryst., 44, 1281-1284 (2011)) software. All non-disordered non-hydrogen atoms were refined anisotropically, the hydrogen atoms were placed in the calculated positions and refined in riding mode. Crystallographic data and related crystallographic information files (CIFs) for the all structures have been deposited with the Cambridge Crystallographic Data Centre as supplementary publications: CCDC-1987042-1987058; 1987061-1987068; 1987165-1987180.

### Results

### Enantioselective Recognition

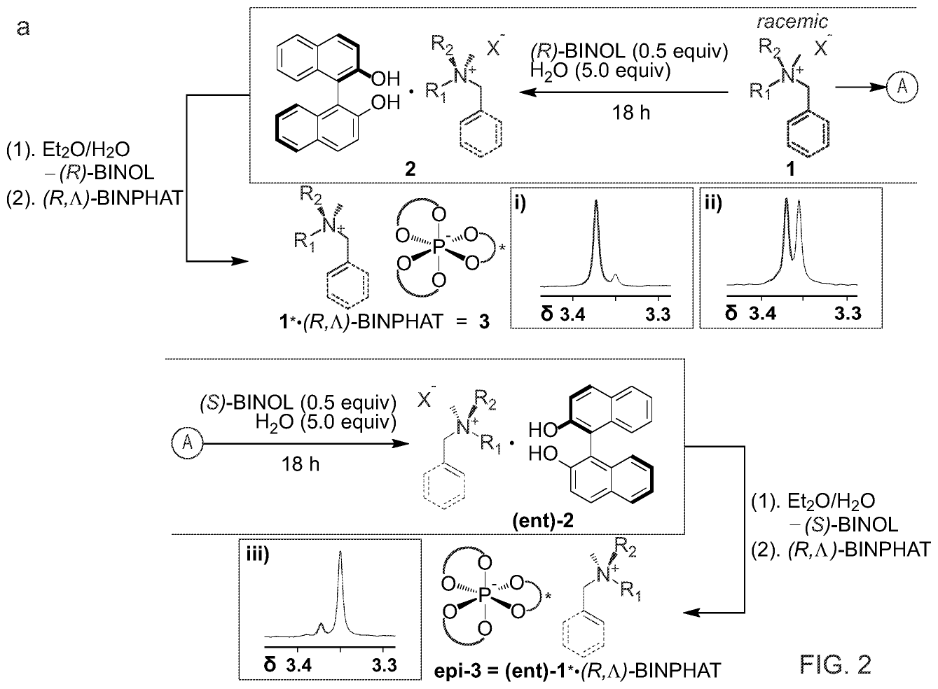

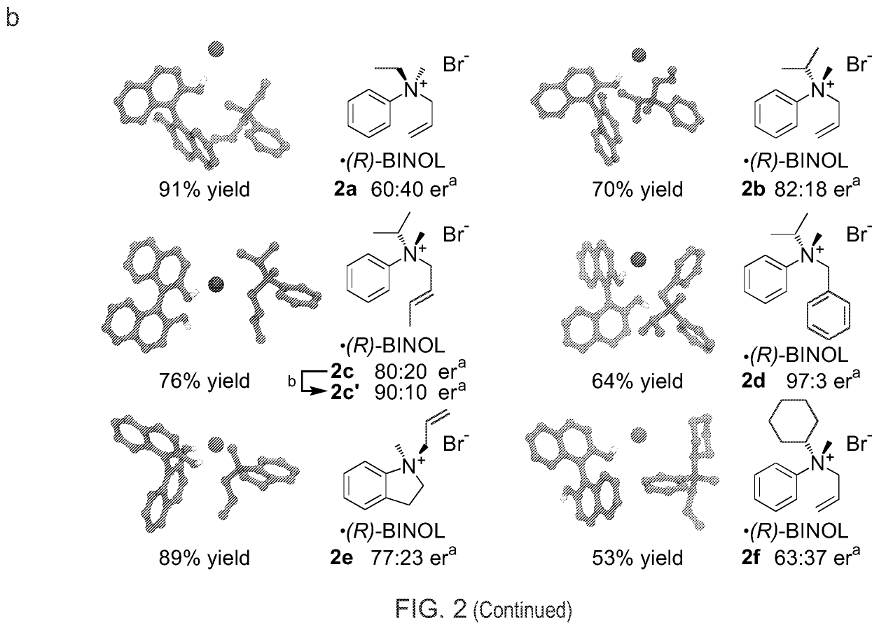

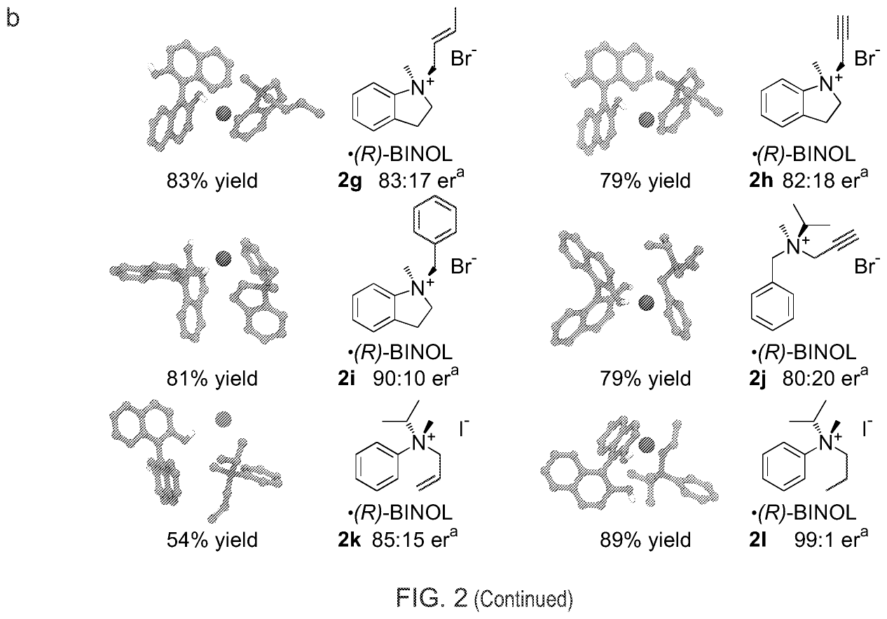

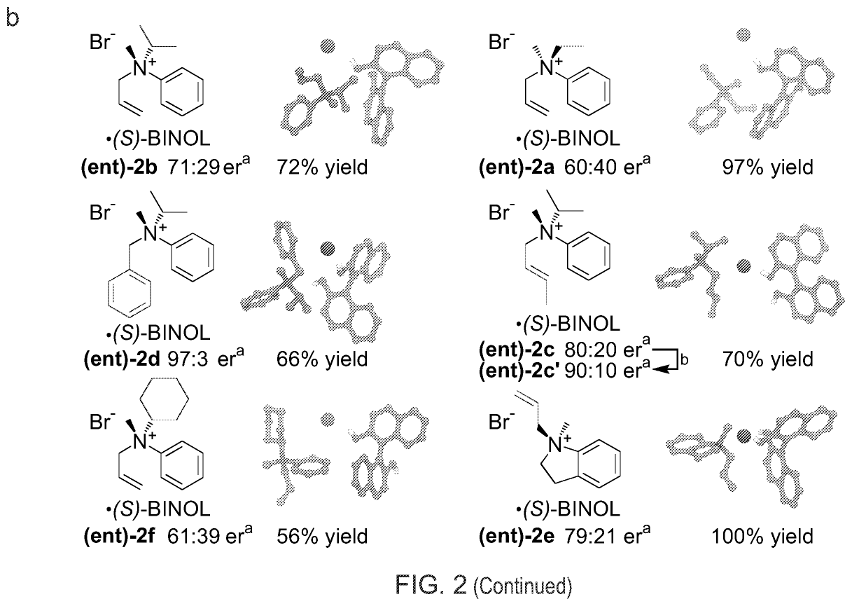

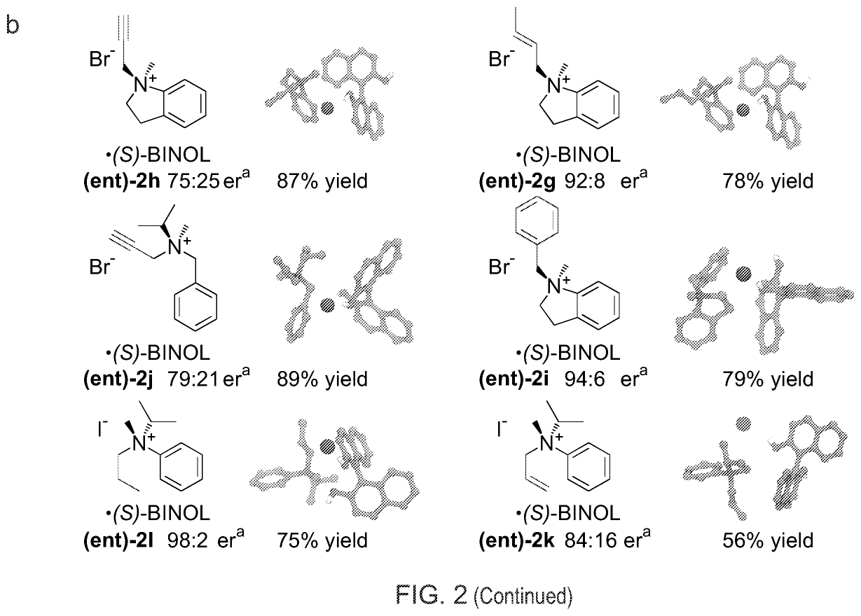

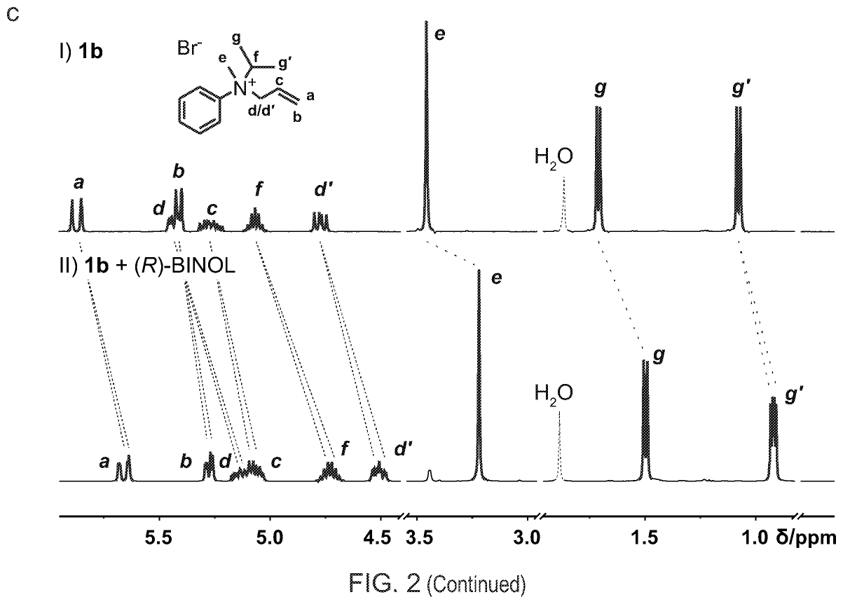

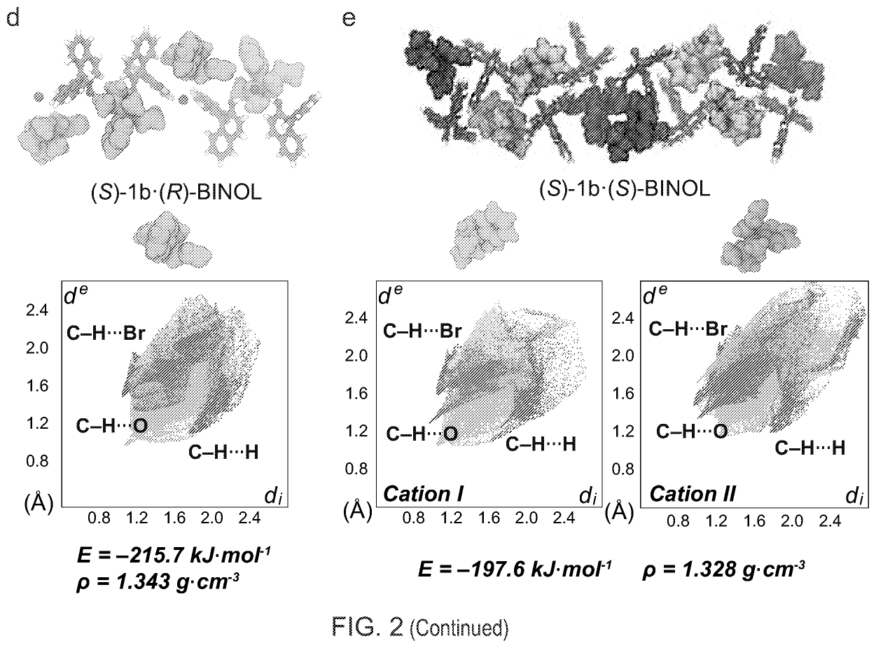

Initial investigations demonstrated that BINOL.X-.quaternary ammonium cation complex formation enables the resolution of chiral quaternary ammonium salt species without an additional recognition handle (FIG. 2). Addition of (R)-BINOL (0.5 equiv) to a variety of racemic quaternary ammonium salts under concentrated conditions (0.6 M) rapidly generated an enantioenriched ternary complex (2 in FIG. 2a), which precipitated from solution. The enantiopurity of the ternary complex was quantified by ¹H NMR analysis, employing the NMR chiral shift reagent (R, Λ)-BINPHAT (see J. Lacour, L. Vial, & C. Herse, Org. Lett. 4, 1351-1354 (2002)). A portion of the isolated material was de-complexed from BINOL by aqueous extraction (Et₂O/H₂O). The NMR chiral shift reagent is then able to coordinate to the quaternary ammonium salt, forming diastereomeric salt 3 in FIG. 2a). The absolute configuration of each sample was confirmed by X-ray diffraction (FIG. 2b).

Comparing the degree of enantioenrichment exhibited by quaternary ammonium salts 1a and 1b shows that a larger difference in steric bulk between the substituents at the chiral nitrogen centre gives rise to an increased enantioenrichment. While ethyl ammonium 1a provided ternary complex 2a in good yield, low levels of enantioenrichment were observed (FIG. 2b. entry 2a, 60:40 er). In contrast, isopropyl ammonium 1b, exhibited higher levels of enantiomeric purity (FIG. 2b. entry 2b, 82:18 er).

The degree of enantioenrichment could be improved through recrystallisation of the ternary complex. For example, complexation of 1c initially yielded complex 2c (80:20 er) which was enhanced with a single recrystallisation providing 2c′ with higher levels of enrichment (90:10 er).

A wide variety of anilinium (2a-d, 2f, 2k-l), indolinium (2e, 2g-i) and benzyl ammonium (2j) cores were readily resolvable in good to excellent yields (53-89%) with levels of enrichment from modest (2f, 63:37 er) to excellent (2l, 99:1 er). Isolated yields were calculated based on the equivalences of BINOL used. Isolation of the opposite enantiomeric form of each quaternary ammonium salt was achieved with similar yields and enantioselectivies by simply employing the opposite enantiomer of BINOL (FIG. 2b, (ent)-2a-(ent)-2k).

Experimental evidence indicates that the mechanism of ammonium recognition has both a solution and solid phase component. On addition of BINOL (0.5 equiv) to a sample of 1b in chloroform, distinctive shifts were observed in the ¹H NMR spectrum (FIG. 2. c). NMR signals corresponding to aliphatic proton environments adjacent to the quaternary ammonium centre (FIG. 2. c. II, signals Ha-Hg) showed both an increase in multiplicity and significant upfield shifts, consistent with binding of the BINOL to the quaternary ammonium salt in solution, forming a mixture of two diastereomeric complexes. Coordination of BINOL to a quaternary ammonium salt that is chiral at the nitrogen centre favours one enantiomer over the other. To understand the differences between the ternary complexes comprising BINOL and the favoured quaternary ammonium salt enantiomer and BINOL and the disfavoured quaternary ammonium salt enantiomer, an example of each ternary complex was prepared. The complex resultant from treatment of racemic 1b with (R)-BINOL (ternary complex 2b), was recrystallised to high enantiomeric enrichment, affording the matched pair (S)-1b.(R)-BINOL. A portion of 2b was treated to remove the BINOL by extraction. The recovered enriched (S)-1b was then complexed with (S)-BINOL yielding the unfavoured diastereomer (S)-1b.(S)-BINOL (the mismatched pair). The crystal structure of each diastereomer was analysed (FIG. 2. d & e) and globally represented as a 2D surface and portrayed as Hirshfeld fingerprint plots (see M. A. Spackman and D. Jayatilaka, Cryst. Eng. Comm., 11, 19-32 (2009)). The Hirshfeld plot of the mismatched pair (Z′=2), is more diffuse, indicative of less efficient packing. In contrast, the Hirshfield plot of the matched pair (Z′=1), is more compact, indicative of more efficient packing within the unit cell of the crystal. Melting point analysis also indicates a greater stability of the matched (mp=150-152° C.) than the mismatched (mp=137-139° C.) pair. These results are consistent with a solution phase recognition of the quaternary ammonium salt through formation of BINOL.X-.quaternary ammonium cation ternary complexes, which subsequently act as a nucleation centre, and allow for selective abstraction to the solid phase through an adductive crystallisation (see J. W. Mullin, Ullman's Encyclopedia of Industrial Chemistry Vol. 10 (ed. Barbara Elvers), 582-300, 630 (Wiley-VCH, 2012)).

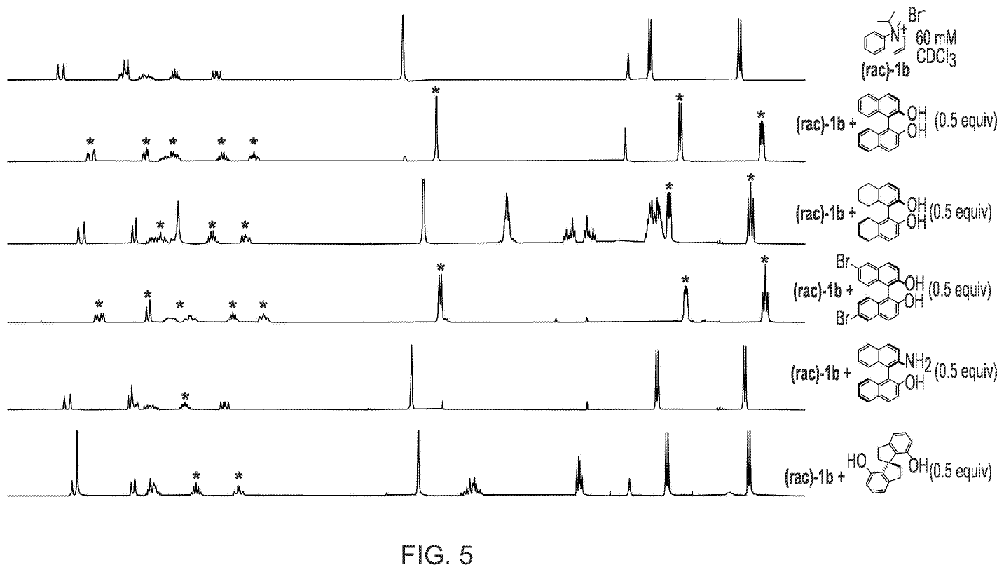

The solution state enantioselective recognition of 1b by a variety of non-racemic chiral compounds other than BINOL is demonstrated by the ¹H NMR signals labelled with * in FIG. 5.

### Racemisation of the Quaternary Ammonium Stereocentre

Some conditions suitable for the racemisation of the quaternary ammonium stereocentre have been established. It was found that certain quaternary ammonium salts are dissociative at room temperature, in chloroform. This dissociative behaviour is dependent on the solvent in which the quaternary ammonium salt is dissolved (see Table 1, below). Dissociation occurs in non-polar, aprotic solvents but is inhibited in polar, protic solvents. Consequently, an enantioenriched ammonium salt should be stable in polar, protic solvents such as methanol, even under prolonged heating.

TABLE 1
Dynamic behaviour of 155 at 50° C. in acetonitrile and methanol.

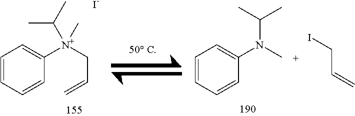

| Time/h | Solvent | Concentration/mM | Ratio (155:190) |
|---|---|---|---|
| 18 | MeCN | 60 | 60:40 |
| 18 | MeOH | 60 | 100:0 |

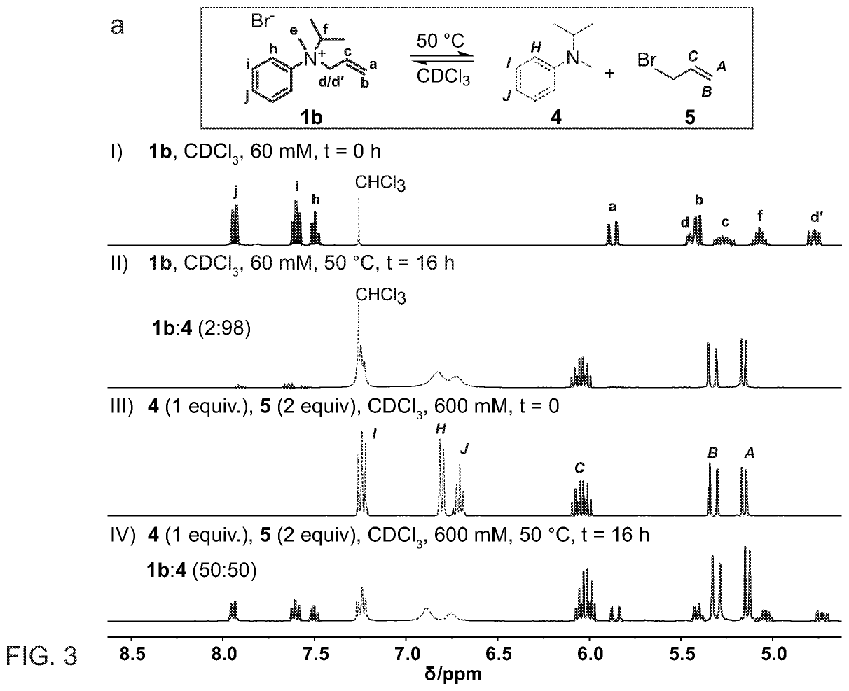

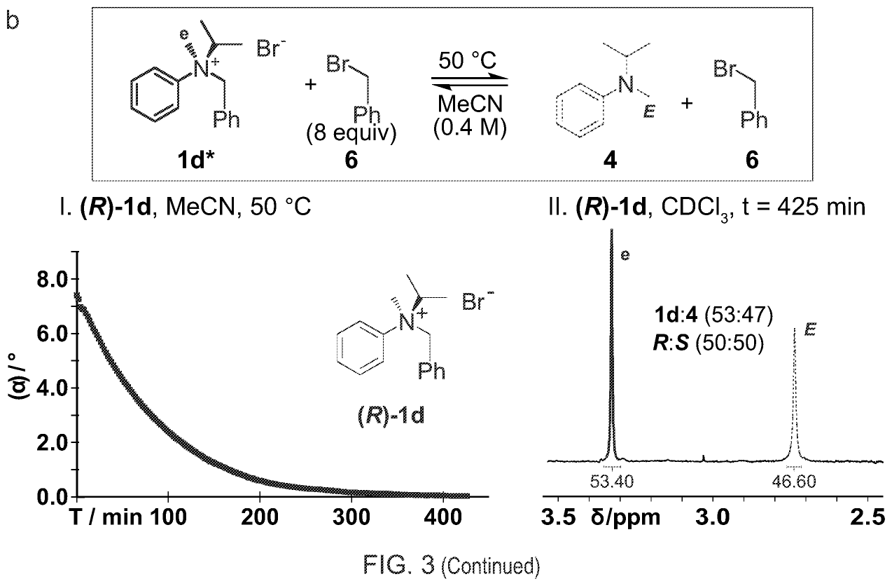

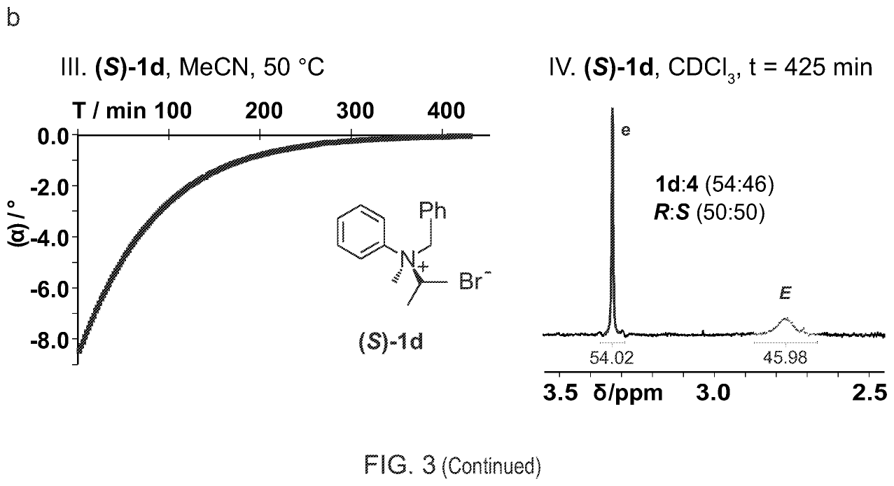

It was found that 1b (FIG. 3a. I) fully dissociates when heated at dilute concentrations affording aniline 4 and allyl bromide 5 (FIG. 3a. II). At higher concentrations, the position of equilibrium is biased in favour of the ammonium salt. When 4 and 5 were heated at 50° C., (FIG. 3a. III), the ratio of 4 to 1b at equilibrium was determined to be equal by ¹H NMR (FIG. 3a. IV. 4:1b, 50:50). To observe the stereochemical integrity of the ammonium directly, a sample of enriched (R)-1d was dissolved in acetonitrile (0.4 M) with an excess (8 equiv) of benzyl bromide 6 and heated. Analysis of the rate of decay of optical activity demonstrated that chiral information was eroded under pseudo first order kinetics (FIG. 3b. I. t_{1/2}=56.5 min, λ=0.012) with complete loss of optical activity after 425 min. (S)-1d showed identical behaviour (FIG. 3b. III). ¹H NMR analysis of the end points of these measurements showed significant quantities of quaternary ammonium salt remaining in solution, confirming that the loss in optical activity was due to racemisation of the nitrogen centre rather than simple dissociation (FIG. 3b. II & IV).

### Synthesis of Enantio-Enriched Quaternary Ammonium Salts

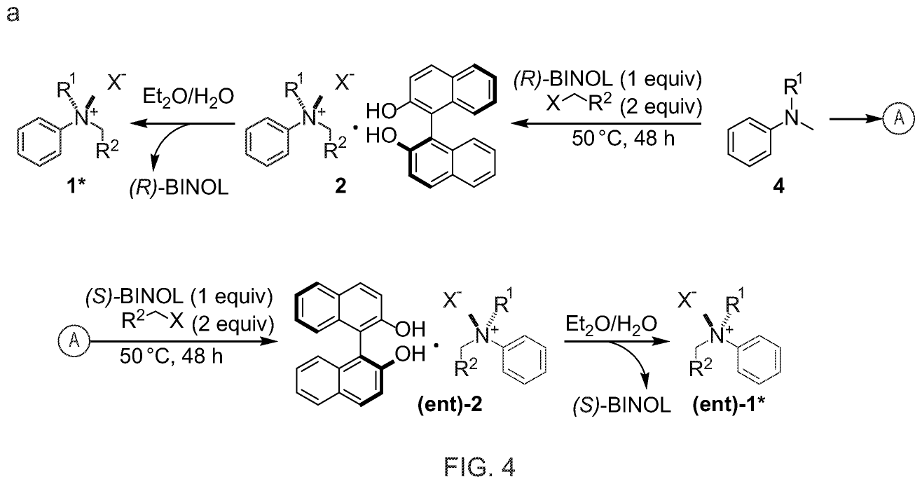

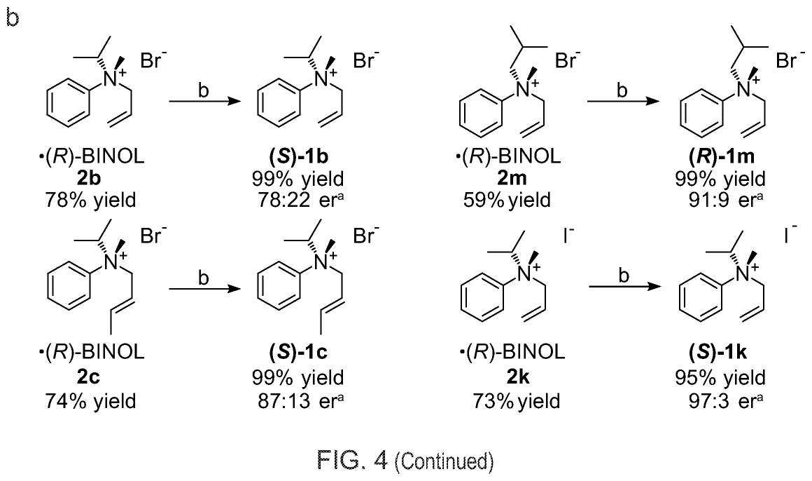

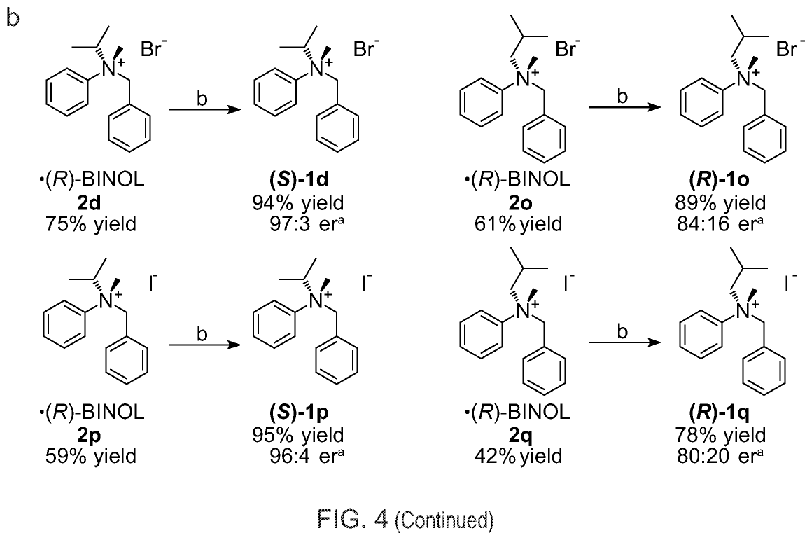

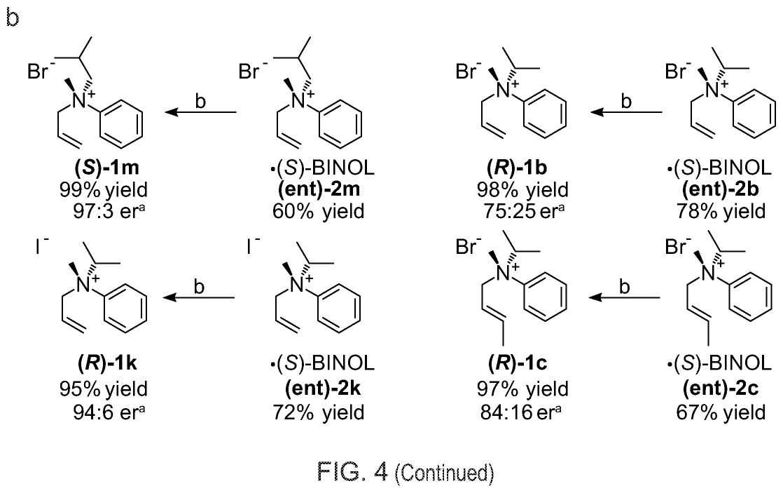

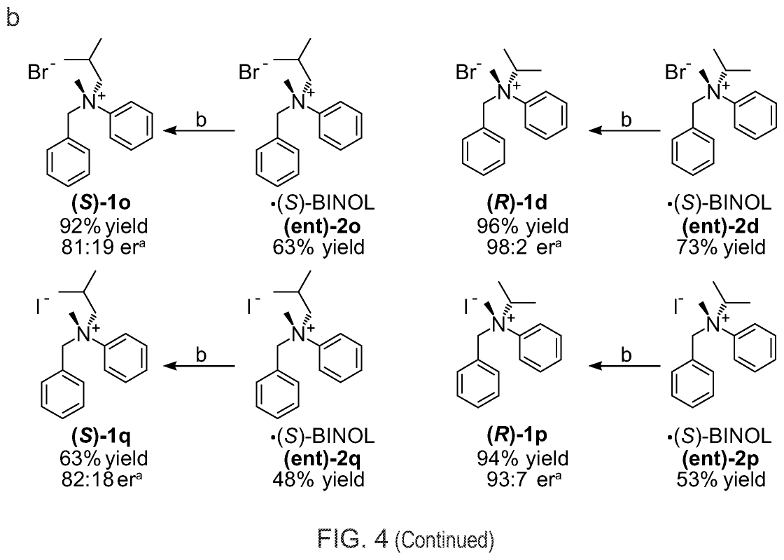

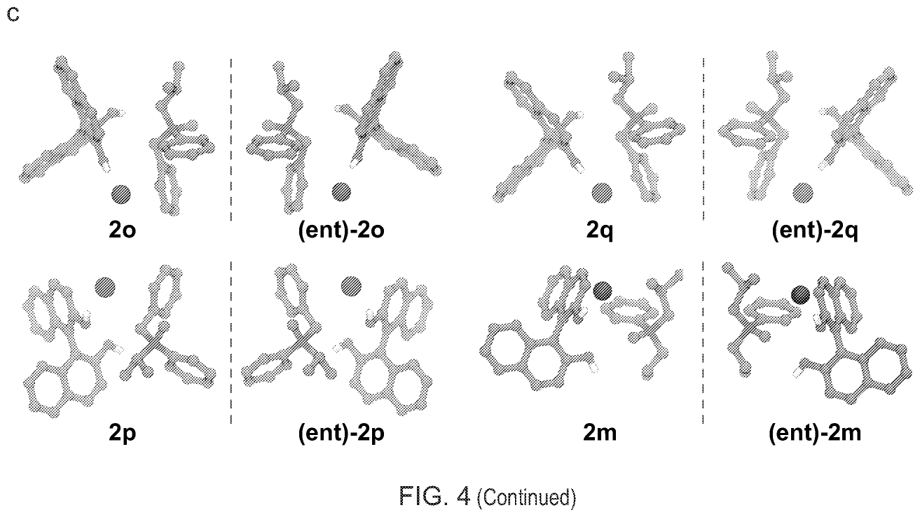

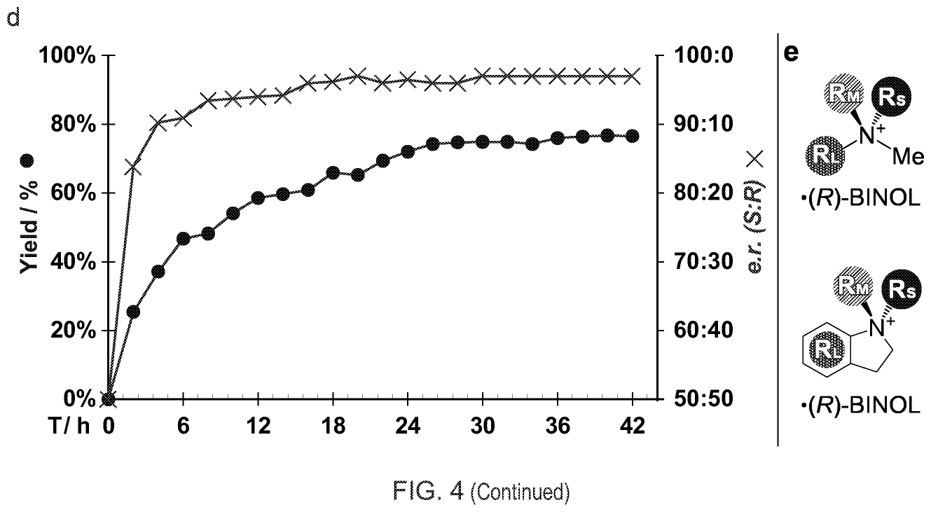

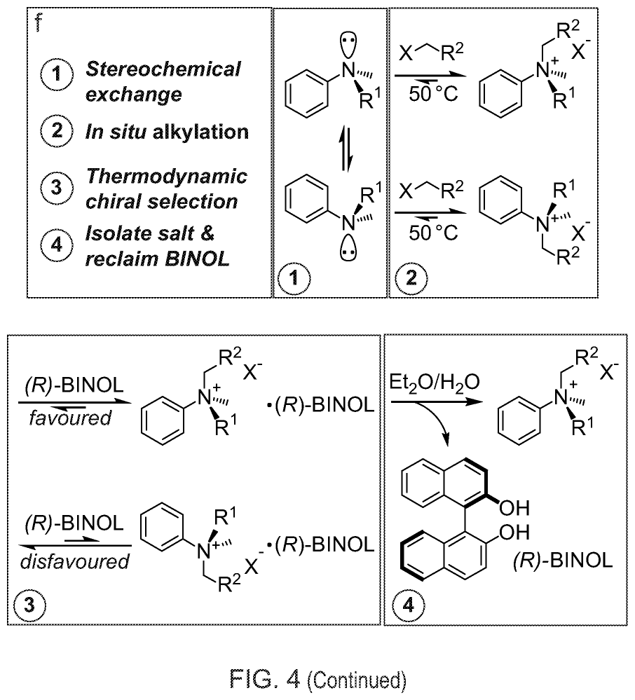

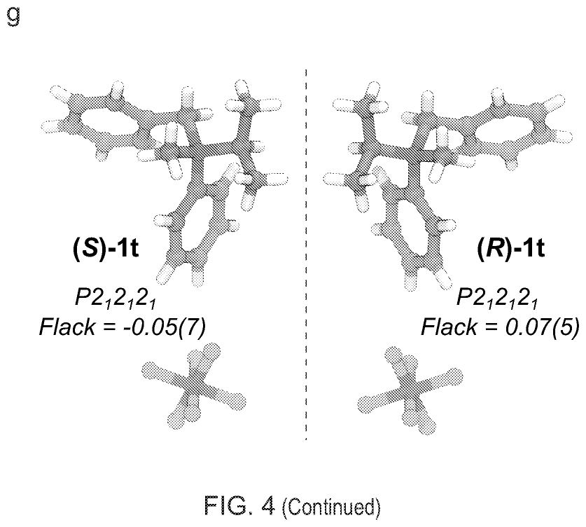

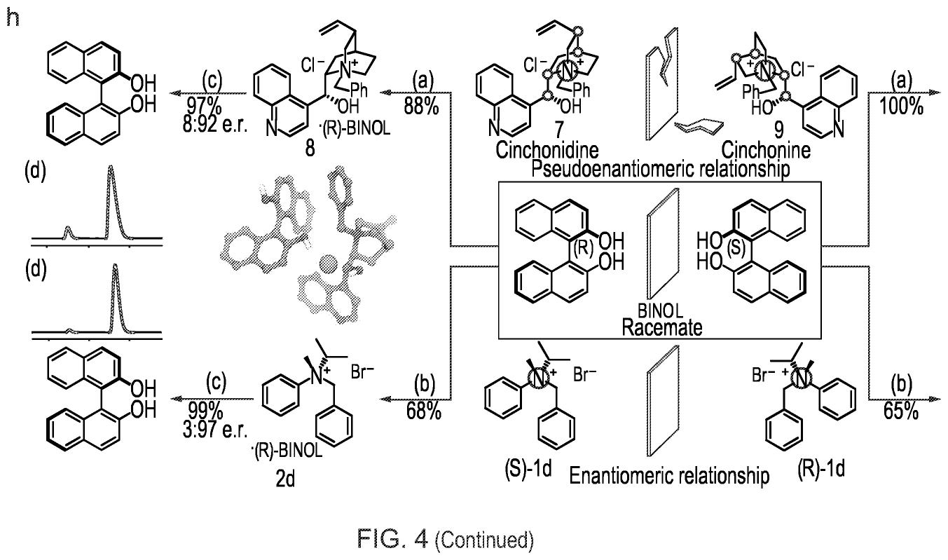

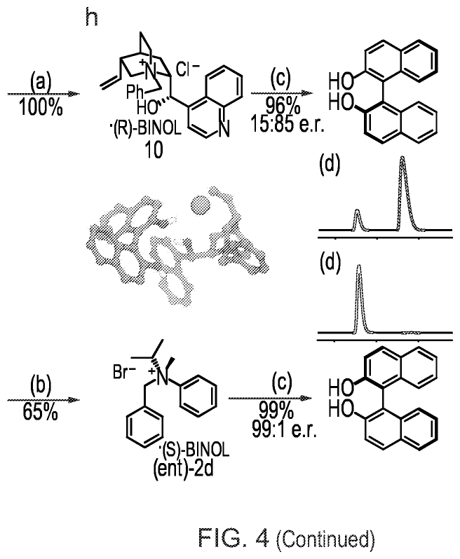

Combining these dynamic conditions with our recognition process allowed for the synthesis of enantioenriched chiral quaternary ammonium centres in a single pot. (FIG. 4a). Here, aniline 4, an alkylating agent (2 equiv.) and (R)-BINOL (1 equiv.) were combined under concentrated conditions (0.6-2.5 M) and stirred at 50° C. for 48 hours, generating the ternary complexes 2 as white solids. Both bromide (FIG. 4b. 2b-d, 2m-o) and iodide (2k, 2p-q) counter-ions mediated this process with a range of allylated (2b, 2k, 2m), crotylated (2c) and benzylated (2d, 2o, 2p, & 2q) chiral quaternary ammonium salts generated directly from the precursor anilines. The absolute configuration of each chiral quaternary ammonium cation was confirmed crystallographically (FIG. 4. c. 2m, 2o-2q, (ent)-2m, (ent)-2o-q).

Enantiomeric ratio (er) was determined by performing a counterion exchange of the isolated quaternary ammonium halide salt with the chiral shift reagent (R,Λ)-BINPHAT. The resulting diastereomeric salt was analysed by ¹H NMR spectroscopy.

Isolation of the enantioenriched quaternary ammonium salts was conducted by simple extraction with diethyl ether and water, which delivered excellent yields of the quaternary ammonium salts (1b-d, 1k, 1m, 1o-p; 63-99% yield) and reclaimed BINOL (62-98%).

Employing (S)-BINOL allowed access to the alternate enantiomers in comparable yields and selectivities (FIG. 4b. (ent)-1b-d, (ent)-1k, (ent)-1m, (ent)-1o-p).

Control reactions were carried out, with the results shown in Table 2, below. The control reactions highlighted variables of the process. Balancing temperature, concentration and equivalences of alkylating agent allowed for the ammonium stereocentre to be in dynamic exchange while allowing compatible recognition to occur. Analysis of the solution-phase component of these reactions showed that the uncomplexed ammonium remaining in solution was also biased towards the (S)-enantiomer (11% yield, 65:35 er (S:R). Demonstrating that both the solid and solution-phase ammonium cations have the same sense of enrichment confirms the enantioselective synthesis of an ammonium stereocentre.

TABLE 2
Control reactions assessing variables of the process.

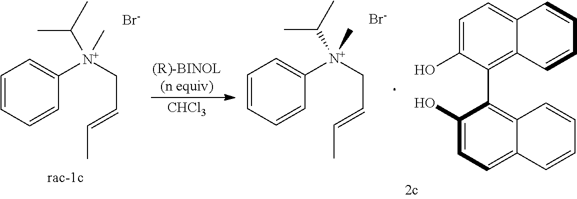

| Conditions | Time/h | Temp./° C. | Conc./M | Yield/% | dr |
|---|---|---|---|---|---|
| (R)-BINOL (1 equiv). TBAI (20 mol %) | 24 | 20 | 0.4 | 13 | — |
| (R)-BINOL (4 equiv), TBAI (20 mol %) | 48 | 20 | 0.4 | 77 | 48:52 |
| (R)-BINOL (1 equiv), allyl bromide (1 equiv) | 48 | 50 | 2 | 60 | 65:35 |
| (R)-BINOL (1 equiv), allyl bromide (1 equiv), | 120 | 50 | 1.5 | 45 | 76:34 |
| H₂O (5 equiv) | | | | | |
| (R)-BINOL (1 equiv), allyl bromide (1 equiv) | 48 | 50 | 0.6 | 74 | 82:18 |

Analysis of the progression of the reaction over time gave evidence of a self-correction process (FIG. 4d). In the time course analysis of the preparation of 2d, the yield of 2d rapidly increased over the initial 12 hours before slowly climbing to 75% after 26 hours. A similar increase was observed for the level of enantioenrichment over time, progressing from an initial value (T=2 hours, er=84:16) to a final equilibrium value (97:3 er) after approximately 16 hours. Such results are in stark contrast to kinetic resolution based processes, where a decrease in selectivity as the reaction proceeds is to be expected (see J. M. Keith, J. F. Larrow and E. N. Jacobsen, Adv. Synth. Catal. 343, 5-26 (2000)). This “error checking” feature offers further evidence of a thermodynamically driven process.

Without being bound by theory, a model for the selectivity observed is suggested in correlation with the results obtained, based on steric considerations. When the largest group is co-planar with the smallest group, either through free rotation or locked through the presence of a ring, the next largest group will occupy the proximal position when (R)-BINOL is employed (FIG. 4e).

Taking the above observations into account, and without being bound by theory, the inventors propose that the method of the invention occurs as outlined in FIG. 4f. The conformationally labile chiral tertiary amine can undergo a reversible, non-selective nucleophilic substitution, forming an equilibrium mixture of racemic quaternary ammonium cations and the initial tertiary amine. BINOL complexation to preferred quaternary ammonium salt enantiomers results in selection of the preferred enantiomer from solution, forming an enantioenriched ternary complex. Formation of the mismatched ternary complex also occurs, leading to initially moderate enantiomeric enrichment. However, given the relative instability of the mismatched structure, this complex preferentially dissolves, liberating the quaternary ammonium salt from the solid phase. The solution phase quaternary ammonium salt can revert back to the chiral tertiary amine, and undergo nucleophilic substitution again, possibly forming the preferred quaternary ammonium salt enantiomer, and re-complex BINOL in the preferred form leading to increased levels of enantioenrichment as the reaction progresses (see K. M. J. Brands and A. J. Davies, Chem. Rev., 106, 2711-2733 (2006)).

For unambiguous proof of the stereochemical stability of the chiral quaternary ammonium salt in the absence of BINOL, counter-ion exchange of 1d affording the crystalline hexafluorophosphate salt it was conducted for each enantiomer and examined crystallographically (FIG. 4g). Both enantiomeric salts crystallised within the chiral space group P212121 with configurations consistent with those observed in the respective ternary complexes. Analysis of the Flack parameters (H. D. Flack, Acta Cryst. A39, 876-881 (1983)) of the R (Flack=−0.05[7]) and S (Flack=0.07[5]) forms confirmed high levels of enantiomeric enrichment of each crystal. The configurational stabilities of these hexafluorophosphate salts were further proven when exposed to conditions previously shown to racemise the quaternary ammonium stereocentre in the presence of a bromide counterion. Heating a solution of it in acetonitrile at 50° C. for 24 hours led to no significant loss of optical activity of the sample. These results demonstrate that the once dynamic behaviour of this stereocentre can be frozen with preservation of chiral information at the nitrogen centre.

To demonstrate the significance of nitrogen stereocentres, the principal application of Cinchona alkaloids was investigated, namely their effectiveness in enantioselective supramolecular recognition. Such recognition is key to their catalytic capability and proficiency as resolution agents. The pseudoenantiomeric relationship between cinchonine and cinchonidine has been used to achieve enantioselective transformations with opposing senses of induction (see Genov et al., Science, 2020, 367, 1246-1251). Supramolecular recognition of BINOL with both pseudoenantiomeric forms of alkylated Cinchona alkaloids (see T. Toda and K. Tanaka, 1994, supra) provides ternary complexes enriched in (R)-BINOL (see FIG. 4h 8 and 10), which, once liberated, were observed in high enantiomeric enrichment (for 8, 92:8 e.r., R:S; for 10, 85:15 e.r., R:S). In contrast, when pure enantiomeric forms of ammonium stereocentres of the invention were used, the supramolecular recognition of BINOL was achieved, with (R)-1d forming a complex with (S)-BINOL (FIG. 4h for 2d, >1:99 e.r.) and its enantiomer (S)-1d providing (R)-BINOL (FIG. 4h for (ent)-2d, 97:3 e.r.) in excellent selectivity, surpassing the Cinchona-based counterparts. Given that both cinchonine and cinchonidine bear the same configuration at nitrogen, these results are consistent with stereogenic nitrogen providing a fundamental role in the supramolecular recognition observed in the Cinchona alkaloids. Accordingly, the invention allows for the interrogation of the role of ammonium stereocentres in all disciplines where tetra-alkylated ammonium cations are used.

### Enantioselective Recognition and Ternary Complex Characterisation Using (R)-BINOL

### (S)—N-allyl-N-ethyl-N-methylbenzenaminium bromide.(R)-1,1′-bi-2-naphthol (2a)

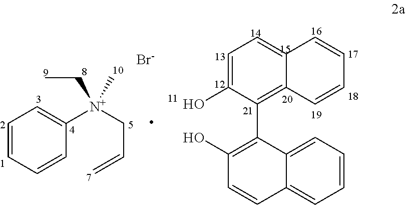 [SMILES: C=CC[N@+](C)(CC)c1ccccc1 ; [Br-] ; Oc1ccc2ccccc2c1-c1c(O)ccc2ccccc12]

Racemic N-allyl-N-ethyl-N-methylbenzenaminium bromide (0.254 g, 1.0 mmol) was dissolved into CHCl₃ (1.67 mL, 0.6 M) and deionised H₂O (90 μL, 5 equiv). Solid (R)-BINOL (0.143 g, 0.5 mmol) was added to the solution. After 5 mins, complexation was observed. The solution was stirred at room temperature for 18 h. The resulting precipitate was isolated by vacuum filtration to give 2a as a white solid (0.247 g, 91% yield).

¹H NMR (599 MHz, CD₃OD): δ 7.87 (d, J=8.9 Hz, 2H, H14), 7.83 (dd, J=8.2, 1.3 Hz, 2H, H16), 7.77-7.72 (m, 2H, H3), 7.68-7.62 (m, 2H, H2), 7.62-7.56 (m, 1H, H1), 7.30 (d, J=8.9 Hz, 2H, H19), 7.24 (ddd, J=8.1, 6.6, 1.3 Hz, 2H, H17), 7.16 (ddd, J=8.3, 6.8, 1.4 Hz, 2H, H18), 7.02 (d, J=8.5 Hz, 2H, H13), 5.65-5.50 (m, 3H, H6+7), 4.59 (dd, J=13.2, 5.8 Hz, 1H, H5), 4.36 (dd, J=13.2, 7.1 Hz, 1H, H5), 4.13-4.04 (m, 1H, H8), 3.83 (tdd, J=14.3, 7.2, 1.7 Hz, 1H, H8), 3.47 (s, 3H, H10), 1.10 (t, J=7.2 Hz, 3H, H9).

¹³C NMR (151 MHz, CD₃OD): δ 154.19 (C12), 142.89 (C4), 135.82 (C20), 131.75 (C2), 131.63 (C1), 130.56 (C14), 130.44 (C15), 129.19 (C7), 129.02 (C16), 127.12 (C18), 126.14 (C6), 125.81 (C13), 123.84 (C17), 122.97 (C3), 119.26 (C19), 116.20 (C21), 72.15 (C5), 64.63 (C8), 47.32 (C10), 8.70 (C9).

LRMS (ESI-TOF, EI+) m/z: 176.21 ([M]⁺, 100%), 162.26 (3), 136.12 (18).

LRMS (ESI-TOF, EI−) m/z: 285.26 ([M-H]⁻, 100%).

HRMS (ESI-TOF) m/z: [M]⁺ calculated for C₁₂H₁₈N⁺: 176.1439, found 176.1458. [M-H]⁻ calculated for C₂₀H₁₃O₂⁻: 285.0916, found 285.0906.

mp: 145-146° C., decomposed to red oil (EtOH).

[α]D=+14.00 (MeOH, c=0.5)

IR (cm⁻¹): 3125br, 2980m, 1622m, 1505m, 1430m, 1336m, 1272s, 954m, 817s, 754m.

XRD: A portion of the complex was crystallised in ethanol, resulting in clear colourless prisms. Crystal data: orthorhombic, space group P212121 (no. 19).

### (S)—N-allyl-N-isopropyl-N-methylbenzenaminium bromide.(R)-1,1′-bi-2-naphthol (2b)

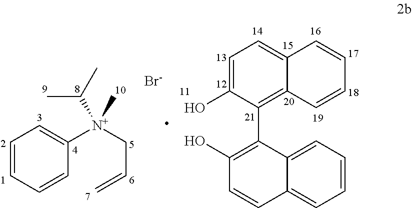 [SMILES: C=CC[N@+](C)(c1ccccc1)C(C)C ; [Br-] ; Oc1ccc2ccccc2c1-c1c(O)ccc2ccccc12]

Racemic N-allyl-N-isopropyl-N-methylbenzenaminium bromide (0.270 g, 1.0 mmol) was dissolved into CHCl₃ (1.67 mL, 0.6 M) and deionised H₂O (90 μL, 5 equiv).

Solid (R)-BINOL (0.143 g, 0.5 mmol) was added to the solution. After 15 mins, complexation was observed. The solution was stirred at room temperature for 18 h.

The resulting precipitate was isolated by vacuum filtration to give 2b as a white solid (0.195 g, 70% yield).

¹H NMR (599 MHz, CD₃OD): δ 7.87 (d, J=8.9 Hz, 2H, H14), 7.83 (dd, J=8.2, 1.3 Hz, 2H, H16), 7.73-7.69 (m, 2H, H3), 7.64-7.58 (m, 2H, H2), 7.58-7.54 (m, 1H, H1), 7.30 (d, J=8.9 Hz, 2H, H19), 7.24 (ddd, J=8.1, 6.6, 1.2 Hz, 2H, H17), 7.15 (ddd, J=8.2, 6.7, 1.3 Hz, 2H, H18), 7.02 (dd, J=8.5, 1.3 Hz, 2H, H13), 5.60-5.34 (m, 3H, H6+7), 4.68 (dd, J=13.3, 5.1 Hz, 1H, H5), 4.43-4.31 (m, 2H, H5+8), 3.30 (s, 3H, H10), 1.54 (d, J=6.4 Hz, 3H, H9), 1.03 (d, J=6.6 Hz, 3H, H9).

¹³C NMR (151 MHz, CD₃OD): δ 154.19 (C12), 143.82 (C4), 135.80 (C20), 131.62 (C2), 131.47 (C1), 130.55 (C14), 130.41 (C15), 129.03 (C16), 128.52 (C7), 127.13 (C18), 126.64 (C6), 125.81 (C13), 123.84 (C17), 123.31 (C3), 119.27 (C19), 116.22 (C21), 73.17 (C8), 68.59 (C5), 41.45 (C10), 17.29 (C9), 17.07 (C9).

LRMS (ESI-TOF, EI+) m/z: 190.27 ([M]⁺, 100%), 182.13 (1), 150.25 (2), 130.23 (2).

LRMS (ESI-TOF, EI−) m/z: 285.30 ([M-H]⁻, 100%).

HRMS (ESI-TOF) m/z: [M]⁺ calculated for C₁₃H₂₀N⁺: 190.1596, found 190.1597. [M-H]⁻ calculated for C₂₀H₁₃O₂⁺: 285.0916, found 285.0915.

mp: 150-152° C., decomposes to red oil (EtOH).

[α]D=+4.40 (MeOH, c=0.5)

IR (cm⁻¹): 3121br, 1623w, 1506w, 1273m, 950w, 813m, 688m.

XRD: Complex was crystallised in ethanol, resulting in clear colourless plates. Crystal data: tetragonal, space group P43 (no. 78).

### (S)-(E)-N-(but-2-en-1-yl)-N-isopropyl-N-methylbenzenaminium bromide.(R)-1,1′-bi-2-naphthol (2c)

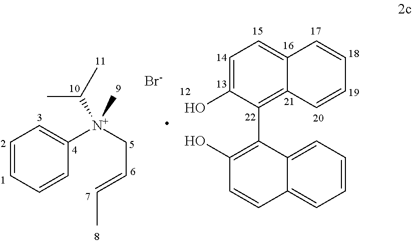 [SMILES: C/C=C/C[N@+](C)(c1ccccc1)C(C)C ; [Br-] ; Oc1ccc2ccccc2c1-c1c(O)ccc2ccccc12]

Racemic (E)-N-(but-2-en-1-yl)-N-isopropyl-N-methylbenzenaminium bromide (0.333 g, 1.17 mmol) was dissolved into CHCl₃ (1.67 mL, 0.6 M) and deionised H₂O (105 μL, 5 equiv). Solid (R)-BINOL (0.166 g, 0.59 mmol) was added to the solution. After 10 mins, complexation was observed. The solution was stirred at room temperature for 18 h. The resulting precipitate was isolated by vacuum filtration to give 2c as a white solid (0.255 g, 76% yield).

¹H NMR (599 MHz, CD₃OD): δ 7.87 (d, J=8.9 Hz, 2H, H15), 7.83 (dd, J=8.1, 1.2 Hz, 2H, H17), 7.72 (dd, J=7.9, 1.8 Hz, 2H, H3), 7.63 (dd, J=8.9, 7.1 Hz, 2H, H2), 7.58 (t, J=7.3 Hz, 1H, H1), 7.30 (d, J=8.9 Hz, 2H, H20), 7.24 (ddd, J=8.2, 6.7, 1.2 Hz, 2H, H18), 7.16 (ddd, J=8.2, 6.7, 1.3 Hz, 2H, H19), 7.02 (d, J=8.5 Hz, 2H, H14), 6.01 (dq, J=13.5, 6.6 Hz, 1H, H6), 5.03 (dddd, J=14.9, 8.2, 6.2, 1.8 Hz, 1H, H7), 4.60 (dd, J=13.1, 6.1 Hz, 1H, H5), 4.39-4.31 (m, 2H, H5+10), 3.29 (s, 3H, H9), 1.60 (dd, J=6.7, 1.4 Hz, 3H, H8), 1.55 (d, J=6.5 Hz, 3H, H11), 1.04 (d, J=6.6 Hz, 3H, H11).

¹³C NMR (151 MHz, CD₃OD): δ 154.20 (C13), 143.97 (C4), 141.86 (C6), 135.82 (C21), 131.58 (C2), 131.40 (C1), 130.55 (C15), 130.43 (C16), 129.02 (C17), 127.12 (C19), 125.81 (C14), 123.84 (C18), 123.35 (C3), 119.26 (C20), 119.21 (C7), 116.20 (C22), 72.56 (C10), 68.54 (C5), 41.07 (C9), 18.23 (C8), 17.36 (C11), 17.05 (C11).

LRMS (ESI-TOF, EI+) m/z: 204.67 ([M]⁺, 100%), 203.99 (82), 149.23 (2), 105.11 (1).

LRMS (ESI-TOF, EI−) m/z: 285.30 ([M-H]⁻, 100%).

HRMS (ESI-TOF) m/z: [M]⁺ calculated for C₁₄H₂₂N⁺: 204.1752, found 204.1757. [M-H]⁻ calculated for C₂₀H₁₃O₂⁻: 285.0916, found 285.0911.

mp: 150-153° C. (EtOH).

[α]D=+6.85 (MeOH, c=0.5)

IR (cm⁻¹): 3117br, 1622m, 1505m, 1272m, 814m, 687w.

XRD: A portion of the complex was crystallised in ethanol, resulting in clear colourless plates. Crystal data: tetragonal, space group P43 (no. 78).

### (S)—N-benzyl-N-isopropyl-N-methylbenzenaminium bromide.(R)-1,1′-bi-2-naphthol (2d)

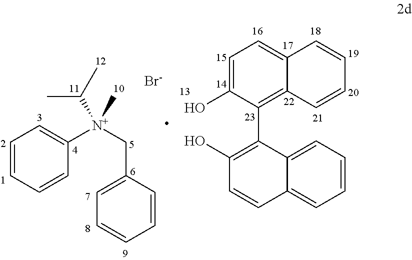 [SMILES: CC(C)[N@+](C)(Cc1ccccc1)c1ccccc1 ; [Br-] ; Oc1ccc2ccccc2c1-c1c(O)ccc2ccccc12]

Racemic N-benzyl-N-isopropyl-N-methylbenzenaminium bromide (0.160 g, 0.5 mmol) was dissolved into EtOH (0.75 mL, 1.0 M) and deionised H₂O (45 μL, 5 equiv). Solid (R)-BINOL (0.071 g, 0.25 mmol) was added to the solution. The solution was stirred at room temperature for 18 h. The resulting precipitate was isolated by vacuum filtration to give 2d as a white solid (0.097 g, 64% yield).

¹H NMR (700 MHz, CD₃OD): δ 7.87 (d, J=8.9 Hz, 2H, H16), 7.83 (d, J=8.1 Hz, 2H, H18), 7.69 (dd, J=7.2, 2.2 Hz, 2H, H3), 7.63-7.54 (m, 3H, H1+2), 7.34 (tt, J=7.5, 1.2 Hz, 1H, H9), 7.29 (d, J=8.9 Hz, 2H, H21), 7.24 (ddd, J=8.1, 6.7, 1.2 Hz, 2H, H19), 7.20 (dd, J=8.1 Hz, 2H, H8), 7.16 (ddd, J=8.2, 6.7, 1.3 Hz, 2H, H20), 7.02 (dd, J=8.5, 1.0 Hz, 2H, H15), 7.00 (dd, J=8.3, 0.8 Hz, 2H, H7), 5.12 (d, J=12.8 Hz, 1H, H5), 5.00 (d, J=12.8 Hz, 1H, H5), 4.71 (hept, J=6.5 Hz, 1H, H11), 3.27 (d, J=1.0 Hz, 3H, H10), 1.75 (d, J=6.4 Hz, 3H, H12), 1.08 (d, J=6.6 Hz, 3H, H12).

¹³C NMR (176 MHz, CD₃OD): δ 154.20 (C14), 143.63 (C4), 135.83 (C22), 133.90 (C7), 131.65 (C1), 131.49 (C2), 131.46 (C9), 130.56 (C16), 130.45 (C17), 129.63 (C8), 129.10 (C3), 129.02 (C18), 127.12 (C20), 125.81 (C15), 123.84 (C19), 123.77 (C6), 119.26 (C21), 116.19 (C23), 72.62 (C5), 70.55 (C11), 40.11 (C10), 17.59 (C12), 17.40 (C12).

HRMS (ESI-TOF) m/z: [M]⁺ calculated for C₁₇H₂₂N⁺: 240.1752, found 240.1738. [M-H]⁻ calculated for C₂₀H₁₃O₂: 285.0916, found 285.0921.

mp: 150-151° C. (EtOH).

[α]D=−45.57 (MeOH, c=0.5)

IR (cm⁻¹): 3139br, 2981m, 1622w, 1271s, 817s, 749s, 573w.

XRD: A portion of the ternary complex was crystallised in ethanol, resulting in clear colourless plates. Crystal data: orthorhombic, space group P212121 (no. 19).

### (R)-1-allyl-1-methylindolin-1-ium bromide.(R)-1,1′-bi-2-naphthol (2e)

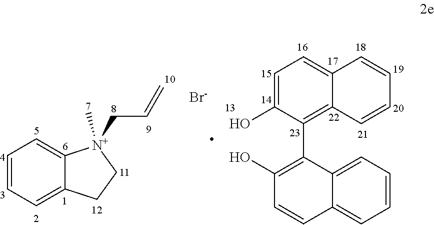 [SMILES: C=CC[N@@+]1(C)CCc2ccccc21 ; [Br-] ; Oc1ccc2ccccc2c1-c1c(O)ccc2ccccc12]

Racemic 1-allyl-1-methylindolin-1-ium bromide (0.254 g, 1.0 mmol) was dissolved into CHCl₃ (1.67 mL, 0.6 M) and deionised H₂O (90 μL, 5 equiv). Solid (R)-BINOL (0.143 g, 0.5 mmol) was added to the solution. After 5 mins, complexation was observed. The solution was stirred at room temperature for 18 h. The resulting precipitate was isolated by vacuum filtration to give 2e as a white solid (0.242 g, 89% yield).

¹H NMR (599 MHz, CD₃OD): δ 7.87 (d, J=8.9 Hz, 2H, H14), 7.83 (d, J=8.4 Hz, 2H, H16), 7.70 (d, J=8.1 Hz, 1H, H5), 7.60-7.51 (m, 3H, H2+3+4), 7.29 (d, J=8.9 Hz, 2H, H19), 7.24 (ddd, J=8.0, 6.7, 1.2 Hz, 2H, H17), 7.16 (ddd, J=8.2, 6.7, 1.4 Hz, 2H, H18), 7.02 (d, J=8.3 Hz, 2H, H13), 5.87 (ddt, J=17.2, 10.1, 7.3 Hz, 1H, H9), 5.68-5.61 (m, 2H, H10), 4.39 (dd, J=13.0, 7.7 Hz, 1H, H8), 4.35-4.28 (m, 2H, H11), 4.03 (dt, J=11.8, 7.9 Hz, 1H, H8), 3.49 (s, 3H, H7), 3.42-3.35 (m, 2H, H12).

¹³C NMR (151 MHz, CD₃OD): δ 154.19 (C14), 146.26 (t, J=6.2 Hz, C6), 135.82 (C22), 135.68 (t, J=2.4 Hz, C1), 132.46 (C4), 130.56 (C16), 130.44 (C2), 130.33 (C17), 129.58 (C3), 129.02 (C18), 127.97 (C10), 127.12 (C20), 126.50 (C9), 125.80 (C15), 123.84 (C9), 119.26 (C21), 119.15 (C5), 116.20 (C23), 69.71 (C8), 65.86 (C11), 53.40 (C7), 28.37 (C12).

LRMS (ESI-TOF, EI+) m/z: 174.57 ([M]⁺, 100%), 173.96 (73), 133.35 (1).

LRMS (ESI-TOF, EI−) m/z: 285.19 ([M-H]⁻, 100%).

HRMS (ESI-TOF) m/z: [M]⁺ calculated for C₁₂H₁₆N⁺: 174.1283, found 174.1277. [M-H]⁻ calculated for C₂₀H₁₃O₂⁻: 285.0916, found 285.0918.

mp: 173-174° C. (EtOH).

[α]D=+16.13 (MeOH, c=0.5).

IR (cm⁻¹): 3117br, 1621m, 1504m, 1431m, 1339m, 1273m, 955m, 817m, 755m, 686m.

XRD: The ternary complex was crystallised in ethanol, resulting in clear colourless plates. Crystal data: orthorhombic, space group P212121 (no. 19).

### (S)—N-allyl-N-cyclohexyl-N-methylbenzenaminium bromide.(R)-1,1′-bi-2-naphthol (2f)

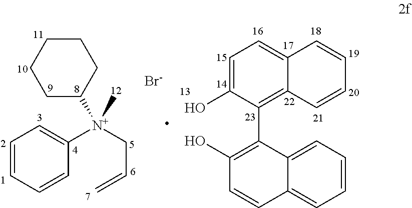 [SMILES: C=CC[N@+](C)(c1ccccc1)C1CCCCC1 ; [Br-] ; Oc1ccc2ccccc2c1-c1c(O)ccc2ccccc12]

Racemic N-allyl-N-cyclohexyl-N-methylbenzenaminium bromide (0.310 g, 1.0 mmol) was dissolved into CHCl₃ (1.0 mL, 1 M) and deionised H₂O (90 μL, 5 equiv). Solid (R)-BINOL (0.143 g, 0.5 mmol) was added to the solution. After 30 mins, complexation was observed. The solution was stirred at room temperature for 18 h. The resulting precipitate was isolated by vacuum filtration to give 2f as a white solid (0.158 g, 53% yield).

¹H NMR (599 MHz, CD₃OD): δ 7.87 (d, J=8.9 Hz, 2H, H16), 7.83 (d, J=8.2 Hz, 2H, H18), 7.68 (d, J=7.8 Hz, 2H, H3), 7.58 (t, J=8.0 Hz, 2H, H2), 7.52 (td, J=7.3, 1.1 Hz, 1H, H1), 7.32 (d, J=8.9 Hz, 2H, H21), 7.23 (ddd, J=8.1, 6.7, 1.3 Hz, 2H, H19), 7.14 (ddd, J=8.2, 6.7, 1.4 Hz, 2H, H20), 7.03 (d, J=8.5 Hz, 2H, H15), 5.60-5.45 (m, 1H, H6), 5.42-5.29 (m, 2H, H7), 4.65 (dd, J=13.2, 5.4 Hz, 1H, H5), 4.49-4.33 (m, 1H, H5), 3.99 (tt, J=11.7, 3.4 Hz, 1H, H8), 3.24 (s, 3H, H12), 2.38 (dq, J=10.8, 3.5 Hz, 1H, H9), 2.03-1.90 (m, 1H, H9), 1.71 (ddt, J=15.0, 5.7, 3.0 Hz, 1H, H9′), 1.66-1.58 (m, 1H, H9′), 1.58-1.41 (m, 2H, H10), 1.41-1.26 (m, 2H, H10′), 1.25-1.08 (m, 2H, H11).

¹³C NMR (151 MHz, CD₃OD): δ 154.17 (C14), 143.63 (C4), 135.77 (C22), 131.57 (C2), 131.33 (C1), 130.54 (C16), 130.37 (C17), 129.04 (C18), 128.54 (C7), 127.13 (C20), 126.51 (C6), 125.80 (C15), 123.85 (C19), 123.35 (C3), 119.28 (C21), 116.24 (C23), 80.43 (C8), 68.23 (C5), 42.76 (C12), 28.15 (C9), 27.90 (C9′), 26.51 (C10), 26.40 (C10′), 25.90 (C11).

LRMS (ESI-TOF, EI+) m/z: 230.28 ([M]⁺, 100%), 106.37 (9).

LRMS (ESI-TOF, EI−) m/z: 285.26 ([M-H]⁻, 100%).

HRMS (ESI-TOF) m/z: [M]⁺ Calculated for C₁₆H₂₄N⁺: 230.1909, found 230.1912. [M-H]⁻ Calculated for C₂₀H₁₃O₂⁻: 285.0916, found 285.0920.

mp: 158-160° C., decomposed to red oil (EtOH).

[α]D=+6.99 (MeOH, c=0.5)

IR (cm⁻¹): 3134br, 2940w, 1622m, 1430m, 1337m, 1272s, 958m, 818s, 755m, 694m.

XRD: A portion of the complex was crystallised in ethanol, resulting in clear colourless blocks. Crystal data: orthorhombic, space group P212121 (no. 19).

### (R)-1-(but-2-en-1-yl)-1-methylindolin-1-ium bromide.(R)-1,1′-bi-2-naphthol (2q)

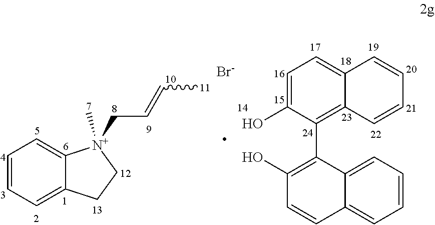

Racemic 1-(but-2-en-1-yl)-1-methylindolin-1-ium bromide (0.268 g, 1.0 mmol) was dissolved into CHCl₃ (1.67 mL, 0.6 M) and deionised H₂O (90 μL, 5 equiv). Solid (R)-BINOL (0.143 g, 0.5 mmol) was added to the solution. After 5 mins, complexation was observed. The solution was stirred at room temperature for 18 h. The resulting precipitate was isolated by vacuum filtration to give 2g as a white solid (0.247 g, 89% yield).

¹H NMR (599 MHz, DMSO-d₆): δ 9.19 (s, 2H, H14), 7.87-7.82 (m, 4H, H17+19), 7.81-7.78 (m, 1H, H5), 7.59-7.49 (m, 3H, H1+2+3), 7.32 (d, J=8.8 Hz, 2H, H22), 7.23 (ddd, J=8.1, 6.7, 1.2 Hz, 2H, H20), 7.16 (ddd, J=8.2, 6.7, 1.4 Hz, 2H, H21), 6.93 (dd, J=8.6, 1.2 Hz, 2H, H16), 6.12-5.93 (m, 1H, H9), 5.57-5.40 (m, 1H, H10), 4.52-4.16 (m, 3H, H8+12), 4.02 (ddt, J=42.2, 11.7, 7.9 Hz, 1H, H12), 3.43 (s, 3H, H7), 3.31-3.24 (m, 2H, H13), 1.70 (dd, J=6.6, 1.6 Hz, 3H, H11).

¹³C NMR (151 MHz, DMSO-d₆): δ 152.97 (C15), 145.04 (C6), 140.04 (C9), 137.24 (C1), 134.09 (C23), 130.70 (C3), 128.65 (C17), 128.60 (C4), 128.09 (C18), 127.82 (C19), 126.47 (C2), 125.80 (C21), 124.37 (C16), 122.23 (C20), 118.59 (C9), 118.51 (C22), 118.39 (C5), 115.36 (C24), 67.19 (C8), 63.63 (C12), 51.89 (C7), 27.06 (C13), 17.95 (C11).

LRMS (ESI-TOF, EI+) m/z: 188.21 ([M]⁺, 100%), 182.90 (1), 106.14 (23), 105.76 (3).

LRMS (ESI-TOF, EI−) m/z: 285.23 ([M-H]⁻, 100%) HRMS (ESI-TOF) m/z: [M]⁺ calculated for C₁₃H₁₃N⁺: 188.1439, found 188.1430. [M-H]⁻ calculated for C₂₀H₁₃O₂: 285.0916, found 285.0909.

mp: 179-180° C., decomposed to red oil (EtOH).

[α]D=+17.03 (MeOH, c=0.5).

IR (cm⁻¹): 3106br, 1622m, 1505m, 1430m, 1339m, 1271m, 977m, 817m, 753m, 573w.

XRD: A portion of the ternary complex was crystallised in ethanol, resulting in clear colourless plates. Crystal data: orthorhombic, space group P212121 (no. 19).

### (R)-1-(prop-2-yne)-1-methylindolin-1-ium bromide.(R)-1,1′-bi-2-naphthol (2h)

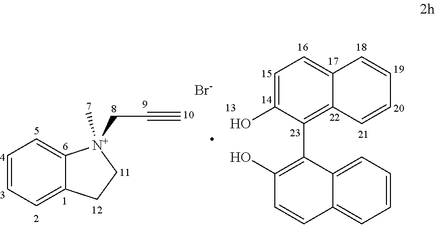 [SMILES: C#CC[N@@+]1(C)CCc2ccccc21 ; [Br-] ; Oc1ccc2ccccc2c1-c1c(O)ccc2ccccc12]

Racemic 1-(prop-2-yne)-1-methylindolin-1-ium bromide (0.200 g, 0.79 mmol) was dissolved into CHCl₃ (1.32 mL, 0.6 M) and deionised H₂O (71 μL, 5 equiv). Solid (R)-BINOL (0.113 g, 0.40 mmol) was added to the solution. After 10 mins, complexation was observed. The solution was stirred at room temperature for 18 h. The resulting precipitate was isolated by vacuum filtration to give 2h as a white solid (0.169 g, 79% yield).

¹H NMR (700 MHz, DMSO-d₆): δ 9.20 (s, 2H, H13), 7.88 (d, J=7.7 Hz, 1H, H5), 7.87-7.83 (m, 4H, H16+18), 7.60-7.53 (m, 3H, H2+3+4), 7.33 (d, J=8.9 Hz, 2H, H21), 7.23 (ddd, J=8.1, 6.7, 1.2 Hz, 2H, H19), 7.16 (ddd, J=8.2, 6.7, 1.4 Hz, 2H, H20), 6.93 (dd, J=8.4, 1.1 Hz, 2H, H15), 4.92 (qd, J=2.3 Hz, 2H, H8), 4.32 (dt, J=11.7, 7.3 Hz, 1H, H11), 4.20 (dt, J=11.7, 7.1 Hz, 1H, H11), 3.94 (t, J=2.5 Hz, 1H, H10), 3.54 (s, 3H, H7), 3.41 (t, J=7.3 Hz, 2H, H12).

¹³C NMR (176 MHz, DMSO-d₆): δ 152.96 (C14), 144.69 (C6), 134.51 (C17), 134.08 (C1), 131.07 (C3), 128.72 (C4), 128.58 (C16), 128.08 (C22), 127.81 (C18), 126.52 (C2), 125.78 (C20), 124.35 (C15), 122.21 (C19), 118.51 (C21), 118.31 (C5), 115.36 (C23), 82.45 (C10), 72.90 (C9), 65.39 (C11), 55.38 (C8), 52.55 (C7), 27.08 (C12).

HRMS (ESI-TOF) m/z: [M]⁺ calculated for C₁₂H₁₄N⁺: 172.1126, found 172.1126. [M-H]⁻ calculated for C₂₀H₁₃O₂: 285.0916, found 285.0875.

mp: 182-183° C. (EtOH).

[α]D=+21.72 (MeOH, c=0.5).

IR (cm⁻¹): 3130br, 2981m, 2136w, 1623m, 1271s, 818s.

XRD: The ternary complex was crystallised in ethanol, resulting in clear colourless blocks. Crystal data: orthorhombic, space group P212121 (no. 19).

### (R)-1-benzyl-1-methylindolin-1-ium bromide.(R)-1,1′-bi-2-naphthol (2i)

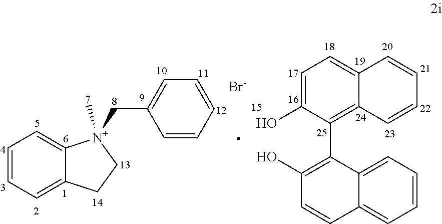 [SMILES: C[N@+]1(Cc2ccccc2)CCc2ccccc21 ; [Br-] ; Oc1ccc2ccccc2c1-c1c(O)ccc2ccccc12]

Racemic 1-benzyl-1-methylindolin-1-ium bromide (0.304 g, 1.0 mmol) was dissolved into CHCl₃ (1.67 mL, 0.6 M) and deionised H₂O (90 μL, 5 equiv). Solid (R)-BINOL (0.143 g, 0.5 mmol) was added to the solution. After 10 mins, complexation was observed. The solution was stirred at room temperature for 18 h. The resulting precipitate was isolated by vacuum filtration to give 2i as a white solid (0.239 g, 81% yield).

¹H NMR (599 MHz, CD₃OD): δ 7.87 (d, J=8.9 Hz, 2H, H18), 7.83 (d, J=8.2 Hz, 2H, H20), 7.67-7.63 (m, 1H, H5), 7.59-7.52 (m, 2H, H4+2), 7.51-7.45 (m, 1H, H12), 7.42-7.37 (m, 1H, H3), 7.35 (t, J=7.8 Hz, 2H, H21), 7.29 (d, J=8.9 Hz, 2H, H23), 7.24 (ddd, J=8.0, 6.7, 1.2 Hz, 2H, H22), 7.19-7.14 (m, 4H, H10+11), 7.06-6.99 (m, 2H, H17), 4.91 (d, J=12.7 Hz, 1H, H8), 4.80 (d, J=12.7 Hz, 1H, H8), 4.48 (ddd, J=11.7, 8.3, 3.0 Hz, 1H, H13), 3.99 (dt, J=11.3, 9.0 Hz, 1H, H13), 3.60 (s, 3H, H7), 3.16-3.07 (m, 1H, H14), 2.57 (dt, J=17.0, 8.9 Hz, 1H, H14).

¹³C NMR (151 MHz, CD₃OD): δ 154.19 (C16), 145.71 (t, J=5.8 Hz, C6), 136.56 (C1), 135.82 (C24), 133.60 (C11), 132.56 (C2), 131.98 (C12), 130.56 (C18), 130.44 (C19), 130.20 (C4), 130.03 (C21), 129.13 (C25), 129.02 (C9), 127.66 (C3), 127.12 (C10), 125.80 (C17), 123.84 (C22), 119.67 (C5), 119.26 (C23), 116.19 (C25), 71.98 (C8), 66.05 (C13), 53.63 (C7), 28.61 (C14).

HRMS (ESI-TOF) m/z: [M]⁺ calculated for C₁₅H₁₈N⁺: 224.1439, found 224.1421. [M-H]⁻ calculated for C₂₀H₁₃O₂⁻: 285.0916, found 285.0915.

mp: 189-190° C. (EtOH).

[α]D=+45.80 (MeOH, c=0.5).

IR (cm⁻¹): 3119br, 2982m, 1623w, 1329m, 1271s, 816s, 752s, 660w.

XRD: The ternary complex was crystallised in ethanol, resulting in clear colourless prisms. Crystal data: orthorhombic, space group P212121 (no. 19).

### (S)—N-benzyl-N-methyl-N-(isopropyl)prop-2-yn-1-aminium bromide.(R)-1,1′-bi-2-naphthol (2j)

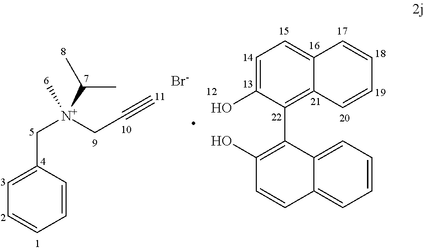 [SMILES: C#CC[N@@+](C)(Cc1ccccc1)C(C)C ; [Br-] ; Oc1ccc2ccccc2c1-c1c(O)ccc2ccccc12]

Racemic N-benzyl-N-methyl-N-(isopropyl)prop-2-yn-1-aminium bromide (0.282 g, 1.0 mmol) was dissolved into CHCl₃ (1.67 mL, 0.6 M) and deionised H₂O (90 μL, 5 equiv). Solid (R)-BINOL (0.143 g, 0.5 mmol) was added to the solution. Complexation was observed immediately upon addition. The solution was stirred at room temperature for 18 h. The resulting precipitate was isolated by vacuum filtration to give 2j as a white solid (0.224 g, 79% yield).

¹H NMR (400 MHz, DMSO-d₆): δ 9.22 (s, 2H, H12), 7.90-7.82 (m, 4H, H15+17), 7.74-7.62 (m, 2H, H3), 7.59-7.47 (m, 3H, H1+2), 7.34 (dd, J=8.9, 3.2 Hz, 2H, H20), 7.23 (td, J=8.0, 6.7, 1.8 Hz, 2H, H18), 7.17 (td, J=6.8, 3.5, 1.5 Hz, 2H, H19), 6.93 (d, J=8.5, 2.1 Hz, 2H, H14), 4.61 (d, J=13.0 Hz, 1H, H5), 4.47 (d, J=13.0 Hz, 1H, H5), 4.21-4.03 (m, 3H, H9+11), 3.93 (hept, J=6.6 Hz, 1H, H7), 2.89 (s, 3H, H6), 1.48 (d, J=6.5 Hz, 3H, H8), 1.44 (d, J=6.6 Hz, 3H, H8).

¹³C NMR (101 MHz, DMSO-d₆): δ 153.46 (C13), 134.57 (C21), 133.51 (C3), 130.91 (C1), 129.47 (C2), 129.09 (C16), 128.57 (C15), 128.33 (C17), 127.96 (C4), 126.30 (C19), 124.86 (C14), 122.73 (C18), 119.00 (C20), 115.85 (C22), 83.88 (C11), 73.03 (C10), 65.16 (C7), 61.91 (C5), 49.58 (C9), 43.97 (C6), 16.80 (C8), 16.78 (C8).

HRMS (ESI-TOF) m/z: [M]⁺ calculated for C₁₄H₂₀N⁺: 202.1596, found 202.1597. [M-H]⁻ calculated for C₂₀H₁₃O₂⁻: 285.0916, found 285.0941.

mp: 183° C. (EtOH).

[α]D=−3.21 (MeOH, c=0.5)

IR (cm⁻¹): 3139br, 2980m, 2137w, 1621m, 1329m, 1273s, 750s.

XRD: A portion of the ternary complex was crystallised in ethanol, resulting in clear colourless prisms. Crystal data: orthorhombic, space group P212121 (no. 19).

### (S)—N-allyl-N-isopropyl-N-methylbenzenaminium iodide.(R)-1,1′-bi-2-naphthol (2k)

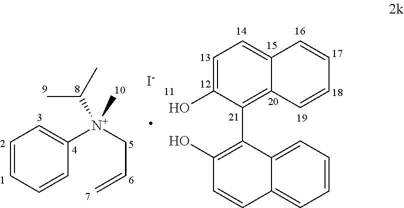 [SMILES: C=CC[N@+](C)(c1ccccc1)C(C)C ; [I-] ; Oc1ccc2ccccc2c1-c1c(O)ccc2ccccc12]

Racemic N-allyl-N-isopropyl-N-methylbenzenaminium iodide (0.302 g, 1.0 mmol) was dissolved into CHCl₃ (1.67 mL, 0.6 M) and deionised H₂O (90 μL, 5 equiv). Solid (R)-BINOL (0.143 g, 0.5 mmol) was added to the solution. After 30 seconds, complexation was observed. The solution was stirred at room temperature for 18 h. The resulting precipitate was isolated by vacuum filtration to give 2k as an off-white solid (0.162 g, 54% yield).

¹H NMR (700 MHz, CD₃OD): δ 7.87 (dd, J=8.9, 0.8 Hz, 2H, H14), 7.83 (d, J=8.3 Hz, 2H, H16), 7.76 (d, J=8.1 Hz, 2H, H3), 7.68-7.63 (m, 2H, H2), 7.59 (tt, J=7.4, 0.9 Hz, 1H, H1), 7.29 (d, J=8.9 Hz, 2H, H19), 7.24 (ddd, J=8.0, 6.7, 1.2 Hz, 2H, H17), 7.16 (ddd, J=8.2, 6.7, 1.3 Hz, 2H, H18), 7.03 (dd, J=8.5, 1.2 Hz, 2H, H13) 5.60-5.54 (m, 1H, H6), 5.45-5.39 (m, 2H, H7), 4.74-4.70 (m, 1H, H5), 4.46-4.43 (m, 1H, H5), 4.39 (hept, J=6.5 Hz, 1H, H8), 3.36 (s, 3H10), 1.59 (d, J=6.5 Hz, 3H, H9), 1.08 (d, J=6.6 Hz, 3H, H9).

¹³C NMR (176 MHz, CD₃OD): δ 154.19 (C12), 143.89 (C4), 135.83 (C20), 131.67 (C2), 131.55 (C1), 130.56 (C14), 130.45 (C15), 129.01 (C16), 128.53 (C7), 127.11 (C18), 126.70 (C6), 125.80 (C13), 123.83 (C17), 123.35 (C3), 119.25 (C19), 116.17 (C21), 73.23 (C8), 68.63 (C5), 41.52 (C10), 17.32 (C9), 17.08 (C9).

LRMS (ESI-TOF, EI+) m/z: 190.23 ([M]⁺, 100%), 165.19 (2).

LRMS (ESI-TOF, EI−) m/z: 285.08 ([M-H]⁻, 100%).

HRMS (ESI-TOF) m/z: [M]⁺ calculated for C₁₃H₂₀N⁺: 190.1596, found 190.1589. [M-H]⁻ calculated for C₂₀H₁₃O₂⁻: 285.0916, found 285.0900.

mp: 149-150° C. (EtOH).

[α]D=+3.51 (MeOH, c=0.5)

IR (cm⁻¹): 3220br, 2981w, 1621m, 1506m, 1434m, 1272s, 981m, 955m, 811s, 694m.

XRD: Complex crystallised in ethanol, resulting in clear colourless prisms. Crystal data: tetragonal, space group P43 (no. 78).

### (S)—N-isopropyl-N-propyl-N-methylbenzenaminium iodide.(R)-1,1′-bi-2-naphthol (2l)

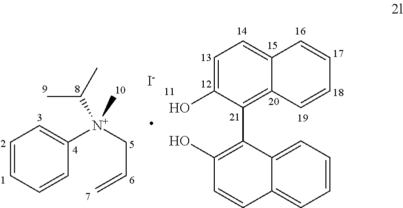 [SMILES: C=CC[N@+](C)(c1ccccc1)C(C)C ; [I-] ; Oc1ccc2ccccc2c1-c1c(O)ccc2ccccc12]

Racemic N-isopropyl-N-propyl-N-methylbenzenaminium iodide (0.319 g, 1.0 mmol) was dissolved into CHCl₃ (1.67 mL, 0.6 M) and deionised H₂O (90 μL, 5 equiv). Solid (R)-BINOL (0.143 g, 0.5 mmol) was added to the solution. After 30 seconds, complexation was observed. The solution was stirred at room temperature for 18 h. The resulting precipitate was isolated by vacuum filtration to give 2l as a white solid (0.249 g, 89% yield).

¹H NMR (700 MHz, CD₃OD): δ 7.87 (dd, J=8.9, 0.8 Hz, 2H, H14), 7.83 (d, J=8.2 Hz, 2H, H16), 7.78-7.72 (m, 2H, H3), 7.68-7.63 (m, 2H, H2), 7.62-7.57 (m, 1H, H1), 7.29 (d, J=8.9 Hz, 2H, H19), 7.24 (ddd, J=8.1, 6.7, 1.2 Hz, 2H, H17), 7.16 (ddd, J=8.2, 6.7, 1.3 Hz, 2H, H18), 7.02 (d, J=8.6 Hz, 2H, H13), 4.31 (hept, J=6.5 Hz, 1H, H8), 3.94 (td, J=12.6, 4.4 Hz, 1H, H5), 3.82 (ddd, J=12.8, 11.7, 5.0 Hz, 1H, H5), 3.38 (d, J=1.0 Hz, 3H, H10), 1.61 (tqd, J=12.4, 7.3, 4.9 Hz, 1H, H6), 1.55 (d, J=6.5 Hz, 3H, H9), 1.13-0.99 (m, 4H, H6+9), 0.92 (t, J=7.4 Hz, 3H, H7). 13C NMR (176 MHz, CD₃OD): δ 154.20 (C12), 144.01 (C4), 135.83 (C20), 131.72 (C2), 131.44 (C1), 130.56 (C14), 130.44 (C15), 129.02 (C16), 127.11 (C18), 125.81 (C13), 123.83 (C17), 123.10 (C3), 119.26 (C19), 116.18 (C21), 74.03 (C8), 67.30 (C5), 41.73 (C10), 17.99 (C9), 17.28 (C9), 17.05 (C6), 10.74 (C7).

HRMS (ESI-TOF) m/z: [M]⁺ calculated for C₁₃H₂₂N⁺: 192.1752, found 192.1735. [M-H]⁻ calculated for C₂₀H₁₃O₂⁻: 285.0916, found 285.0908.

mp: 189° C. (EtOH).

[α]D=+15.33 (MeOH, c=0.5)

IR (cm⁻¹): 3234br, 2975w, 1602w, 1434m, 1272s, 981m, 960m, 800s, 684m.

XRD: A portion of the ternary complex was crystallised in ethanol, resulting in clear colourless cubes. Crystal data: tetragonal, space group P43 (no. 78).

### Enantioselective Recognition and Ternary Complex Characterisation Using (S)-BINOL

### (R)—N-allyl-N-ethyl-N-methylbenzenaminium bromide.(S)-1,1′-bi-2-naphthol ((ent)-2a)

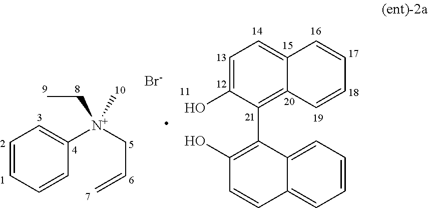 [SMILES: C=CC[N@+](C)(CC)c1ccccc1 ; [Br-] ; Oc1ccc2ccccc2c1-c1c(O)ccc2ccccc12]

Racemic N-allyl-N-ethyl-N-methylbenzenaminium bromide (0.256 g, 1.0 mmol) was dissolved into CHCl₃ (1.67 mL, 0.6 M) and deionised H₂O (90 μL, 5 equiv). Solid (S)-BINOL (0.143 g, 0.5 mmol) was added to the solution. After 5 mins, complexation was observed. The solution was stirred at room temperature for 18 h. The resulting precipitate was isolated by vacuum filtration to give (ent)-2a as a white solid (0.265 g, 97% yield).

¹H NMR (400 MHz, CD₃OD): spectrum identical to 2a 13C NMR (101 MHz, CD₃OD): spectrum identical to 2a HRMS (ESI-TOF) m/z: [M]⁺ calculated for C₁₂H₁₈N⁺: 176.1439, found 176.1432. [M-H]⁻ calculated for C₂₀H₁₃O₂⁻: 285.0916, found 285.0922.

mp: 144-145° C. decomposed to red oil (EtOH).

[α]D=−11.97 (MeOH, c=0.5).

XRD: A portion of the ternary complex was crystallised in ethanol, resulting in clear colourless blocks. Crystal data: orthorhombic, space group P212121 (no. 19).

### (R)—N-allyl-N-isopropyl-N-methylbenzenaminium bromide.(S)-1,1′-bi-2-naphthol ((ent)-2b)

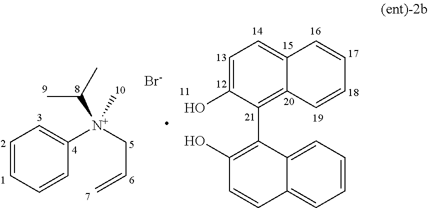 [SMILES: C=CC[N@@+](C)(c1ccccc1)C(C)C ; [Br-] ; Oc1ccc2ccccc2c1-c1c(O)ccc2ccccc12]

Racemic N-allyl-N-isopropyl-N-methylbenzenaminium bromide (0.270 g, 1.0 mmol) was dissolved into CHCl₃ (1.67 mL, 0.6 M) and deionised H₂O (90 μL, 5 equiv). Solid (S)-BINOL (0.143 g, 0.5 mmol) was added to the solution. After 15 mins, complexation was observed. The solution was stirred at room temperature for 18 h. The resulting precipitate was isolated by vacuum filtration to give (ent)-2b as a white solid (0.200 g, 72% yield).

¹H NMR (400 MHz, CD₃OD): spectrum identical to 2b

¹³C NMR (101 MHz, CD₃OD): spectrum identical to 2b

HRMS (ESI-TOF) m/z: [M]⁺ calculated for C₁₃H₂₀N⁺: 190.1596, found 190.1583. [M-H]⁻ calculated for C₂₀H₁₃O₂⁻: 285.0916, found 285.0924.

mp: 151-152° C., decomposes to red oil (EtOH).

[α]D=−3.28 (MeOH, c=0.5).

XRD: A portion of the ternary complex was crystallised in ethanol, resulting in clear colourless plates. Crystal data: tetragonal, space group P41 (no. 76).

### (R)-(E)-N-(but-2-en-1-yl)-N-isopropyl-N-methylbenzenaminium bromide.(S)-1,1′-bi-2-naphthol ((ent)-2c)

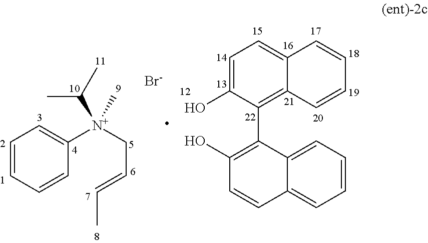 [SMILES: C/C=C/C[N@@+](C)(c1ccccc1)C(C)C ; [Br-] ; Oc1ccc2ccccc2c1-c1c(O)ccc2ccccc12]

Racemic (E)-N-(but-2-en-1-yl)-N-isopropyl-N-methylbenzenaminium bromide (0.284 g, 1.0 mmol) was dissolved into CHCl₃ (1.67 mL, 0.6 M) and deionised H₂O (90 μL, 5 equiv). Solid (S)-BINOL (0.143 g, 0.5 mmol) was added to the solution. After 10 mins, complexation was observed. The solution was stirred at room temperature for 18 h. The resulting precipitate was isolated by vacuum filtration to give (ent)-2c as a white solid (0.199 g, 70% yield).

¹H NMR (400 MHz, CD₃OD): spectrum identical to 2c

¹³C NMR (101 MHz, CD₃OD): spectrum identical to 2c

HRMS (ESI-TOF) m/z: [M]⁺ calculated for C₁₄H₂₂N⁺: 204.1752, found 204.1743. [M-H]⁻ calculated for C₂₀H₁₃O₂⁻: 285.0916, found 285.0899.

mp: 150-153° C. (EtOH).

[α]D=−4.78 (MeOH, c=0.5).

XRD: A portion of the ternary complex was crystallised in ethanol, resulting in clear colourless plates. Crystal data: tetragonal, space group P41 (no. 76).

### (R)—N-benzyl-N-isopropyl-N-methylbenzenaminium bromide.(S)-1,1′-bi-2-naphthol ((ent)-2d)

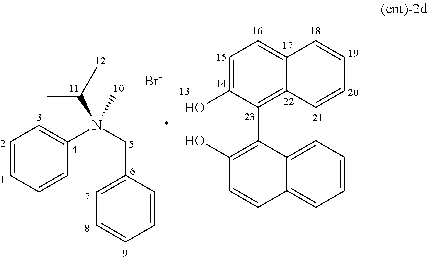 [SMILES: CC(C)[N@@+](C)(Cc1ccccc1)c1ccccc1 ; [Br-] ; Oc1ccc2ccccc2c1-c1c(O)ccc2ccccc12]

Racemic N-benzyl-N-isopropyl-N-methylbenzenaminium bromide (0.224 g, 0.7 mmol) was dissolved into EtOH (1.16 mL, 0.6 M) and deionised H₂O (63 μL, 5 equiv). Solid (S)-BINOL (0.100 g, 0.35 mmol) was added to the solution. The solution was stirred at room temperature for 18 h. The resulting precipitate was isolated by vacuum filtration to give (ent)-2d as a white solid (0.140 g, 66% yield).

¹H NMR (400 MHz, CD₃OD): spectrum identical to 2d

¹³C NMR (101 MHz, CD₃OD): spectrum identical to 2d

HRMS (ESI-TOF) m/z: [M]⁺ calculated for C₁₇H₂₂N⁺: 240.1752, found 240.1744. [M-H]⁻ calculated for C₂₀H₁₃O₂⁻: 285.0916, found 285.0913.

mp: 150-151° C. (EtOH).

[α]D=+47.79 (MeOH, c=0.5).

XRD: A portion of the ternary complex was crystallised in ethanol, resulting in clear colourless plates. Crystal data: orthorhombic, space group P212121 (no. 19).

### (S)-1-allyl-1-methylindolin-1-ium bromide.(S)-1,1′-bi-2-naphthol ((ent)-2e)

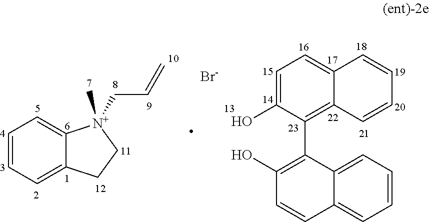 [SMILES: C=CC[N@+]1(C)CCc2ccccc21 ; [Br-] ; Oc1ccc2ccccc2c1-c1c(O)ccc2ccccc12]

Racemic 1-allyl-1-methylindolin-1-ium bromide (0.254 g, 1.0 mmol) was dissolved into CHCl₃ (1.67 mL, 0.6 M) and deionised H₂O (90 μL, 5 equiv). Solid (S)-BINOL (0.143 g, 0.5 mmol) was added to the solution. After 5 mins, complexation was observed. The solution was stirred at room temperature for 18 h. The resulting precipitate was isolated by vacuum filtration to give (ent)-2e as a white solid (0.275 g, 100% yield).

¹H NMR (400 MHz, CD₃OD): spectrum identical to 2e

¹³C NMR (101 MHz, CD₃OD): spectrum identical to 2e

HRMS (ESI-TOF) m/z: [M]⁺ calculated for C₁₂H₁₆N⁺: 174.1283, found 174.1281. [M-H]⁻ calculated for C₂₀H₁₃O₂⁻: 285.0916, found 285.0924.

mp: 176-178° C. (EtOH).

[α]D=−17.68 (MeOH, c=0.5).

XRD: A portion of the ternary complex was crystallised in ethanol, resulting in clear colourless blocks. Crystal data: orthorhombic, space group P212121 (no. 19).

### (R)—N-allyl-N-cyclohexyl-N-methylbenzenaminium bromide.(S)-1,1′-bi-2-naphthol ((ent)-2f)

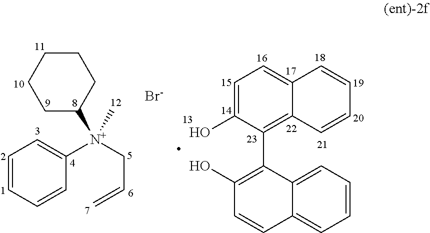 [SMILES: C=CC[N@@+](C)(c1ccccc1)C1CCCCC1 ; [Br-] ; Oc1ccc2ccccc2c1-c1c(O)ccc2ccccc12]

Racemic N-allyl-N-cyclohexyl-N-methylbenzenaminium bromide (0.310 g, 1.0 mmol) was dissolved into CHCl₃ (1.0 mL, 1 M) and deionised H₂O (90 μL, 5 equiv). Solid (S)-BINOL (0.143 g, 0.5 mmol) was added to the solution. The solution was stirred at room temperature for 18 h. The resulting precipitate was isolated by vacuum filtration to give (ent)-2f as a white solid (0.168 g, 56% yield).

¹H NMR (400 MHz, CD₃OD): spectrum identical to 2f

¹³C NMR (101 MHz, CD3OD): spectrum identical to 2f

HRMS (ESI-TOF) m/z: [M]⁺ Calculated for C₁₆H₂₄N⁺: 230.1909, found 230.1901. [M-H]⁻ Calculated for C₂₀H₁₃O₂⁻: 285.0916, found 285.0920.

mp: 152-154° C. (EtOH).

[α]D=−3.57 (MeOH, c=0.5).

XRD: A portion of the ternary complex was crystallised in ethanol, resulting in clear colourless blocks. Crystal data: orthorhombic, space group P212121 (no. 19).

### (S)-1-(but-2-en-1-yl)-1-methylindolin-1-ium bromide.(S)-1,1′-bi-2-naphthol ((ent)-2q)

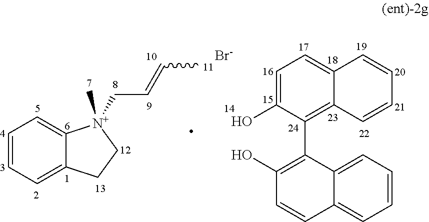

Racemic 1-(but-2-en-1-yl)-1-methylindolin-1-ium bromide (0.268 g, 1.0 mmol) was dissolved into CHCl₃ (1.67 mL, 0.6 M) and deionised H₂O (90 μL, 5 equiv). Solid (S)-BINOL (0.143 g, 0.5 mmol) was added to the solution. After 5 mins, complexation was observed. The solution was stirred at room temperature for 18 h. The resulting precipitate was isolated by vacuum filtration to give (ent)-2g as a white solid (0.216 g, 78% yield).

¹H NMR (400 MHz, CD₃OD): spectrum identical to 2g

¹³C NMR (101 MHz, CD₃OD): spectrum identical to 2g

HRMS (ESI-TOF) m/z: [M]⁺ calculated for C₁₃H₁₃N⁺: 188.1439, found 188.1435. [M-H]⁻ calculated for C₂₀H₁₃O₂: 285.0916, found 285.0913.

mp: 171-172° C. (EtOH).

[α]D=−24.90 (MeOH, c=0.5).

XRD: A portion of the ternary complex was crystallised in ethanol, resulting in clear colourless needles. Crystal data: orthorhombic, space group P212121 (no. 19).

### (S)-1-(prop-2-yne)-1-methylindolin-1-ium bromide.(S)-1,1′-bi-2-naphthol ((ent)-2h)

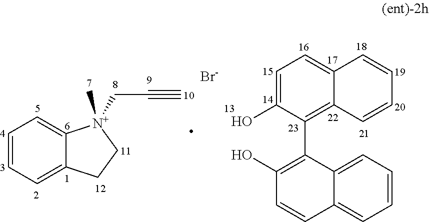 [SMILES: C#CC[N@+]1(C)CCc2ccccc21 ; [Br-] ; Oc1ccc2ccccc2c1-c1c(O)ccc2ccccc12]

Racemic 1-(prop-2-yne)-1-methylindolin-1-ium bromide (0.200 g, 0.79 mmol) was dissolved into CHCl₃ (1.32 mL, 0.6 M) and deionised H₂O (71 μL, 5 equiv). Solid (S)-BINOL (0.113 g, 0.5 mmol) was added to the solution. After 10 mins, complexation was observed. The solution was stirred at room temperature for 18 h. The resulting precipitate was isolated by vacuum filtration to give (ent)-2h as a white solid (0.185 g, 87% yield).

¹H NMR (400 MHz, DMSO-d₆): spectrum identical to 2h

¹³C NMR (101 MHz, DMSO-d₆): spectrum identical to 2h

HRMS (ESI-TOF) m/z: [M]⁺ calculated for C₁₂H₁₄N⁺: 172.1126, found 172.1123. [M-H]⁻ calculated for C₂₀H₁₃O₂⁻: 285.0916, found 285.0916.

mp: 179-180° C. (EtOH).

[α]D=−13.24 (MeOH, c=0.5).

XRD: A portion of the ternary complex was crystallised in ethanol, resulting in clear yellowish prisms. Crystal data: orthorhombic, space group P212121 (no. 19).

### (S)-1-benzyl-1-methylindolin-1-ium bromide.(S)-1,1′-bi-2-naphthol ((ent)-2i)

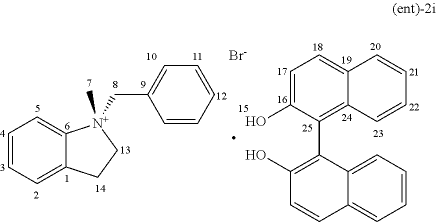 [SMILES: C[N@@+]1(Cc2ccccc2)CCc2ccccc21 ; [Br-] ; Oc1ccc2ccccc2c1-c1c(O)ccc2ccccc12]

Racemic ammonium salt 1-benzyl-1-methylindolin-1-ium bromide (0.304 g, 1.0 mmol) was dissolved into CHCl₃ (1.67 mL, 0.6 M) and deionised H₂O (90 μL, 5 equiv). Solid (S)-BINOL (0.143 g, 0.5 mmol) was added to the solution. After 5 mins, complexation was observed. The solution was stirred at room temperature for 18 h. The resulting precipitate was isolated by vacuum filtration to give (ent)-2i as a white solid (0.233 g, 79% yield).

¹H NMR (400 MHz, CD₃OD): spectrum identical to 2i

¹³C NMR (101 MHz, CD₃OD): spectrum identical to 2i

HRMS (ESI-TOF) m/z: [M]⁺ calculated for C₁₆H₁₈N⁺: 224.1439, found 224.1436. [M-H]⁻ calculated for C₂₀H₁₃O₂: 285.0916, found 285.0930.

mp: 189-190° C. (EtOH).

[α]D=−51.02 (MeOH, c=0.5).

XRD: A portion of the ternary complex was crystallised in ethanol, resulting in clear colourless prisms. Crystal data: orthorhombic, space group P212121 (no. 19).

### (R)—N-benzyl-N-methyl-N-(isopropyl)prop-2-yn-1-aminium bromide.(S)-1,1′-bi-2-naphthol ((ent)-2j)

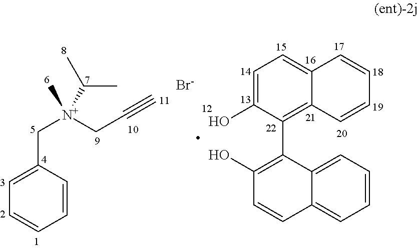 [SMILES: C#CC[N@+](C)(Cc1ccccc1)C(C)C ; [Br-] ; Oc1ccc2ccccc2c1-c1c(O)ccc2ccccc12]

Racemic N-benzyl-N-methyl-N-(isopropyl)prop-2-yn-1-aminium bromide (0.282 g, 1.0 mmol) was dissolved into CHCl₃ (1.67 mL, 0.6 M) and deionised H₂O (90 μL, 5 equiv). Solid (S)-BINOL (0.143 g, 0.5 mmol) was added to the solution. After 15 mins, complexation was observed. The solution was stirred at room temperature for 18 h. The resulting precipitate was isolated by vacuum filtration to give (ent)-2j as a white solid (0.253 g, 89% yield).

¹H NMR (400 MHz, DMSO-d₆): spectrum identical to 2j

¹³C NMR (101 MHz, DMSO-d₆): spectrum identical to 2j

HRMS (ESI-TOF) m/z: [M]⁺ calculated for C₁₄H₂₀N⁺: 202.1596, found 202.1585. [M-H]⁻ calculated for C₂₀H₁₃O₂⁻: 285.0916, found 285.0910.

mp: 184° C. (EtOH).

[α]D=+5.11 (MeOH, c=0.5).

XRD: A portion of the ternary complex was crystallised in ethanol, resulting in clear colourless plates. Crystal data: orthorhombic, space group P212121 (no. 19).

### (R)—N-allyl-N-isopropyl-N-methylbenzenaminium iodide.(S)-1,1′-bi-2-naphthol ((ent)-2k)

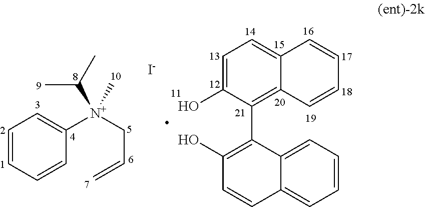 [SMILES: C=CC[N@@+](C)(c1ccccc1)C(C)C ; [I-] ; Oc1ccc2ccccc2c1-c1c(O)ccc2ccccc12]

Racemic N-allyl-N-isopropyl-N-methylbenzenaminium iodide (0.302 g, 1.0 mmol) was dissolved into CHCl₃ (1.67 mL, 0.6 M) and deionised H₂O (90 μL, 5 equiv). Solid (S)-BINOL (0.143 g, 0.5 mmol) was added to the solution. After 30 seconds, complexation was observed. The solution was stirred at room temperature for 18 h. The resulting precipitate was isolated by vacuum filtration to give (ent)-2k as an off white solid (0.164 g, 56% yield).

¹H NMR (400 MHz, CD₃OD): spectrum identical to 2k

¹³C NMR (101 MHz, CD₃OD): spectrum identical to 2k

HRMS (ESI-TOF) m/z: [M]⁺ calculated for C₁₃H₂₀N⁺: 190.1596, found 190.1599. [M-H]⁻ calculated for C₂₀H₁₃O₂⁻: 285.0916, found 285.0927.

mp: 146° C. (EtOH)

[α]D=−3.26 (MeOH, c=0.5).

XRD: A portion of the ternary complex was crystallised in ethanol, resulting in clear colourless plates. Crystal data: tetragonal, space group P41 (no. 76).

### (R)—N-isopropyl-N-propyl-N-methylbenzenaminium iodide.(S)-1,1′-bi-2-naphthol ((ent)-2l)

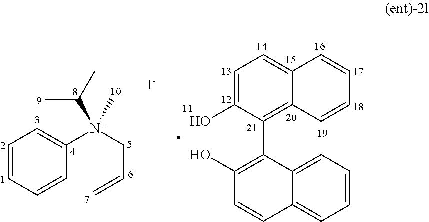 [SMILES: C=CC[N@@+](C)(c1ccccc1)C(C)C ; [I-] ; Oc1ccc2ccccc2c1-c1c(O)ccc2ccccc12]

Racemic N-isopropyl-N-propyl-N-methylbenzenaminium iodide (0.319 g, 1.0 mmol) was dissolved into CHCl₃ (1.67 mL, 0.6 M) and deionised H₂O (90 μL, 5 equiv). Solid (S)-BINOL (0.143 g, 0.5 mmol) was added to the solution. After 30 seconds, complexation was observed. The solution was stirred at room temperature for 18 h. The resulting precipitate was isolated by vacuum filtration to give (ent)-11 as a white solid (0.226 g, 75% yield).

¹H NMR (400 MHz, CD₃OD): spectrum identical to 2l

¹³C NMR (101 MHz, CD₃OD): spectrum identical to 2l

HRMS (ESI-TOF) m/z: [M]⁺ calculated for C₁₃H₂₂N⁺: 192.1752, found 192.1728. [M-H]⁻ calculated for C₂₀H₁₃O₂⁻: 285.0916, found 285.0929.

mp: 188° C. (EtOH).

[α]D=−16.51 (MeOH, c=0.5).

XRD: A portion of the ternary complex was crystallised in ethanol, resulting in clear pinkish plates. Crystal data: tetragonal, space group P41 (no. 76).

### Synthesis of Mis-Matched Ternary Complex

### (S)—N-allyl-N-isopropyl-N-methylbenzenaminium bromide.(S)-1,1′-bi-2-naphthol ((S)-1b. (S)-BINOL)

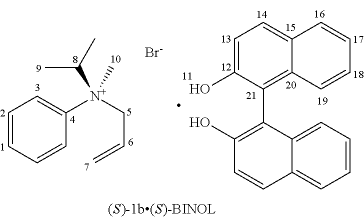 [SMILES: C=CC[N@@+](C)(c1ccccc1)C(C)C ; [Br-] ; Oc1ccc2ccccc2c1-c1c(O)ccc2ccccc12]

Enriched complex 2b was recrystallised to higher enantiomeric purity (90:10 er) in EtOH, before decomplexation in Et₂O and deionised H₂O. Collecting the aqueous phases and concentration under reduced pressure recovered enriched salt (S)-1b. (S)-1b (0.053 g, 0.19 mmol) was dissolved into CHCl₃ (0.4 mL, 0.6 M). Solid (S)-BINOL (0.056 g, 0.19 mmol) was added to the solution with stirring. After 15 mins, complexation was observed, and the solution was left to stir overnight. After vacuum filtration, a white precipitate of (S)-1b.(S)-BINOL was isolated (0.078 g, 74% yield).

¹H NMR (400 MHz, CD₃OD): identical to compound 2b

¹³C NMR (101 MHz, CD₃OD): identical to compound 2b

HRMS (ESI-TOF) m/z: [M]⁺ calculated for C₁₃H₂₀N⁺: 190.1590, found 190.1577. [M-H]⁻ calculated for C₂₀H₁₃O₂⁻: 285.0916, found 285.0938.

mp: 137-139° C.

[α]D=24.53 (MeOH, c=0.5).

XRD: A portion of the ternary complex was crystallised in ethanol, resulting in clear colourless prisms. Crystal data: orthorhombic, space group P212121 (no. 19).

### Synthesis of Enantio-Enriched Quaternary Ammonium Salts Using (R)-BINOL

### (S)—N-allyl-N-isopropyl-N-methylbenzenaminium bromide.(R)-1,1′-bi-2-naphthol (2b)

A solution of N-isopropyl-N-methylaniline (0.298 g, 2.0 mmol) was prepared in CHCl₃ (3.2 mL, 0.6 M). Allyl bromide (0.38 mL, 4.0 mmol) was added with stirring and allowed to react for 10 min. Solid (R)-BINOL (0.572 g, 2.0 mmol) was added to the solution and the reaction vessel sealed and heated to 50° C. After 48 h of heating, the mixture was filtered to isolate 2b as a white solid (0.876 g, 79% yield).

Characterisation data is as given above for 2b.

mp: 151-152° C. decomposed to red oil (EtOH).

[α]D=+4.75 (MeOH, c=0.5).

### (S)-(E)-N-(but-2-en-1-yl)-N-isopropyl-N-methylbenzenaminium bromide.(R)-1,1′-bi-2-naphthol (2c)

A solution of N-isopropyl-N-methylaniline (0.149 g, 1.0 mmol) was prepared in CHCl₃ (1.67 mL, 0.6 M). Crotyl bromide (0.20 mL, 2.0 mmol) is added with stirring and allowed to react for 10 min. Solid (R)-BINOL (0.286 g, 1.0 mmol) was added to the solution and the reaction vessel sealed and heated to 50° C. After 48 h of heating, the mixture was filtered to isolate 2c as a white solid (0.381 g, 67% yield).

Characterisation data is as given above for 2c.

mp: 150-151° C. (EtOH).

[α]D=+6.89 (MeOH, c=0.5).

### (S)—N-benzyl-N-isopropyl-N-methylbenzenaminium bromide.(R)-1,1′-bi-2-naphthol (2d)

A solution of N-isopropyl-N-methylaniline (0.149 g, 1.0 mmol) was prepared in MeCN (1.67 mL, 0.6 M). Benzyl bromide (0.24 mL, 2.0 mmol) was added with stirring and allowed to react for 10 min. Solid (R)-BINOL (0.286 g, 1.0 mmol) was added to the solution and the reaction vessel sealed and heated to 50° C. After 48 h of heating, the mixture was filtered to isolate 2d as a white solid (0.473 g, 78% yield).

Characterisation data is as given above for 2d.

mp: 152-153° C. (EtOH).

[α]D=−51.28 (MeOH, c=0.5).

### (S)—N-allyl-N-isopropyl-N-methylbenzenaminium iodide.(R)-1,1′-bi-2-naphthol (2k)

A solution of N-isopropyl-N-methylaniline (0.149 g, 1.0 mmol) was prepared in CHCl₃ (1.67 mL, 0.6 M). Allyl iodide (0.18 mL, 2.0 mmol) was added with stirring and allowed to react for 10 min. Solid (R)-BINOL (0.286 g, 1.0 mmol) was added to the solution and the reaction vessel sealed and heated to 50° C. After 48 h of heating, the mixture was filtered to isolate 2k as a pale yellow solid (0.440 g, 73% yield).

Characterisation data is as given above for 2k.

mp: 148-150° C. (EtOH).

[α]D=+2.33 (MeOH, c=0.5).

### (R)—N-allyl-N-(2-methylpropyl)-N-methylbenzenaminium bromide.(R)-1,1′-bi-2-naphthol (2m)

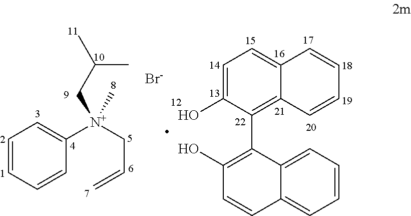 [SMILES: C=CC[N@+](C)(CC(C)C)c1ccccc1 ; [Br-] ; Oc1ccc2ccccc2c1-c1c(O)ccc2ccccc12]

A solution of N-isobutyl-N-methylaniline (0.163 g, 1.0 mmol) was prepared in CHCl₃ (1.67 mL, 0.6 M). Allyl bromide (0.19 mL, 2.0 mmol) was added with stirring and allowed to react for 10 min. Solid (R)-BINOL (0.286 g, 1.0 mmol) was added to the solution and the reaction vessel sealed and heated to 50° C. After 48 h of heating, the mixture was filtered to isolate 2m as a white solid (0.337 g, 59% yield).

¹H NMR (700 MHz, CD₃OD): δ 7.87 (dd, J=8.9, 0.8 Hz, 2H, H15), 7.83 (d, J=8.2 Hz, 2H, H17), 7.82-7.79 (m, 2H, H3), 7.67-7.62 (m, 2H, H2), 7.61-7.57 (m, 1H, H1), 7.30 (d, J=8.9 Hz, 2H, H20), 7.24 (ddd, J=8.1, 6.7, 1.2 Hz, 2H, H18), 7.16 (ddd, J=8.2, 6.7, 1.3 Hz, 2H, H19), 7.02 (d, J=8.5 Hz, 2H, H14), 5.60-5.49 (m, 3H, H6+7), 4.66-4.61 (m, 1H, H5), 4.37-4.32 (m, 1H, H5), 3.99-3.95 (m, 1H, H9), 3.68 (dd, J=13.5, 4.7 Hz, 1H, H9), 3.52 (s, 3H, H8), 1.98-1.90 (m, 1H, H10), 1.01 (d, J=6.8 Hz, 3H, H11), 0.60 (d, J=6.8 Hz, 3H, H11).

¹³C NMR (176 MHz, CD₃OD): δ 154.19 (C13), 143.49 (C4), 135.82 (C21), 131.71 (C1), 131.65 (C2), 130.56 (C15), 130.44 (C16), 129.35 (C7), 129.02 (C17), 127.12 (C19), 125.95 (C6), 125.81 (C14), 123.84 (C18), 123.15 (C3), 119.26 (C20), 116.19 (C22), 76.62 (C5), 73.37 (C9), 47.74 (C8), 25.53 (C10), 22.77 (C11), 22.01 (C11).

HRMS (ESI-TOF) m/z: [M]⁺ calculated for C₁₄H₂₂N⁺: 204.1752, found 204.1733. [M-H]⁻ calculated for C₂₀H₁₃O₂⁻: 285.0916, found 285.0916.

mp: 146-148° C. (EtOH).

[α]D=+5.14 (MeOH, c=0.5).

XRD: A portion of the ternary complex was crystallised in ethanol, resulting in clear colourless prisms. Crystal data: orthorhombic, space group P212121 (no. 19).

### (R)—N-benzyl-N-(2-methylpropyl)-N-methylbenzenaminium bromide.(R)-1,1′-bi-2-naphthol (2o)

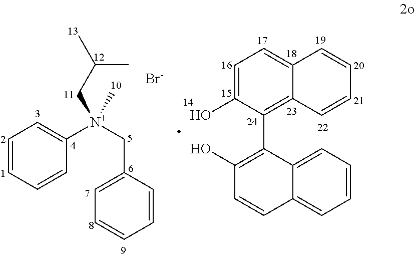 [SMILES: CC(C)C[N@@+](C)(Cc1ccccc1)c1ccccc1 ; [Br-] ; Oc1ccc2ccccc2c1-c1c(O)ccc2ccccc12]

A solution of N-isobutyl-N-methylaniline (0.163 g, 1.0 mmol) was prepared in CHCl₃ (0.66 mL, 1.5 M). Benzyl bromide (0.24 mL, 2.0 mmol) was added with stirring and allowed to react for 10 min. Solid (R)-BINOL (0.286 g, 1.0 mmol) was added to the solution and the reaction vessel sealed and heated to 50° C. After 48 h of heating, the mixture was filtered to isolate 2o as a white solid (0.377 g, 61% yield).

¹H NMR (599 MHz, CD₃OD): δ 7.87 (dd, J=9.0, 0.7 Hz, 2H, H17), 7.83 (d, J=8.1 Hz, 2H, H19), 7.71-7.67 (m, 2H, H3), 7.57 (dd, J=5.2, 2.0 Hz, 3H, H1+2), 7.38 (tt, J=7.6, 1.2 Hz, 1H, H9), 7.30 (d, J=8.9 Hz, 2H, H22), 7.24 (tdd, J=6.7, 3.8, 2.1 Hz, 4H, H7+8), 7.15 (ddd, J=8.2, 6.7, 1.3 Hz, 2H, H21), 7.03 (dd, J=8.5, 1.1 Hz, 2H, H20), 6.96 (d, J=1.5 Hz, 2H, H16), 5.08 (d, J=12.8 Hz, 1H, H5), 4.85 (d, J=12.8 Hz, 1H, H5), 4.24 (dd, J=13.7, 6.0 Hz, 1H, H11), 3.77 (dd, J=13.5, 4.8 Hz, 1H, H11), 3.40 (s, 3H, H10), 1.96-1.85 (m, 1H, H12), 1.06 (d, J=6.7 Hz, 3H, H13), 0.64 (d, J=6.8 Hz, 3H, H13).

¹³C NMR (151 MHz, CD₃OD): δ 154.18 (C15), 142.98 (C4), 135.80 (C23), 133.87 (C7), 131.80 (C1), 131.76 (C9), 131.43 (C2), 130.56 (C17), 130.41 (C18), 129.78 (C8), 129.03 (C19), 128.42 (C6), 127.13 (C21), 125.81 (C16), 123.85 (C3), 123.74 (C20), 119.27 (C22), 116.22 (C24), 76.11 (C5), 75.73 (C11), 46.82 (C10), 25.75 (C12), 22.93 (C13), 22.07 (C13).

HRMS (ESI-TOF) m/z: [M]⁺ calculated for C₁₈H₂₄N⁺: 254.1909, found 254.1898. [M-H]⁻ calculated for C₂₀H₁₃O₂⁻: 285.0916, found 285.0920.

mp: 124° C. (EtOH).

[α]D=−17.89 (MeOH, c=0.5).

XRD: A portion of the ternary complex was crystallised in ethanol, resulting in clear colourless blocks. Crystal data: orthorhombic, space group P212121 (no. 19).

### (S)—N-benzyl-N-isopropyl-N-methylbenzenaminium iodide.(R)-1,1′-bi-2-naphthol (2p)

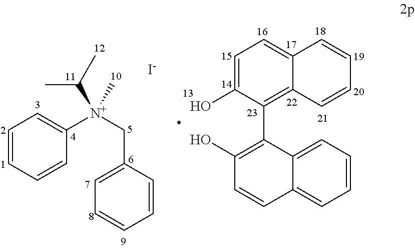 [SMILES: CC(C)[N@@+](C)(Cc1ccccc1)c1ccccc1 ; [I-] ; Oc1ccc2ccccc2c1-c1c(O)ccc2ccccc12]

A solution of N-isopropyl-N-methylaniline (0.149 g, 1.0 mmol) was prepared in MeCN (1.67 mL, 0.6 M). Allyl iodide (0.436 g, 2.0 mmol) was added with stirring and allowed to react for 10 min. Solid (R)-BINOL (0.286 g, 1.0 mmol) was added to the solution and the reaction vessel sealed and heated to 50° C. After 48 h of heating, the mixture was filtered to isolate 2p as a pale yellow solid (0.385 g, 60% yield).

¹H NMR (400 MHz, CD₃OD): δ 7.87 (dd, J=9.0, 0.8 Hz, 2H, H16), 7.85-7.81 (m, 2H, H18), 7.73-7.64 (m, 2H, H3), 7.63-7.53 (m, 3H, H1+2), 7.33 (m, 1H, H9), 7.29 (d, J=8.9 Hz, 2H, H21), 7.27-7.12 (m, 6H, H7+8+19), 7.05-6.97 (m, 4H, H15+20), 5.13 (d, J=12.7 Hz, 1H, H5), 5.00 (d, J=12.7 Hz, 1H, H5), 4.72 (hept, J=6.5 Hz, 1H, H11), 3.26 (s, 3H, H10), 1.75 (d, J=6.4 Hz, 3H, H12), 1.08 (d, J=6.6 Hz, 3H, H12).

¹³C NMR (101 MHz, CD₃OD): δ 154.19 (C14), 143.60 (C4), 135.82 (C22), 133.92 (C7), 131.62 (C1), 131.47 (C2), 131.43 (C9), 130.54 (C16), 130.42 (C17), 129.61 (C8), 129.09 (C3), 129.02 (C18), 127.11 (C20), 125.81 (C15), 123.83 (C19), 123.78 (C6), 119.24 (C21), 116.20 (C23), 72.60 (C5), 70.49 (C11), 40.12 (C10), 17.60 (C12), 17.45 (C12).

HRMS (ESI-TOF) m/z: [M]⁺ calculated for C₁₇H₂₂N⁺: 240.1752, found 240.1752. [M-H]⁻ calculated for C₂₀H₁₃O₂⁻: 285.0916, found 285.0933.

mp: 138-139° C. (EtOH).

[α]D=−47.78 (MeOH, c=0.5).

### (R)—N-benzyl-N-(2-methylpropyl)-N-methylbenzenaminium iodide.(R)-1,1′-bi-2-naphthol (2g)

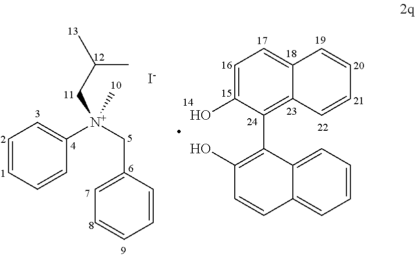 [SMILES: CC(C)C[N@@+](C)(Cc1ccccc1)c1ccccc1 ; [I-] ; Oc1ccc2ccccc2c1-c1c(O)ccc2ccccc12]

A solution of N-isobutyl-N-methylaniline (0.163 g, 1.0 mmol) was prepared in CHCl₃ (0.4 mL, 2.5 M). Benzyl iodide (0.436 g, 2.0 mmol) was added with stirring and allowed to react for 10 min. Solid (R)-BINOL (0.286 g, 1.0 mmol) was added to the solution and the reaction vessel sealed and heated to 50° C. After 48 h of heating, the mixture was filtered to isolate 2q as a white solid (0.277 g, 42% yield).

¹H NMR (400 MHz, CD₃OD): δ 7.87 (dd, J=9.0, 0.8 Hz, 2H, H17), 7.85-7.81 (m, 2H, H19), 7.76-7.67 (m, 2H, H3), 7.63-7.55 (m, 3H, H1+2), 7.44-7.35 (m, 1H, H9), 7.29 (d, J=8.9 Hz, 2H, H22), 7.24 (ddd, J=8.1, 6.7, 1.2 Hz, 4H, H8+20), 7.16 (ddd, J=8.2, 6.8, 1.4 Hz, 2H, H21), 7.06-6.96 (m, 4H, H7+16), 5.11 (d, J=12.7 Hz, 1H, H5), 4.87 (d, J=12.7 Hz, 1H, H5 overlapping with solvent peak), 4.27 (ddd, J=13.6, 6.0, 1.0 Hz, 1H, H11), 3.80 (dd, J=13.5, 4.8 Hz, 1H, H11), 3.44 (s, 3H, H10), 2.00-1.86 (m, 1H, H12), 1.08 (d, J=6.8 Hz, 3H, H13), 0.65 (d, J=6.8 Hz, 3H, H13).

¹³C NMR (101 MHz, CD₃OD): δ 154.18 (C15), 143.00 (C4), 135.81 (C23), 133.89 (C7), 131.84 (C1), 131.81 (C9), 131.45 (C2), 130.55 (C17), 130.42 (C18), 129.81 (C8), 129.02 (C19), 128.43 (C6), 127.11 (C21), 125.80 (C16), 123.83 (C20), 123.78 (C3), 119.24 (C22), 116.19 (C24), 76.08 (C5), 75.69 (C11), 46.82 (C10), 25.78 (C12), 22.95 (C13), 22.08 (C13).

HRMS (ESI-TOF) m/z: [M]⁺ calculated for C₁₈H₂₄N⁺: 254.1909, found 254.1898. [M-H]⁻ calculated for C₂₀H₁₃O₂: 285.0916, found 285.0923.

mp: 128° C. (EtOH).

[α]D=−8.08 (MeOH, c=0.5).

XRD: A portion of the ternary complex was crystallised in ethanol, resulting in clear colourless blocks. Crystal data: orthorhombic, space group P212121 (no. 19).

### Synthesis of Enantio-Enriched Quaternary Ammonium Salts Using (S)-BINOL

### (R)—N-allyl-N-isopropyl-N-methylbenzenaminium bromide.(S)-1,1′-bi-2-naphthol ((ent)-2b)

A solution of N-isopropyl-N-methylaniline (0.298 g, 2.0 mmol) was prepared in CHCl₃ (3.2 mL, 0.6 M). Allyl bromide (0.38 mL, 4.0 mmol) was added with stirring and allowed to react for 10 min. Solid (S)-BINOL (0.572 g, 2.0 mmol) was added to the solution and the reaction vessel sealed and heated to 50° C. After 48 h of heating, the mixture was filtered to isolate (ent)-2b as a white solid (0.878 g, 79% yield).

¹H NMR: spectrum identical to 2b

¹³C NMR: spectrum identical to 2b

mp: 150-152° C. (EtOH).

[α]D=−3.53 (MeOH, c=0.5).

### (R)-(E)-N-(but-2-en-1-yl)-N-isopropyl-N-methylbenzenaminium bromide.(S)-1,1′-bi-2-naphthol ((ent)-2c)

A solution of N-isopropyl-N-methylaniline (0.149 g, 1.0 mmol) was prepared in CHCl₃ (1.67 mL, 0.6 M). Crotyl bromide (0.20 mL, 2.0 mmol) was added with stirring and allowed to react for 10 min. Solid (S)-BINOL (0.286 g, 1.0 mmol) was added to the solution and the reaction vessel sealed and heated to 50° C. After 48 h of heating, the mixture was filtered to isolate (ent)-2c as a white solid (0.360 g, 63% yield).

¹H NMR: spectrum identical to 2c

¹³C NMR: spectrum identical to 2c

mp: 152-153° C. (EtOH).

[α]D=−7.37 (MeOH, c=0.5).

### (R)—N-benzyl-N-isopropyl-N-methylbenzenaminium bromide.(S)-1,1′-bi-2-naphthol ((ent)-2d)

A solution of N-isopropyl-N-methylaniline (0.149 g, 1.0 mmol) was prepared in MeCN (1.67 mL, 0.6 M). Benzyl bromide (0.24 mL, 2.0 mmol) was added with stirring and allowed to react for 10 min. Solid (S)-BINOL (0.286 g, 1.0 mmol) was added to the solution and the reaction vessel sealed and heated to 50° C. After 48 h of heating, the mixture was filtered to isolate (ent)-2d as a white solid (0.450 g, 74% yield).

¹H NMR: spectrum identical to 2d

¹³C NMR: spectrum identical to 2d

mp: 150-151° C. (EtOH).

[α]D=+55.34 (MeOH, c=0.5).

### (R)—N-allyl-N-isopropyl-N-methylbenzenaminium iodide.(S)-1,1′-bi-2-naphthol ((ent)-2k)

A solution of N-isopropyl-N-methylaniline (0.149 g, 1.0 mmol) was prepared in CHCl₃ (1.67 mL, 0.6 M). Allyl iodide (0.18 mL, 2.0 mmol) was added with stirring and allowed to react for 10 min. Solid (S)-BINOL (0.286 g, 1.0 mmol) was added to the solution and the reaction vessel sealed and heated to 50° C. After 48 h of heating, the mixture was filtered to isolate (ent)-2k as a pale yellow solid (0.437 g, 72% yield).

¹H NMR: spectrum identical to 2k

¹³C NMR: spectrum identical to 2k

mp: 146-148° C. (EtOH).

[α]D=−3.16 (MeOH, c=0.5).

### (S)—N-allyl-N-(2-methylpropyl)-N-methylbenzenaminium bromide.(S)-1,1′-bi-2-naphthol ((ent)-2m)

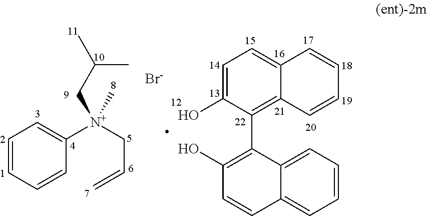 [SMILES: C=CC[N@+](C)(CC(C)C)c1ccccc1 ; [Br-] ; Oc1ccc2ccccc2c1-c1c(O)ccc2ccccc12]

A solution of N-isobutyl-N-methylaniline (0.163 g, 1.0 mmol) was prepared in CHCl₃ (1.67 mL, 0.6 M). Allyl bromide (0.19 mL, 2.0 mmol) was added with stirring and allowed to react for 10 min. Solid (S)-BINOL (0.286 g, 1.0 mmol) was added to the solution and the reaction vessel sealed and heated to 50° C. After 48 h of heating, the mixture was filtered to isolate (ent)-2m as a white solid (0.340 g, 60% yield).

¹H NMR: spectrum identical to 2m

¹³C NMR: spectrum identical to 2m

mp: 147-149° C. (EtOH).

[α]D=−8.55 (MeOH, c=0.5).

XRD: A portion of the ternary complex was crystallised in ethanol, resulting in clear colourless prisms. Crystal data: orthorhombic, space group P212121 (no. 19).

### (S)—N-benzyl-N-(2-methylpropyl)-N-methylbenzenaminium bromide.(S)-1,1′-bi-2-naphthol ((ent)-2o)

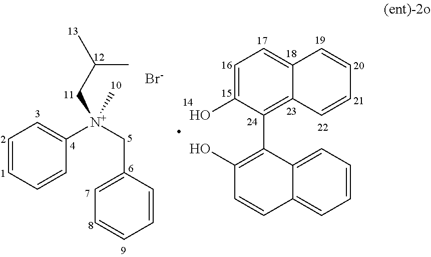 [SMILES: CC(C)C[N@@+](C)(Cc1ccccc1)c1ccccc1 ; [Br-] ; Oc1ccc2ccccc2c1-c1c(O)ccc2ccccc12]

A solution of N-isobutyl-N-methylaniline (0.163 g, 1.0 mmol) was prepared in CHCl₃ (0.66 mL, 1.5 M). Benzyl bromide (0.24 mL, 2.0 mmol) was added with stirring and allowed to react for 10 min. Solid (S)-BINOL (0.286 g, 1.0 mmol) was added to the solution and the reaction vessel sealed and heated to 50° C. After 48 h of heating, the mixture was filtered to isolate (ent)-2o as a white solid (0.388 g, 63% yield).

¹H NMR: spectrum identical to 2o

¹³C NMR: spectrum identical to 2o

mp: 123-125° C. (EtOH).

[α]D=+19.28 (MeOH, c=0.5).

XRD: A portion of the ternary complex was crystallised in ethanol, resulting in clear colourless prisms. Crystal data: orthorhombic, space group P212121 (no. 19).

### (R)—N-benzyl-N-isopropyl-N-methylbenzenaminium iodide.(S)-1,1′-bi-2-naphthol ((ent)-2p)

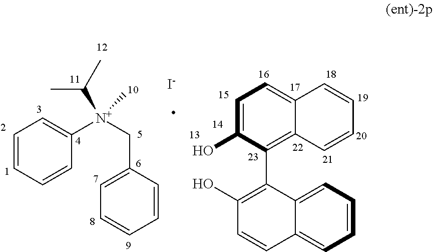

A solution of N-isopropyl-N-methylaniline (0.149 g, 1.0 mmol) was prepared in MeCN (1.67 mL, 0.6 M). Benzyl iodide (0.436 g, 2.0 mmol) was added with stirring and allowed to react for 10 min. Solid (S)-BINOL (0.286 g, 1.0 mmol) was added to the solution and the reaction vessel sealed and heated to 50° C. After 48 h of heating, the mixture was filtered to isolate (ent)-2p as a pale yellow solid (0.346 g, 53% yield).

¹H NMR: spectrum identical to 2p

¹³C NMR: spectrum identical to 2p

mp: 139-140° C. (EtOH).

[α]D=+48.50 (MeOH, c=0.5).

XRD: A portion of the ternary complex was crystallised in ethanol, resulting in clear colourless prisms. Crystal data: orthorhombic, space group P212121 (no. 19).

### (S)—N-benzyl-N-(2-methylpropyl)-N-methylbenzenaminium iodide.(S)-1,1′-bi-2-naphthol ((ent)-2g)

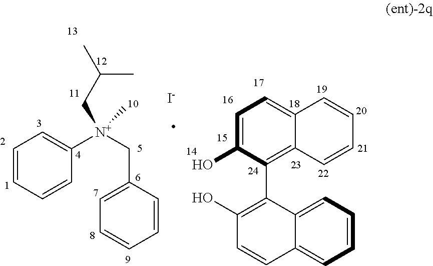

A solution of N-isobutyl-N-methylaniline (0.163 g, 1.0 mmol) was prepared in CHCl₃ (0.40 mL, 2.5 M). Benzyl iodide (0.436 g, 2.0 mmol) was added with stirring and allowed to react for 10 min. Solid (S)-BINOL (0.286 g, 1.0 mmol) was added to the solution and the reaction vessel sealed and heated to 50° C. After 48 h of heating, the mixture was filtered to isolate (ent)-2q as a white solid (0.318 g, 48% yield).

¹H NMR: spectrum identical to 2q

¹³C NMR: spectrum identical to 2q

mp: 128-129° C. (EtOH).

[α]D=+11.00 (MeOH, c=0.5).

XRD: A portion of the ternary complex was crystallised in ethanol, resulting in clear colourless prisms. Crystal data: orthorhombic, space group P212121 (no. 19).

Isolation of Enantio-Enriched Quaternary Ammonium Salts from Ternary Complex

### General Procedure

The solid ternary complex was dissolved into ˜10 mL of MeOH. This solution was then added to a separatory funnel containing 40 mL of deionised H2O and 40 mL of diethyl ether. After separation of the layers, the organic phase was washed with 3×20 mL deionised water. The aqueous phases were then concentrated to dryness under reduced pressure to yield the isolated quaternary ammonium salt.

### (−)-(S)—N-allyl-N-isopropyl-N-methylbenzenaminium bromide ((S)-1b)

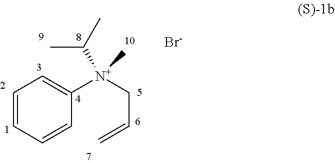

Using 2b (0.269 g, 0.48 mmol) yielded (S)-1b as a white crystalline solid (0.120 g, 99% yield). The organic phase was dried with MgSO₄, filtered, and concentrated to recover pure (R)-BINOL (0.100 g, 73% recovery).

¹H NMR (400 MHz, CD₃OD): δ 7.85-7.78 (m, 2H, H3), 7.67 (t, J=7.9 Hz, 2H, H2), 7.60 (t, J=7.3 Hz, 1H, H1), 5.65-5.56 (m, 1H, H6), 5.52-5.36 (m, 2H, H7), 4.79 (dd, J=13.9, 4.4 Hz, 1H, H5), 4.56-4.39 (m, 2H, H5+8), 3.40 (s, 3H, H10), 1.62 (d, J=6.4 Hz, 3H, H9), 1.09 (d, J=6.6 Hz, 3H, H9).

¹³C NMR (101 MHz, CD₃OD): δ 143.95 (C4), 131.66 (C2), 131.52 (C1), 128.52 (C7), 126.79 (C6), 123.42 (C3), 73.23 (C8), 68.64 (C5), 41.49 (C10), 17.32 (C9), 17.12 (C9).

HRMS (ESI-TOF) m/z: [M]⁺ calculated for C₁₃H₂₀N⁺:190.1590, found: 190.1580.

mp: 140-142° C. (H₂O)

[α]D=−22.06 (MeOH, c=0.5).

### (−)-(S)-(E)-N-(but-2-en-1-yl)-N-isopropyl-N-methylbenzenaminium bromide ((S)-1c)

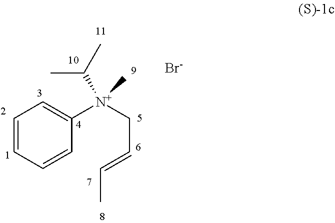

Using 2c (0.246 g, 0.43 mmol) yielded (S)-1c as a white crystalline solid (0.126 g, 99% yield). The organic phase was dried with MgSO₄, filtered, and concentrated to recover pure (R)-BINOL (0.091 g, 75% recovery).

¹H NMR (599 MHz, CD₃OD): δ 7.78 (dd, J=7.8, 1.7 Hz, 2H, H3), 7.66 (dd, J=8.9, 7.1 Hz, 2H, H2), 7.60 (dd, J=8.2, 6.5 Hz, 1H, H1), 6.05 (dq, J=13.5, 6.6 Hz, 1H, H6), 5.07 (dddd, J=15.0, 8.2, 6.2, 1.7 Hz, 1H, H7), 4.67 (dd, J=13.1, 6.1 Hz, 1H, H5), 4.45-4.40 (m, 2H, H5+10), 3.35 (s, 3H, H9), 1.62 (dt, J=6.6, 1.3 Hz, 3H, H8), 1.60 (d, J=6.5 Hz, 3H, H11), 1.08 (d, J=6.6 Hz, 3H, H11).

¹³C NMR (151 MHz, CD₃OD): δ 144.04 (C4), 141.86 (C6), 131.59 (C2), 131.41 (C1), 123.43 (C3), 119.28 (C7), 72.59 (C10), 68.57 (C5), 41.15 (C9), 18.24 (C8), 17.39 (C11), 17.10 (C11).

HRMS (ESI-TOF) m/z: [M]⁺ calculated for C₁₄H₂₂N⁺: 204.1747, found 204.1739.

mp: 151-153° C. (H₂O)

[α]D=−36.30 (MeOH, c=0.5).

### (−)-(S)—N-benzyl-N-isopropyl-N-methylbenzenaminium bromide ((S)-1d)

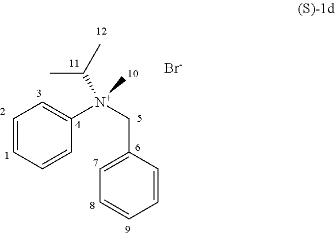 [SMILES: CC(C)[N@+](C)(Cc1ccccc1)c1ccccc1 ; [Br-]]

Using 2d (0.436 g, 0.72 mmol), yielded (S)-1d as a white crystalline solid (0.216 g, 94% yield). The organic phase was dried with MgSO₄, filtered, and concentrated to recover pure (R)-BINOL (0.164 g, 80% recovery).

¹H NMR (700 MHz, CD₃OD): δ 7.77-7.74 (m, 2H, H3), 7.65-7.58 (m, 3H, H1+2), 7.39-7.34 (m, 1H, H9), 7.26-7.22 (m, 2H, H8), 7.07-7.03 (m, 2H, H7), 5.20 (d, J=12.8 Hz, 1H, H5), 5.07 (d, J=12.8 Hz, 1H, H5), 4.79 (hept, J=6.5 Hz, 1H, H11), 3.34 (s, 3H, H10, overlapping with solvent peak) 1.81 (d, J=6.4 Hz, 3H, H12), 1.13 (d, J=6.6 Hz, 3H, H12).

¹³C NMR (176 MHz, CD₃OD): δ 143.68 (C4), 133.93 (C7), 131.65 (C1), 131.49 (C2), 131.45 (C9), 129.63 (C8), 129.15 (C6), 123.83 (C3), 72.65 (C5), 70.57 (C11), 40.18 (C10), 17.62 (C12), 17.45 (C12).

HRMS (ESI-TOF) m/z: [M]⁺ calculated for C₁₇H₂₂N⁺: 240.1747, found: 240.1762.

mp: 110° C. (H₂O).

[α]D=−115.84 (MeOH, c=0.5).

### (−)-(S)—N-allyl-N-isopropyl-N-methylbenzenaminium iodide ((S)-1k)

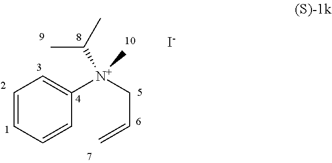

Using 2k (0.210 g, 0.35 mmol) yielded (S)-1k as a white crystalline solid (0.105 g, 95% yield). The organic phase was dried with MgSO₄, filtered, and concentrated to recover pure (R)-BINOL (0.098 g, 98% recovery).

¹H NMR (400 MHz, CD₃OD): δ 7.82 (d, J=8.1 Hz, 2H, H3), 7.67 (t, J=7.8 Hz, 2H, H2), 7.63-7.57 (m, 1H, H1), 5.70-5.54 (m, 1H, H6), 5.52-5.37 (m, 2H, H7), 4.80 (dd, J=13.9, 4.4 Hz, 1H, H5), 4.59-4.39 (m, 2H, H5+8), 3.41 (s, 3H, H10), 1.62 (d, J=6.4 Hz, 3H, H9), 1.10 (d, J=6.5 Hz, 3H, H9).

¹³C NMR (101 MHz, CD₃OD): δ 143.92 (C4), 131.66 (C2), 131.52 (C1), 128.56 (C7), 126.77 (C6), 123.46 (C3), 73.22 (C8), 68.64 (C5), 41.64 (C10), 17.36 (C9), 17.20 (C9).

HRMS (ESI-TOF) m/z: [M]⁺ calculated for C₁₃H₂₀N⁺: 190.1590, found: 190.1585.

mp: 132-133° C. (H₂O).

[α]D=−29.38 (MeOH, c=0.5).

### (−)-(R)—N-allyl-N-(2-methylpropyl)-N-methylbenzenaminium bromide ((R)-1m)

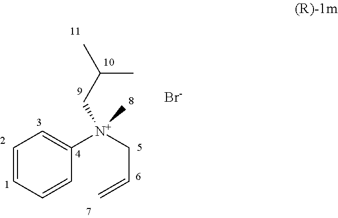

Using 2m (0.319 g, 0.56 mmol) yielded (R)-1m as a white crystalline solid (0.159 g, 89% yield). The organic phase was dried with MgSO₄, filtered, and concentrated to recover pure (R)-BINOL (0.104 g, 65% recovery).

¹H NMR (400 MHz, CD₃OD): δ 7.94-7.82 (m, 2H, H3), 7.76-7.64 (m, 2H, H2), 7.64-7.59 (m, 1H, H1), 5.69-5.49 (m, 3H, H6+7), 4.77-4.64 (m, 1H, H5), 4.43 (ddd, J=13.6, 4.0, 2.3 Hz, 1H, H5), 4.05 (dd, J=13.3, 6.0 Hz, 1H, H9), 3.76 (dd, J=13.4, 4.6 Hz, 1H, H9), 3.60 (s, 3H, H8), 1.98 (m, 1H, H10), 1.04 (d, J=6.8 Hz, 3H, H11), 0.61 (d, J=6.8 Hz, 3H, H11).

¹³C NMR (101 MHz, CD₃OD): δ 143.53 (C4), 131.69 (C1), 131.65 (C2), 129.35 (C7), 126.05 (C6), 123.27 (C3), 76.60 (C5), 73.36 (C9), 47.74 (C8), 25.56 (C10), 22.81 (C11), 22.03 (C11).

HRMS (ESI-TOF) m/z: [M]⁺ calculated for C₁₄H₂₂N⁺: 204.1752, found: 204.1739.

mp: 130-132° C. (H₂O).

[α]D=−10.87 (MeOH, c=0.5).

### (−)-(R)—N-benzyl-N-(2-methylpropyl)-N-methylbenzenaminium bromide ((R)-1o)

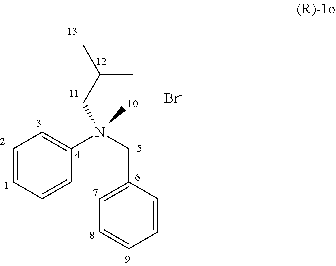 [SMILES: CC(C)C[N@+](C)(Cc1ccccc1)c1ccccc1 ; [Br-]]

Using 20 (0.303 g, 0.49 mmol) yielded (R)-1o as a white crystalline solid (0.145 g, 88% yield). The organic phase was dried with MgSO₄, filtered, and concentrated to recover pure (R)-BINOL (0.090 g, 64% recovery).

¹H NMR (400 MHz, CD₃OD): δ 7.83-7.73 (m, 2H, H3), 7.65 (dd, J=5.3, 2.0 Hz, 3H, H1+2), 7.43 (t, J=7.5 Hz, 1H, H9), 7.30 (t, J=7.7 Hz, 2H, H8), 7.07-7.00 (m, 2H, H7), 5.18 (d, J=12.7 Hz, 1H, H5), 4.94 (d, J=12.6 Hz, 1H, H5), 4.33 (dd, J=13.5, 5.9 Hz, 1H, H11), 3.85 (dd, J=13.5, 4.8 Hz, 1H, H11), 3.52 (s, 3H, H10), 2.11-1.89 (m, 1H, H12), 1.13 (d, J=6.8 Hz, 3H, H13), 0.70 (d, J=6.8 Hz, 3H, H13).

¹³C NMR (101 MHz, CD₃OD): δ 143.10 (C4), 133.91 (C7), 131.87 (C1), 131.85 (C9), 131.48 (C2), 129.84 (C8), 128.51 (C6), 123.83 (C3), 76.15 (C5), 75.77 (C11), 46.83 (C10), 25.81 (C12), 22.95 (C13), 22.09 (C13).

HRMS (ESI-TOF) m/z: [M]⁺ calculated for C₁₈H₂₄N⁺: 254.1909, found 254.1913.

mp: 123-125° C. (H₂O).

[α]D=−56.99 (MeOH, c=0.5).

### (−)-(S)—N-benzyl-N-isopropyl-N-methylbenzenaminium iodide ((S)-1p)

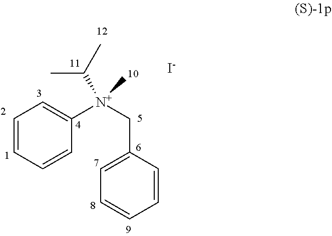 [SMILES: CC(C)[N@+](C)(Cc1ccccc1)c1ccccc1 ; [I-]]

Using 2p (0.270 g, 0.41 mmol) yielded (S)-1p as a white crystalline solid (0.150 g, 95% yield). The organic phase was dried with MgSO₄, filtered, and concentrated to recover pure (R)-BINOL (0.115 g, 98% recovery).

¹H NMR (400 MHz, CD₃OD): δ 7.75 (d, J=6.8 Hz, 2H, H3), 7.68-7.56 (m, 3H, H1+2), 7.36 (t, J=7.5 Hz, 1H, H9), 7.23 (t, J=7.6 Hz, 2H, H8), 7.04 (d, J=8.2 Hz, 2H, H7), 5.20 (d, J=12.7 Hz, 1H, H5), 5.06 (d, J=12.6 Hz, 1H, H5), 4.80 (hept, J=6.4 Hz, 1H, H11), 3.33 (s, 3H, H10, overlapping with solvent peak), 1.80 (d, J=6.3 Hz, 3H, H12), 1.13 (d, J=6.5 Hz, 3H, H12).

¹³C NMR (101 MHz, CD₃OD): δ 143.66 (C4), 133.95 (C7), 131.65 (C1), 131.49 (C2), 131.45 (C9), 129.63 (C8), 129.14 (C6), 123.84 (C3), 72.64 (C5), 70.53 (C11), 40.21 (C10), 17.62 (C12), 17.48 (C12).

HRMS (ESI-TOF) m/z: [M]⁺ calculated for C₁₇H₂₂N⁺: 240.1747, found: 240.1741.

mp: 95-97° C. (H₂O)

[α]D=−98.71 (MeOH, c=0.5).

### (−)-(R)—N-benzyl-N-(2-methylpropyl)-N-methylbenzenaminium iodide ((R)-1q)

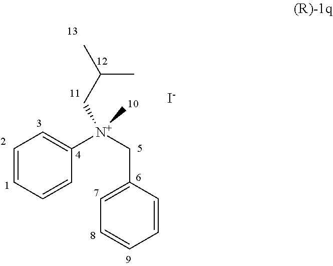 [SMILES: CC(C)C[N@+](C)(Cc1ccccc1)c1ccccc1 ; [I-]]

Using 2q (0.200 g, 0.30 mmol) yielded (R)-1q as a white crystalline solid (0.090 g, 78% yield). The organic phase was dried with MgSO₄, filtered, and concentrated to recover pure (R)-BINOL (0.056 g, 65% recovery).

¹H NMR (400 MHz, CD₃OD): δ 7.83-7.74 (m, 2H, H3), 7.68-7.58 (m, 3H, H1+2), 7.41 (tt, J=7.6, 1.3 Hz, 1H, H9), 7.32-7.23 (m, 2H, H8), 7.07-6.99 (m, 2H, H7), 5.18 (d, J=12.7 Hz, 1H, H5), 4.95 (d, J=12.7 Hz, 1H, H5), 4.33 (ddd, J=13.5, 5.9, 1.0 Hz, 1H, H11), 3.87 (dd, J=13.5, 4.8 Hz, 1H, H11), 3.51 (t, J=0.9 Hz, 3H, H10), 2.06-1.88 (m, 1H, H12), 1.11 (d, J=6.8 Hz, 3H, H13), 0.68 (d, J=6.8 Hz, 3H, H13).

¹³C NMR (101 MHz, CD₃OD): δ 143.09 (C4), 133.93 (C7), 131.83 (C1), 131.81 (C9), 131.46 (C2), 129.81 (C8), 128.54 (C6), 123.86 (C3), 76.12 (C5), 75.70 (C11), 46.83 (C10), 25.81 (C12), 22.96 (C13), 22.10 (C13).

HRMS (ESI-TOF) m/z: [M]⁺ calculated for C₁₈H₂₄N⁺: 254.1909, found 254.1895.

mp: 95-97° C.

[α]D=−43.24 (MeOH, c=0.5).

### (+)-(R)—N-allyl-N-isopropyl-N-methylbenzenaminium bromide ((R)-1b)

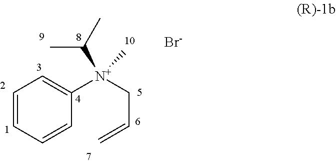

Using (ent)-2b (0.302 g, 0.53 mmol) yielded (R)-1b as a white crystalline solid (0.147 g, 98% yield). The organic phase was dried with MgSO₄, filtered, and concentrated to recover pure (S)-BINOL (0.114 g, 75% recovery).

¹H NMR (400 MHz, CD₃OD): spectrum identical to (S)-1b

¹³C NMR (101 MHz, CD₃OD): spectrum identical to (S)-1b

mp: 139-140° C. (H₂O).

[α]D=+23.09 (MeOH, c=0.5).

### (+)-(R)-(E)-N-(but-2-en-1-yl)-N-isopropyl-N-methylbenzenaminium bromide ((R)-1c)

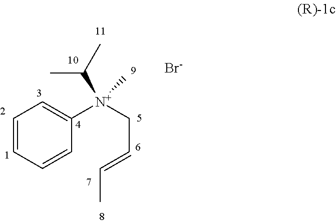

Using (ent)-2c (0.232 g, 0.41 mmol) yielded (R)-1c as a white crystalline solid (0.116 g, 97% yield). The organic phase was dried with MgSO₄, filtered, and concentrated to recover pure (S)-BINOL (0.102 g, 87% recovery).

¹H NMR (400 MHz, CD₃OD): spectrum identical to (S)-1c

¹³C NMR (101 MHz, CD₃OD): spectrum identical to (S)-1c

mp: 150-151° C. (H₂O)

[α]D=+44.14 (MeOH, c=0.5).

### (+)-(R)—N-benzyl-N-isopropyl-N-methylbenzenaminium bromide ((R)-d)

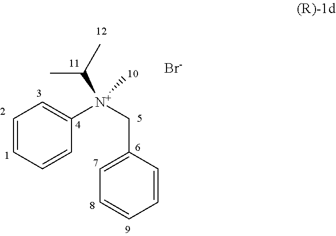 [SMILES: CC(C)[N@@+](C)(Cc1ccccc1)c1ccccc1 ; [Br-]]

Using (ent)-2d (0.265 g, 0.43 mmol) yielded (R)-1d as a white crystalline solid (0.135 g, 96% yield). The organic phase was dried with MgSO₄, filtered, and concentrated to recover pure (S)-BINOL (0.092 g, 75% recovery).

¹H NMR (400 MHz, CD₃OD): spectrum identical to (S)-1d

¹³C NMR (101 MHz, CD₃OD): spectrum identical to (S)-1d

mp: 108-110° C.

[α]D=+122.38 (MeOH, c=0.5).

### (+)-(R)—N-allyl-N-isopropyl-N-methylbenzenaminium iodide ((R)-1k)

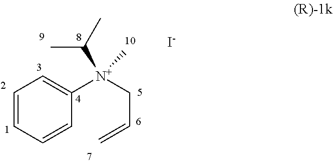

Using (ent)-2k (0.290 g, 0.48 mmol) yielded (R)-1k as a white crystalline solid (0.144 g, 95% yield). The organic phase was dried with MgSO₄, filtered, and concentrated to recover pure (S)-BINOL (0.092 g, 68% recovery).

¹H NMR (400 MHz, CD₃OD): spectrum identical to (S)-1k

¹³C NMR (101 MHz, CD₃OD): spectrum identical to (S)-1k

mp: 130-132° C. (H₂O).

[α]D=+28.28 (MeOH, c=0.5).

### (+)-(S)—N-allyl-N-(2-methylpropyl)-N-methylbenzenaminium bromide ((S)-1m)

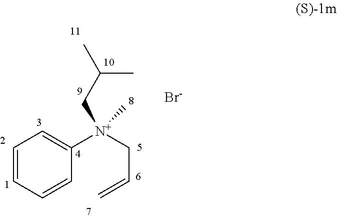

Using (ent)-2m (0.275 g, 0.48 mmol) yielded the desired ammonium salt (S)-1m as a white crystalline solid (0.136 g, 99% yield). The organic phase was dried with MgSO₄, filtered, and concentrated to recover pure (S)-BINOL (0.115 g, 85% recovery).

¹H NMR (400 MHz, CD₃OD): spectrum identical to (R)-1m

¹³C NMR (101 MHz, CD₃OD): spectrum identical to (R)-1m

mp: 132-134° C. (H₂O).

[α]D=+9.04 (MeOH, c=0.5).

### (+)-(S)—N-benzyl-N-(2-methylpropyl)-N-methylbenzenaminium bromide ((S)-1o)

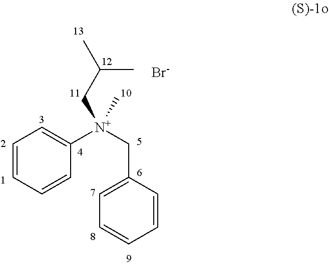 [SMILES: CC(C)C[N@@+](C)(Cc1ccccc1)c1ccccc1 ; [Br-]]

Using (ent)-2o (0.312 g, 0.50 mmol) yielded (S)-1o as a white crystalline solid (0.154 g, 92% yield). The organic phase was dried with MgSO₄, filtered, and concentrated to recover pure (S)-BINOL (0.088 g, 62% recovery).

¹H NMR (400 MHz, CD₃OD): spectrum identical to (R)-1o

¹³C NMR (101 MHz, CD₃OD): spectrum identical to (R)-1o

mp: 125-127° C. (H₂O).

[α]D=+52.12 (MeOH, c=0.5).

### (+)-(R)—N-benzyl-N-isopropyl-N-methylbenzenaminium iodide ((R)-1p)

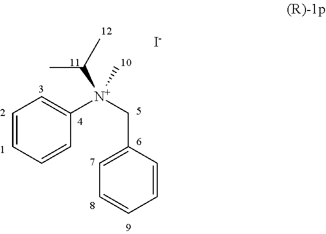 [SMILES: CC(C)[N@@+](C)(Cc1ccccc1)c1ccccc1 ; [I-]]

Using (ent)-2p (0.235 g, 0.36 mmol) yielded (R)-1p as a white crystalline solid (0.124 g, 94% yield). The organic phase was dried with MgSO₄, filtered, and concentrated to recover pure (S)-BINOL (0.070 g, 68% recovery).

¹H NMR (400 MHz, CD₃OD): spectrum identical to (S)-1p

¹³C NMR (101 MHz, CD₃OD): spectrum identical to (S)-1p

mp: 94-96° C. (H₂O).

[α]D=+91.76 (MeOH, c=0.5).

### (+)-(S)—N-benzyl-N-(2-methylpropyl)-N-methylbenzenaminium iodide ((S)-1q)

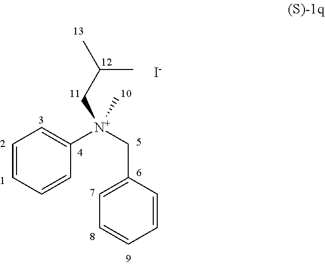 [SMILES: CC(C)C[N@@+](C)(Cc1ccccc1)c1ccccc1 ; [I-]]

Using (ent)-2q (0.202 g, 0.30 mmol) yielded (S)-1q as a white crystalline solid (0.072 g, 63% yield). The organic phase was dried with MgSO₄, filtered, and concentrated to recover pure (S)-BINOL (0.060 g, 70% recovery).

¹H NMR (400 MHz, CD₃OD): spectrum identical to (R)-1q

¹³C NMR (101 MHz, CD₃OD): spectrum identical to (R)-1q

mp: 96-99° C. (H₂O).

[α]D=+53.67 (MeOH, c=0.5).

### Counterion Exchange to Ammonium Hexafluorophosphate Salts

### General Procedure

The quaternary ammonium salt was dissolved into a minimum amount of deionised water. An excess amount of an aqueous saturated solution of KPF₆ was added, resulting in a white precipitate forming. The slurry was extracted with DCM (3×15 mL) and the combined organic layers were dried over MgSO₄. After concentration under reduced pressure, the desired product was acquired.

### (−)-(S)—N-benzyl-N-isopropyl-N-methylbenzenaminium hexafluorophosphate ((S)-1t)

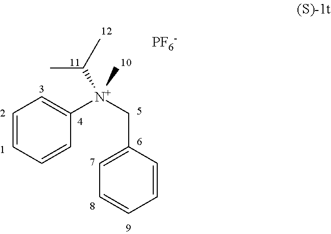

Bromide salt (S)-1d (0.242 g, 0.76 mmol) yielded (S)-1t as a white crystalline solid (0.252 g, 87% yield).

¹H NMR (400 MHz, CD₃CN): δ 7.61-7.49 (m, 5H, H7+8+9), 7.36 (t, J=7.5 Hz, 1H, H1), 7.22 (t, J=7.7 Hz, 2H, H3), 6.96 (d, J=7.7 Hz, 2H, H2), 4.90 (dd, J=29.8, 13.3 Hz, 2H, H5), 4.57 (hept, J=6.6 Hz, 1H, H11), 3.18 (s, 3H, H10), 1.70 (d, J=6.5 Hz, 3H, H12), 1.03 (d, J=6.5 Hz, 3H, H12).

¹³C NMR (101 MHz, CD₃CN): δ 142.96 (C4), 133.70 (C7), 131.48 (C9), 131.31 (C1), 131.27 (C6), 129.51 (C8), 128.58 (C2), 123.41 (C3), 72.51 (C10), 70.29 (C5), 40.27 (C11), 17.58 (C12), 17.33 (C12).

¹⁹F NMR (376 MHz, CD₃CN): δ −72.88 (d, J=706.4 Hz).

³¹P NMR (162 MHz, CD₃CN): δ −144.63 (hept, J=708.9 Hz).

HRMS (ESI-TOF) m/z calculated for C₁₇H₂₂N⁺: 240.1752, found: 240.1741.

mp: 171-172° C. (DCM/MeOH).

[α]D: −75.35 (c=0.5, MeCN).

XRD: crystals were grown from a MeOH/DCM solvent mixture with slow cooling, giving clear colourless prisms. Crystal data: orthorhombic, space group P212121 (no. 19).

### (+)-(R)—N-benzyl-N-isopropyl-N-methylbenzenaminium hexafluorophosphate ((R)-1t)

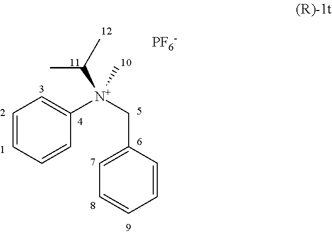

Bromide salt (R)-1d (0.083 g, 0.26 mmol) yielded (R)-1t as a white crystalline solid (0.083 g, 83% yield).

¹H NMR: identical to (S)-1t

¹³C NMR: identical to (S)-1t

¹⁹F NMR: identical to (S)-1t

³¹P NMR: identical to (S)-1t

HRMS (ESI-TOF) m/z calculated for C₁₇H₂₂N⁺: 240.1752, found: 240.1744.

mp: 172-174° C. (DCM/MeOH).

[α]D: +78.11 (c=0.5, MeCN).

XRD: crystals were grown from a MeOH/DCM solvent mixture with slow cooling, giving clear colourless prisms. Crystal data: orthorhombic, space group P212121 (no. 19).

### Recognition of Achiral Ammonium Salts

The following are included not as examples of the method of the invention but in support of the scope of the method of the invention.

### N-allyl-N,N-dimethylbenzenaminium chloride (R)-1,10-bi-2-naphthol (59)

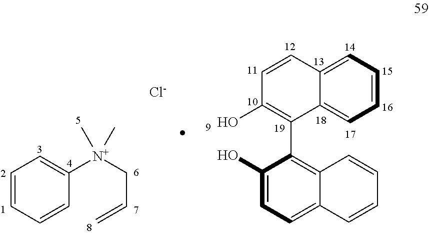

N-allyl-N,N-dimethylbenzenaminium chloride (0.030 g, 0.15 mmol) was dissolved in CHCl₃ (0.4 mL, 0.4 M) in a 10 mL vial. Solid (R)-BINOL (0.043 g, 1.0 equiv) was then added, with stirring, to the solution, resulting in a pale yellow homogenous solution. This solution was stirred at room temperature overnight, which produced 59 as a white precipitate. 59 was isolated by vacuum filtration (0.058 g, 80% yield). Analysis by ¹H NMR spectroscopy confirmed that a 1:1 complex had formed.

¹H NMR (400 MHz, CD₃OD): δ 7.87 (dd, J=9.0, 0.8 Hz, 2H, H12), 7.83 (dt, J=8.2, 0.9 Hz, 2H, H14), 7.81-7.77 (m, 2H, H3), 7.66-7.54 (m, 3H, H1+2), 7.30 (d, J=8.9 Hz, 2H, H17), 7.24 (ddd, J=8.1, 6.8, 1.3 Hz, 2H, H15), 7.16 (ddd, J=8.3, 6.8, 1.3 Hz, 2H, H16), 7.05-6.99 (m, 2H, H11), 5.66-5.47 (m, 3H, H7+8), 4.45 (d, J=5.7 Hz, 2H, H6), 3.55 (s, 6H, H5).

¹³C NMR (101 MHz, CD₃OD): δ 154.19, 145.98, 135.82, 131.64, 130.56, 130.42, 129.33, 129.03, 127.13, 126.29, 125.81, 123.85, 121.99, 119.26, 116.22, 72.64, 54.25.

LRMS (ESI-TOF, EI+) m/z: 162.22 ([M]⁺, 100%).

LRMS (ESI-TOF, EI−) m/z: 285.26 ([M-H]⁻, 100%).

HRMS (ESI-TOF) m/z: [M]⁺ calculated for C₁₁H₁₆N⁺: 162.1283, found 162.1276. [M-H]⁻ calculated for C₂₀H₁₃O₂⁻: 285.0921, found 285.0897.

mp: 148° C., decomposes to red oil.

IR (nmax/cm⁻¹): 3048br, 2982m, 1622m, 1505m, 1430m, 1339m, 1273m, 952w, 819m, 757m, 690m.

XRD: A portion of the complex was crystallised in ethanol, resulting in clear colourless prisms. Crystal data for C₃₀H₂₈NO₂Cl (m=469.98 g/mol): Orthorhombic, space group P 212121 (no. 19).

### N-allyl-N,N-dimethylbenzenaminium iodide (R)-1,10-bi-2-naphthol (61)

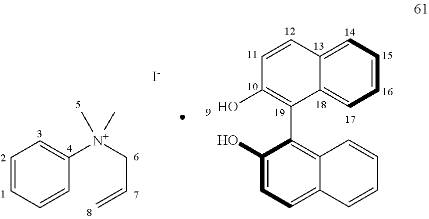

N-allyl-N,N-dimethylbenzenaminium iodide (0.144 g, 1.00 mmol) was dissolved in CHCl₃ (1.25 mL, 0.8 M) in a 10 mL vial. Solid (R)-BINOL (0.286 g, 1.0 equiv) was then added, with stirring, to the solution, resulting in a pale red homogenous solution. This solution was allowed to stir at room temperature overnight, which produced 61 as a white precipitate. 61 was isolated by vacuum filtration (0.229 g, 80% yield). Analysis by ¹H NMR spectroscopy confirmed that a 1:1 complex had formed.

¹H NMR (400 MHz, CD₃OD): δ 7.87 (dd, J=9.0, 0.7 Hz, 2H, H12+14), 7.85-7.81 (m, 4H, H3+2), 7.70-7.59 (m, 1H, H1), 7.29 (d, J=8.9 Hz, 2H, H17), 7.24 (ddd, J=8.0, 6.7, 1.3 Hz, 2H, H15), 7.16 (ddd, J=8.3, 6.8, 1.4 Hz, 2H, H16), 7.02 (dd, J=8.5, 1.1 Hz, 2H, H11), 5.72-5.52 (m, 3H, H7+8), 4.51 (d, J=6.2 Hz, 2H, H6), 3.61 (s, 6H, H5).

¹³C NMR (101 MHz, CD₃OD): δ 154.17, 145.95, 135.80, 131.63, 130.56, 130.41, 129.38, 129.03, 127.13, 126.28, 125.79, 123.84, 122.04, 119.25, 116.20, 72.62, 54.36.

LRMS (ESI-TOF, EI+) m/z: 162.19 ([M]⁺, 100%), 130.31 (2), 110.10 (6).

LRMS (ESI-TOF, EI−) m/z: 285.23 ([M-H]⁻, 100%).

HRMS (ESI-TOF) m/z: [M]⁺ calculated for C₁₁H₁₆N⁺: 162.1283, found 162.1278. [M-H]⁻ calculated for C₂₀H₁₃O₂⁻: 285.0921, found 285.0900.

mp: 154° C.

IR (nmax/cm⁻¹): 3185br, 1621m, 1505m, 1317m, 1145m, 952m, 815s, 753m, 679m.

XRD: Complex was crystallised in ethanol, resulting in clear colourless plates. Crystal data for C₃₁H₃₀INO₂ (m=575.46 g/mol): Orthorhombic, space group P 212121 (no. 19).

### N,N-dimethyl-N-(prop-2-yn-1-yl)benzenaminium bromide (R)-1,10-bi-2-naphthol (71)

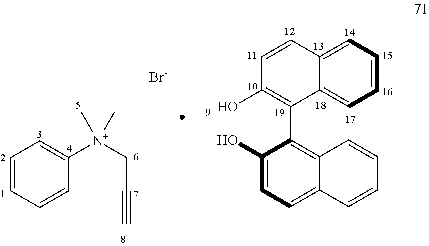

N,N-dimethyl-N-(prop-2-yn-1-yl)benzenaminium bromide (0.155 g, 1.0 mmol) was dissolved in CHCl₃ (1.6 mL, 0.4 M) in a 10 mL vial. Solid (R)-BINOL (0.645 g, 1.0 equiv) was then added, with stirring, to the solution, resulting in a pale yellow homogenous solution. This solution was allowed to stir at room temperature overnight, which produced 71 as a white precipitate. 71 was isolated by vacuum filtration (0.307 g, 90% yield). Analysis by ¹H NMR spectroscopy confirmed that a 1:1 complex had formed.

¹H NMR (400 MHz, CD₃OD): δ 7.90-7.85 (m, 4H, H12+14), 7.83 (dt, J=8.1, 1.0 Hz, 2H, H3), 7.69-7.57 (m, 3H, H1+2), 7.29 (d, J=8.9 Hz, 2H, H17), 7.24 (ddd, J=8.1, 6.8, 1.3 Hz, 2H, H15), 7.16 (ddd, J=8.2, 6.8, 1.4 Hz, 2H, H16), 7.05-6.99 (m, 2H, H11), 4.87 (d, J=2.6 Hz, 2H, H6), 3.69 (s, 6H5), 3.36 (t, J=2.5 Hz, 1H, H8).

¹³C NMR (101 MHz, CD₃OD): δ 154.18, 146.28, 135.81, 131.91, 131.61, 130.56, 130.43, 129.02, 127.12, 125.80, 123.84, 121.90, 119.25, 116.18, 82.89, 72.67, 60.02, 54.79.

LRMS (ESI-TOF) m/z: 160.55 ([M]⁺, 100%), 121.07 (3).

LRMS (ESI-TOF) m/z: 285.67 ([M]⁺, 100%).

HRMS (ESI-TOF) m/z: [M]⁺ calculated for C₁₁H₁₄N⁺: 160.1126, found 160.1129. [M-H]⁻ calculated for C₂₀H₁₃O₂⁻: 285.0921, found 285.0895.

mp: 149-150° C., decomposes to red oil.

IR (nmax/cm⁻¹): 3141br, 2124w, 1620m, 1505m, 1336w, 1273m, 970w, 816m, 752m.

XRD: A portion of the complex was crystallised in ethanol, resulting in clear colourless plates. Crystal data for C₃₁H₂₇BrNO₂C₂H₅OH (m=571.51 g/mol): Orthorhombic, space group P 212121 (no. 19).

### 1-allyl-1,4-diazabicyclo[2.2.2]octan-1-ium bromide (R)-1,10-bi-2-naphthol (108)

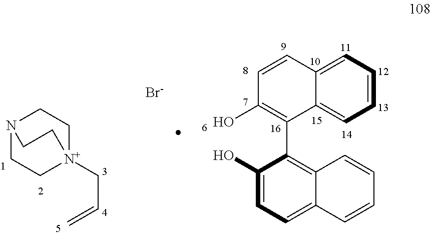

1-allyl-1,4-diazabicyclo[2.2.2]octan-1-ium bromide (0.100 g, 0.43 mmol) was dissolved in EtOH (1.0 mL, 0.4 M) in a 10 mL vial. Solid (R)-BINOL (0.122 g, 1.0 equiv) was then added, with stirring, to the solution, resulting in a pale yellow homogenous solution. This solution was stirred at room temperature overnight, which produced 108 as a white precipitate. 108 was isolated by vacuum filtration (0.142 g, 64% yield).

¹H NMR (400 MHz, DMSO-d₆): δ 9.23 (s, 2H, H6), 7.90-7.81 (m, 4H, H9+11), 7.35 (d, J=8.8 Hz, 2H, H14), 7.23 (ddd, J=8.0, 6.7, 1.3 Hz, 2H, H12), 7.16 (ddd, J=8.2, 6.7, 1.4 Hz, 2H, H13), 7.00-6.88 (m, 2H, H8), 6.00 (ddt, J=16.3, 10.7, 7.3 Hz, 1H, H4), 5.66-5.54 (m, 2H, H5), 3.92 (d, J=7.5 Hz, 2H, H3), 3.0-3.20 (m, 12H, H1+2).

¹³C NMR (101 MHz, DMSO-d₆): δ 152.98, 134.08, 128.59, 128.08, 127.83, 127.26, 125.79, 125.42, 124.37, 122.23, 118.52, 115.37, 65.14, 51.56, 44.63.

LRMS (ESI-TOF, EI+) m/z: 153.22 ([M]⁺, 100%).

LRMS (ESI-TOF, EI−) m/z: 285.27 ([M-H]⁻, 100%).

HRMS (ESI-TOF) m/z: [M]⁺ calculated for C₉H₁₂N₂⁺: 153.1392, found 153.1396. [M-H]⁻ calculated for C₂₀H₁₃O₂⁻: 285.0921, found 285.0901.

mp: 180° C.

IR (nmax/cm⁻¹): 3185br, 1624m, 1506m, 1433m, 1338m, 1272s, 1054m, 811s, 749m, 638m, 439w.

XRD: A portion of the complex was crystallised in ethanol, resulting in clear colourless prisms. Crystal data for C₂₉H₂₉BrNO₂C₂H₅OH (m=549.52 g/mol): Orthorhombic, space group P 212121 (no. 19).

### 1-benzyl-1,4-diazabicyclo[2.2.2]octan-1-ium bromide (R)-1,10-bi-2-naphthol (110)

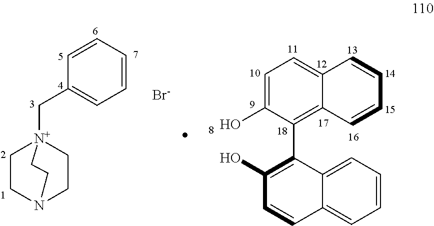

1-benzyl-1,4-diazabicyclo[2.2.2]octan-1-ium bromide (0.283 g, 1.0 mmol) was dissolved in CHCl₃ (1.67 mL, 0.6 M) in a 10 mL vial. Solid (R)-BINOL (0.286 g, 1.0 equiv.) was then added, with stirring, to the solution. This solution was stirred at room temperature overnight, which produced 110 as a white precipitate. 110 was isolated by vacuum filtration (0.527 g, 93% yield).

¹H NMR (400 MHz, DMSO-d₆): δ 9.23 (s, 2H, H7), 7.89-7.81 (m, 4H, H11+13), 7.52 (d, J=2.4 Hz, 5H, H5-7), 7.32 (d, J=8.9 Hz, 2H, H16), 7.23 (ddd, J=8.1, 6.8, 1.3 Hz, 2H, H14), 7.16 (ddd, J=8.3, 6.8, 1.5 Hz, 2H, H15), 6.93 (dd, J=8.3, 1.2 Hz, 2H, H10), 4.52 (s, 2H, H3), 3.29 (t, J=7.5 Hz, 6H, H2), 3.01 (dd, J=8.9, 6.1 Hz, 6H, H1).

¹³C NMR (101 MHz, DMSO-d₆) δ 153.45, 134.56, 133.67, 130.66, 129.46, 129.09, 128.56, 128.32, 127.65, 126.30, 124.85, 122.73, 118.99, 115.85, 66.92, 52.03, 45.11.

HRMS (ESI-TOF) m/z: [M]⁺ calculated for C₁₃H₁₉N₂⁺: 203.1548, found: 203.1539. [M-H]⁻ calculated for C₂₀H₁₃O₂⁻: 285.0921, found: 285.0917.

mp: 222-223° C. (EtOH).

IR (nmax/cm⁻¹): 3190br, 1623m, 1503m, 1336m, 1270m, 816m, 745m.

XRD: Crystallised in EtOH, to give clear colourless prisms. Crystal data for C₃₃H₃₄BrN₂O₂ (m=570.53 g/mol): Orthorhombic, space group P 212121 (no. 19).

### 1-benzyl-1-methyl-morpholinium bromide (R)-1,10-bi-2-naphthol (114)

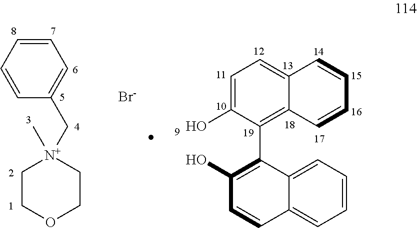

1-benzyl-1-methyl-morpholinium bromide (0.272 g, 1.0 mmol) was dissolved in CHCl₃ (1.67 mL, 0.6 M) in a 10 mL vial. Solid (R)-BINOL (0.286 g, 1.0 equiv) was then added, with stirring, to the solution. This solution was stirred at room temperature overnight, which produced 114 as a white precipitate. 114 was isolated by vacuum filtration (0.600 g, 81% yield).

¹H NMR (400 MHz, DMSO-d₆) δ 9.22 (s, 2H, H9), 7.88-7.81 (m, 4H, H12+14), 7.61-7.47 (m, 5H, H6-8), 7.32 (d, J=8.8 Hz, 2H, H17), 7.23 (ddd, J=8.1, 6.7, 1.3 Hz, 2H, H15), 7.16 (ddd, J=8.3, 6.7, 1.5 Hz, 2H, H16), 6.93 (dd, J=8.3, 1.2 Hz, 2H, H11), 4.71 (s, 2H, H4), 4.08-3.88 (m, 4H, H2), 3.53 (ddd, J=13.1, 8.7, 4.4 Hz, 2H, H1), 3.31 (dt, J=13.2, 2.9 Hz, 2H, H10), 3.06 (s, 3H, H3).

¹³C NMR (101 MHz, DMSO-d₆) δ 153.00, 134.12, 133.25, 130.42, 128.97, 128.65, 128.12, 127.88, 127.19, 125.86, 124.41, 122.29, 118.55, 115.40, 59.88, 58.56.

HRMS (ESI-TOF) m/z: [M]⁺ calculated C₁₂H₁₈NO⁺: 192.1388, found: 192.1373. [M-H]⁻ calculated for C₂₀H₁₃O₂⁻: 285.0921, found: 285.0932.

mp: 212-214° C. (EtOH).

IR (nmax/cm⁻¹): 3245br, 2980w, 1620m, 1274m, 1270m, 1124m, 751m.

XRD: Crystallised in EtOH, to give clear colourless blocks. Crystal data for C₃₂H₃₂BrNO₃ (m=558.49 g/mol): Monoclinic, space group P 21 (no. 4).

### N-benzyl-N-methylpyrollidinium bromide (R)-1,10-bi-2-naphthol (112)

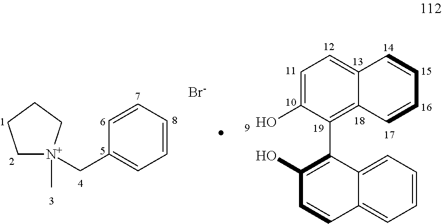

N-benzyl-N-methylpyrollidinium bromide (0.256 g, 1.0 mmol) was dissolved in CHCl₃ (1.67 mL, 0.6 M) in a 10 mL vial. Solid (R)-BINOL (0.286 g, 1.0 equiv) was then added, with stirring, to the solution. This solution was stirred at room temperature overnight, which produced 112 as a white precipitate. 112 was isolated by vacuum filtration (0.484 g, 89% yield). Analysis by ¹H NMR spectroscopy confirmed that a 1:1 complex had formed.

¹H NMR (400 MHz, DMSO-d₆) δ 9.23 (s, 2H, H9), 7.85 (dd, J=8.6, 4.4 Hz, 4H, H12+14), 7.70-7.46 (m, 5H, H6-8), 7.34 (d, J=8.9 Hz, 2H, H17), 7.23 (t, J=7.0 Hz, 2H, H15), 7.16 (ddd, J=8.2, 6.7, 1.4 Hz, 2H, H16), 6.94 (d, J=8.4 Hz, 2H, H11), 4.61 (s, 2H, H4), 3.58 (q, J=8.9, 6.8 Hz, 2H, H2), 3.44-3.33 (m, 2H, H20), 2.90 (s, 3H, H3), 2.27-2.02 (m, 4H, H1).

¹³C NMR (101 MHz, DMSO-d₆): δ 153.01, 134.11, 132.52, 130.17, 129.09, 128.96, 128.63, 128.11, 127.87, 125.84, 124.40, 122.27, 118.55, 115.40, 65.03, 62.64, 47.18, 20.77.

HRMS (ESI-TOF) m/z: [M]⁺ calculated for C₁₂H₁₈N⁺: 176.1439, found: 176.1433. [M-H]⁻ calculated for C₂₀H₁₃O₂⁻: 285.0921, found 285.0937.

mp: 197-198° C. (EtOH).

IR (nmax/cm⁻¹): 3141br, 1622m, 1504m, 1271m, 811m, 748m.

XRD: crystallised in EtOH, to give clear colourless blocks. Crystal data for C₃₁H₃₂NO₂Br (m=530.48 g/mol): Monoclinic, space group P 21 (no. 4).

### N-benzyl-N,N-dimethyl-2-oxo-2-phenylethan-1-aminium bromide (R)-1,10-bi-2-naphthol (82)

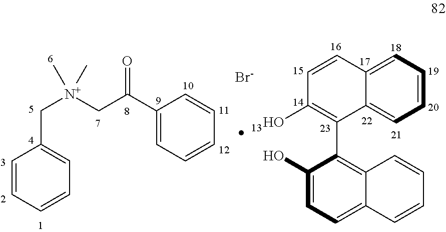

N-benzyl-N,N-dimethyl-2-oxo-2-phenylethan-1-aminium bromide (0.100 g, 0.30 mmol) was dissolved in EtOH (0.38 mL, 0.8 M) in a 1 mL vial. Solid (R)-BINOL (0.085 g, 1.0 equiv) was then added, with stirring, to the solution, resulting in a pale yellow homogenous solution. This solution was stirred at room temperature for 15 minutes, which produced 82 as a white precipitate. 82 was isolated by vacuum filtration (0.139 g, 77% yield). Analysis by ¹H NMR spectroscopy confirmed that a 1:1 complex had formed.

¹H NMR (400 MHz, CDCl₃): δ 8.00 (d, J=7.6 Hz, 2H, H16), 7.91 (d, J=8.9 Hz, 2H, H18), 7.86 (d, J=8.1 Hz, 2H, H3), 7.58 (t, J=7.3 Hz, 1H, H1), 7.50-7.44 (m, 9H, H2+10−12+21), 7.34 (t, J=7.4 Hz, 2H, H19), 7.26 (dd, J=16.1, 8.1 Hz, 2H20), 7.14 (d, J=8.4 Hz, 2H15), 6.12 (s, 2H, H13), 5.44 (t, J=14.8 Hz, 2H, H7), 4.98 (s, 2H, H5), 3.30 (s, 6H, H6).

¹³C NMR (101 MHz, CDCl₃): δ 191.31, 152.75, 135.01, 134.08, 133.66, 131.03, 130.80, 129.42, 129.24, 129.20, 128.52, 128.29, 127.16, 126.84, 124.49, 123.73, 118.07, 112.07, 68.22, 66.21, 50.87.

XRD: crystallised in MeCN, to give clear colourless needles. Crystal data for C₃₇H₃₄NO₃Br (m=620.56 g/mol): orthorhombic, space group P 212121 (no. 19).

### N-allyl-N,N-dimethylbenzenaminium acetate (R)-1,10-bi-2-naphthol (63)

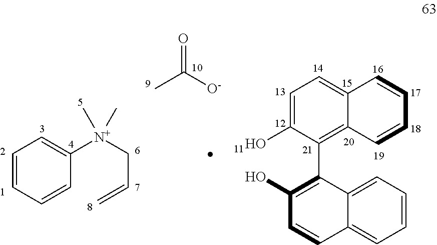

N-allyl-N,N-dimethylbenzenaminium acetate (0.221 g, 1.0 mmol) was dissolved in CHCl₃ (1.67 mL, 0.6 M) in a 10 mL vial. Solid (R)-BINOL (0.286 g, 1.0 equiv) was then added, with stirring, to the solution. This solution was stirred at room temperature overnight, which produced 63 as a white precipitate. 63 was isolated by vacuum filtration (0.338 g, 67% yield). Analysis by ¹H NMR spectroscopy confirmed that a 1:1 complex had formed.

¹H NMR (400 MHz, DMSO-d₆) δ 7.93-7.85 (m, 2H, H3), 7.82-7.72 (m, 4H, H14+16), 7.62 (ddd, J=8.1, 6.9, 1.6 Hz, 2H, H2), 7.59-7.51 (m, 1H, H1), 7.32 (d, J=8.8 Hz, 2H, H19), 7.14 (ddd, J=8.0, 6.7, 1.4 Hz, 2H, H17), 7.08 (ddd, J=8.2, 6.7, 1.5 Hz, 2H, H18), 6.93 (dd, J=8.4, 1.3 Hz, 2H, H13), 5.58 (ddt, J=17.2, 9.2, 6.9 Hz, 1H, H7), 5.50-5.39 (m, 2H, H8), 4.52 (d, J=7.0 Hz, 2H, H6), 3.55 (s, 6H, H5), 1.68 (s, 3H, H9).

¹³C NMR (101 MHz, DMSO-d₆) δ 173.96, 156.06, 145.12, 134.76, 130.51, 128.48, 128.23, 128.18, 127.96, 126.27, 125.60, 125.22, 121.79, 121.72, 120.52, 116.63, 70.73, 53.61, 25.12.

HRMS (ESI-TOF) m/z: [M]⁺ calculated for C₁₁H₁₆N⁺: 162.1283, found 162.1275. [M-H]⁻ calculated for C₂₀H₁₃O₂: 285.0921, found: 285.0928.

mp: 189-191° C. (EtOH).

Supramolecular Recognition of BINOL with Pseudoenantiomeric Cinchona Derived Ammonium Salts

### N-Benzylcinchonidinium Chloride.(R)-BINOL

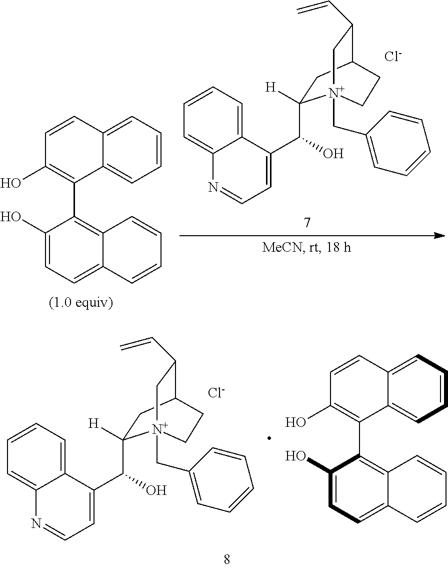

Racemic BINOL (0.286 g, 1.0 mmol) was dissolved with stirring into MeCN (3.8 mL, 0.26 M). Solid N-benzylcinchonidinium chloride 7 (0.231 g, 0.55 mmol) was added to the solution. After approximately 1 mins, complexation was observed. The solution was allowed to stir at room temperature for 18 h. The resulting precipitate was isolated by vacuum filtration to give ternary complex 8 as a white solid (0.309 g, 88% yield).

¹H NMR (599 MHz, DMSO-d₆) δ 9.22 (2H, s), 8.98 (1H, d, J=4.5 Hz), 8.28 (1H, dd, J=8.6, 1.3 Hz), 8.11 (1H, dd, J=8.5, 1.3 Hz), 7.84 (5H, ddd, J=8.3, 4.2, 2.7 Hz), 7.81 (1H, d, J=4.4 Hz), 7.77-7.70 (3H, m), 7.57 (3H, ddt, J=5.2, 3.7, 2.1 Hz), 7.33 (2H, d, J=8.9 Hz), 7.23 (2H, ddd, J=8.0, 6.7, 1.2 Hz), 7.16 (2H, ddd, J=8.2, 6.7, 1.4 Hz), 6.93 (2H, dd, J=8.5, 1.2 Hz), 6.55 (1H, s), 5.68 (1H, ddd, J=17.2, 10.6, 6.6 Hz), 5.28 (1H, d, J=12.3 Hz), 5.14 (1H, dt, J=17.3, 1.3 Hz), 4.96 (2H, t, J=11.4, 10.2 Hz), 4.39-4.29 (1H, m), 3.94-3.88 (1H, m), 3.71 (1H, ddd, J=12.7, 5.2, 3.0 Hz), 3.34-3.28 (4H, m), 3.20 (1H, td, J=11.3, 5.1 Hz), 2.75-2.63 (1H, m), 2.17-2.03 (2H, m), 1.85-1.77 (1H, m), 1.30 (1H, td, J=11.8, 10.1, 3.4 Hz).

¹³C NMR (151 MHz, DMSO-d₆) δ 153.0, 150.2, 147.6, 145.3, 138.1, 134.1, 133.8, 130.1, 129.9, 129.4, 128.9, 128.6, 128.1, 128.0, 127.8, 127.2, 125.8, 124.4, 124.3, 123.6, 122.2, 120.1, 118.5, 116.3, 115.4, 67.8, 63.9, 62.7, 59.3, 50.5, 36.8, 25.9, 24.2, 20.9.

HRMS (ESI-TOF) m/z: [M]⁺ Calculated for C₂₆H₂₉N₂O⁺: 385.2274, found: 385.2278.

HRMS (ESI-TOF) m/z: [M-H]⁻ Calculated for C₂₀H₁₃O₂⁻: 285.0921, found: 285.0919.

XRD: sample was crystallised in ethanol. Crystal data for C₄₆H₄₅ClN₂O₄: orthorhombic, P2₁2₁2₁.

### N-Benzylcinchoninium Chloride.(R)-BINOL

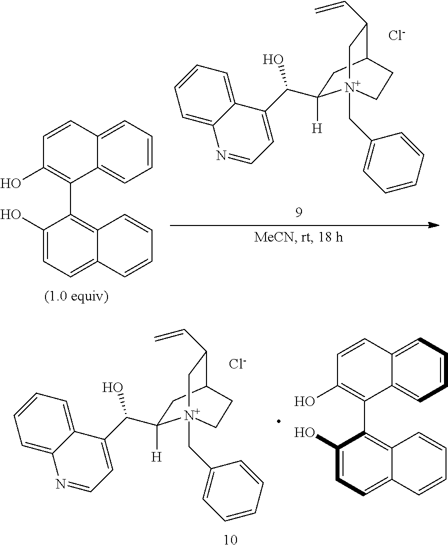

Racemic BINOL (0.286 g, 1.0 mmol) was dissolved with stirring into MeCN (3.8 mL, 0.26 M). Solid N-benzylcinchoninium chloride (0.231 g, 0.55 mmol) was added to the solution. After approximately 1 min, complexation was observed. The solution was allowed to stir at room temperature for 18 h. The resulting precipitate was isolated by vacuum filtration to give ternary complex 10 as a white solid (0.388 g, 100% yield).

¹H NMR (599 MHz, CD₃OD) δ 9.25 (2H, s), 8.98 (1H, d, J=4.4 Hz), 8.34 (1H, dd, J=8.5, 1.3 Hz), 8.11 (1H, dd, J=8.4, 1.3 Hz), 7.87-7.81 (6H, m), 7.80-7.71 (3H, m), 7.59-7.55 (3H, m), 7.35 (2H, d, J=8.9 Hz), 7.22 (2H, ddd, J=8.1, 6.7, 1.3 Hz), 7.16 (2H, ddd, J=8.2, 6.7, 1.4 Hz), 6.93 (2H, dd, J=8.5, 1.1 Hz), 6.53-6.48 (1H, m), 6.01 (1H, ddd, J=17.3, 10.6, 7.0 Hz), 5.23 (1H, dt, J=4.9, 1.4 Hz), 5.21 (1H, dt, J=11.4, 1.4 Hz), 5.16 (1H, d, J=12.4 Hz), 5.09 (1H, d, J=12.4 Hz), 4.27 (1H, ddd, J=11.9, 8.4, 2.7 Hz), 3.99-3.84 (2H, m), 3.49-3.40 (1H, m), 3.01-2.89 (1H, m), 2.63 (1H, q, J=8.9 Hz), 2.33-2.27 (1H, m), 1.89-1.82 (1H, m), 1.79-1.69 (2H, m), 1.09-1.00 (1H, m).

¹³C NMR (151 MHz, CD₃OD) δ 153.0, 150.2, 147.6, 145.2, 137.1, 134.1, 133.8, 130.1, 129.8, 129.4, 128.9, 128.5, 128.1, 128.0, 127.8, 127.2, 125.8, 124.4 (2×C), 123.8, 122.2, 120.1, 118.6, 117.0, 115.4, 67.3, 64.4, 62.2, 56.0, 53.6, 36.7, 26.3, 23.0, 20.7.

NB: The apparent missing carbon resonance is due to a coincident CH and quaternary carbon signal at 124.4 ppm, as confirmed by HMBC.

HRMS (ESI-TOF) m/z: [M]⁺ Calculated for C₂₆H₂₉N₂O⁺: 385.2274, found: 385.2280.

HRMS (ESI-TOF) m/z: [M-H]⁻ Calculated for C₂₀H₁₃O₂⁻: 285.0921, found: 285.0924.

XRD: sample crystallised in ethanol. Crystal data for C46H45ClN2O4: orthorhombic, P2₁2₁2₁.

Supramolecular Recognition of BINOL with Enantiomeric Ammonium Salts

### (S)—N-benzyl-N-isopropyl-N-methylbenzenaminium bromide.(R)-1,1′-bi-2-naphthol

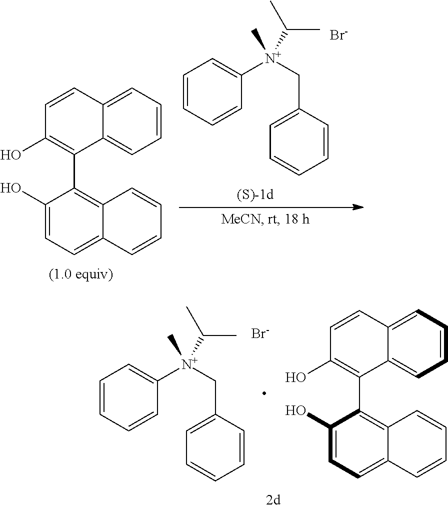

Racemic BINOL (0.286 g, 1.0 mmol) was dissolved into MeCN (1.6 mL, 0.6 M) with stirring. Solid (S)-1d (0.160 g, 0.5 mmol) was added to the solution. The solution was allowed to stir at room temperature for 18 h. The resulting precipitate was isolated by vacuum filtration to give a white solid (0.206 g, 68% yield).

¹H NMR (599 MHz, CD₃OD) spectrum identical to 2d—previously described kinetic resolution.

¹³C NMR (151 MHz, CD₃OD) spectrum identical to 2d—previously described kinetic resolution.

HRMS (ESI-TOF) m/z: [M]⁺ Calculated for C₁₇H₂₂N⁺: 240.1747, found: 240.1750.

HRMS (ESI-TOF) m/z: [M-H]⁻ Calculated for C₂₀H₁₃O₂⁻: 285.0921, found: 285.0925.

### (R)—N-benzyl-N-isopropyl-N-methylbenzenaminium bromide.(S)-1,1′-bi-2-naphthol

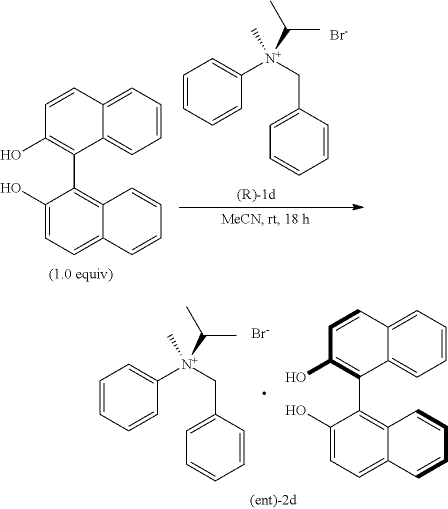

Racemic BINOL (0.286 q, 1.0 mmol) was dissolved into MeCN (1.6 mL, 0.6 M) with stirring. Solid (R)-1d (0.160 q, 0.5 mmol) was added to the solution. The solution was allowed to stir at room temperature for 18 h. The resulting precipitate was isolated by vacuum filtration to give a white solid (0.196 q, 65% yield).

¹H NMR (599 MHz, CD₃OD) spectrum identical to (ent)-2d—previously described kinetic resolution.

¹³C NMR (151 MHz, CD₃OD) spectrum identical to (ent)-2d—previously described kinetic resolution.

HRMS (ESI-TOF) m/z: [M]⁺ Calculated for C₁₇H₂₂N⁺: 240.1747, found: 240.1744.

HRMS (ESI-TOF) m/z: [M-H]⁻ Calculated for C₂₀H₁₃O₂⁻: 285.0921, found: 285.0918.

Recovery of Enriched BINOL from Ternary Complexes

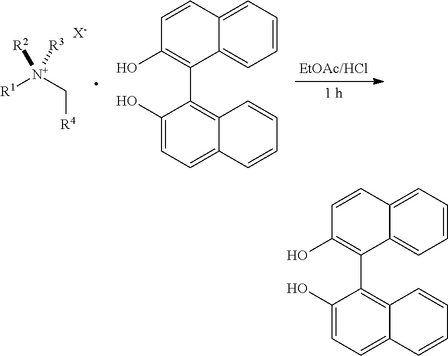

Recovery from Ternary Complex 8—(R)-BINOL

Using complex 8 (0.150 g, 0.21 mmol), the solid was suspended into EtOAc (3 mL), dilute HCl (1.6 mL, 5% v/v) and deionised water (1.4 mL). This solution was then stirred vigorously for 1 hour. The solution was then transferred to a separatory funnel and separated. The aqueous layer was further extracted with EtOAc (3×3 mL). The combined organic phases were dried over MgSO₄ and concentrated under reduced pressure to yield the enriched (R)-BINOL species as a white crystalline solid (0.059 g, 97% yield). The solid was further azeotroped with ethanol to remove traces of acetic acid from hydrolysed EtOAc. Enantioenrichment of the bulk material was analysed by a chiral HPLC assay and was calculated to be 92:8 er (R:S).

¹H NMR (400 MHz, CDCl₃) δ 7.96 (2H, d, J=9.0 Hz), 7.90 (2H, dd, J=8.0, 1.4 Hz), 7.44-7.35 (4H, m), 7.31 (2H, ddd, J=8.2, 6.8, 1.4 Hz), 7.16 (2H, dd, J=8.4, 1.2 Hz), 5.06 (2H, s).

¹³C NMR (101 MHz, CDCl₃) δ 152.9, 133.5, 131.5, 129.6, 128.5, 127.6, 124.3, 124.2, 117.9, 110.9.

HRMS (ESI-TOF) m/z: [M-H]⁻ Calculated for C₂₀H₁₃O₂⁻: 285.0921, found: 285.0918.

Chiral HPLC method: The enantioenrichment of the material was measured using a DAICELAS-H column (100 mm), using 90% hexanes 10% iPrOH mobile phase set at a 1 mL·min-1 flowrate. Peak elution was monitored by UV-PDA detector (A=254 nm). S enantiomer Rt=12.5 min, R enantiomer Rt=18.0 min. Each injection was given a full runtime of 40 mins. Peak identification was achieved by comparison to known standards.

Recovery from Ternary Complex 10—(R)-BINOL

Using complex 10 (0.150 g, 0.21 mmol), the solid was suspended into EtOAc (3 mL), dilute HCl (1.6 mL, 5% v/v) and deionised water (1.4 mL). This solution was then stirred vigorously for 1 hour. The solution was then transferred to a separatory funnel and separated. The aqueous layer was further extracted with EtOAc (3×3 mL). The combined organic phases were dried over MgSO₄ and concentrated under reduced pressure to yield the enriched (R)-BINOL species as a white crystalline solid (0.058 g, 96% yield). The solid was further azeotroped with ethanol to remove traces of acetic acid from hydrolysed EtOAc. Enantioenrichment of the bulk material was analysed by a chiral HPLC assay and was calculated to be 85:15 er (R:S).

¹H NMR (400 MHz, CDCl₃) δ spectrum identical to material recovered from complex 8

¹³C NMR (101 MHz, CDCl₃) δ spectrum identical to material recovered from complex 8

Chiral HPLC method: The enantioenrichment of the material was measured using a DAICELAS-H column (100 mm), using 90% hexanes 10% iPrOH mobile phase set at a 1 mL·min-1 flowrate. Peak elution was monitored by UV-PDA detector (A=254 nm). S enantiomer Rt=12.5 min, R enantiomer Rt=18.0 min. Each injection was given a full runtime of 40 mins. Peak identification was achieved by comparison to known standards.

Recovery from Ternary Complex 2d—(R)-BINOL

Using complex 2d (0.150 g, 0.25 mmol), the solid was suspended into EtOAc (3 mL), dilute HCl (1.6 mL, 5% v/v) and deionised water (1.4 mL). This solution was then stirred vigorously for 1 hour. The solution was then transferred to a separatory funnel and separated. The aqueous layer was further extracted with EtOAc (3×3 mL). The combined organic phases were dried over MgSO₄ and concentrated under reduced pressure to yield the enriched (R)-BINOL species as a white crystalline solid (0.070 g, 99% yield). The solid was further azeotroped with ethanol to remove traces of acetic acid from hydrolysed EtOAc. Enantioenrichment of the bulk material was analysed by a chiral HPLC assay and was calculated to be 97:3 er (R:S).

¹H NMR (400 MHz, CDCl₃) δ spectrum identical to material recovered from complex 8

¹³C NMR (101 MHz, CDCl₃) δ spectrum identical to material recovered from complex 8

Chiral HPLC method: The enantioenrichment of the material was measured using a DAICELAS-H column (100 mm), using 90% hexanes 10% iPrOH mobile phase set at a 1 mL·min-1 flowrate. Peak elution was monitored by UV-PDA detector (A=254 nm). S enantiomer Rt=12.5 min, R enantiomer Rt=18.0 min. Each injection was given a full runtime of 40 mins. Peak identification was achieved by comparison to known standards.

Recovery from Ternary Complex (Ent)-2d—(S)-BINOL

Using complex (ent)-2d (0.150 g, 0.25 mmol), the solid was suspended into EtOAc (3 mL), dilute HCl (1.6 mL, 5% v/v) and deionised water (1.4 mL). This solution was then stirred vigorously for 1 hour. The solution was then transferred to a separatory funnel and separated. The aqueous layer was further extracted with EtOAc (3×3 mL). The combined organic phases were dried over MgSO₄ and concentrated under reduced pressure to yield the enriched (S)-BINOL species as a white crystalline solid (0.070 g, 99% yield). The solid was further azeotroped with ethanol to remove traces of acetic acid from hydrolysed EtOAc. Enantioenrichment of the bulk material was analysed by a chiral HPLC assay and was calculated to be >1:99 er (R:S).

¹H NMR (400 MHz, CDCl₃) δ spectrum identical to material recovered from complex 8

¹³C NMR (101 MHz, CDCl₃) δ spectrum identical to material recovered from complex 8

Chiral HPLC method: The enantioenrichment of the material was measured using a DAICELAS-H column (100 mm), using 90% hexanes 10% iPrOH mobile phase set at a 1 mL·min-1 flowrate. Peak elution was monitored by UV-PDA detector (A=254 nm). S enantiomer Rt=12.5 min, R enantiomer Rt=18.0 min. Each injection was given a full runtime of 40 mins. Peak identification was achieved by comparison to known standards.

1. A method of making an enantiomerically enriched tertiary or quaternary ammonium salt comprising reacting a tertiary amine with a compound of formula R—X to form a tertiary or quaternary ammonium salt, wherein the tertiary amine is chiral at the nitrogen atom, R is different to any substituent on the nitrogen atom of the tertiary amine and X is a leaving group and wherein the reacting is effected under reversible conditions in the presence of a non-racemic chiral compound having at least two substituents capable of coordinating to the tertiary or quaternary ammonium salt.

2. The method of claim 1, wherein the ratio of tertiary amine to R—X is any one selected from the group consisting of 1:>1, 1:≥1.2, 1:≥1.4, 1:≥1.6, 1:≥1.8 and 1:≥2.

3. (canceled)

4. The method of claim 1, wherein the ratio of tertiary amine to non-racemic chiral compound is any one selected from the group consisting of 1:>0.5, 1:≥0.6, 1:≥0.7, 1:≥0.8, 1:≥0.9 and 1:≥1.

5. (canceled)

6. The method of claim 1, wherein the tertiary amine is of formula N(R¹)₃, wherein each R¹ is a different hydrocarbyl group optionally comprising one or more heteroatoms selected from the group consisting of oxygen, nitrogen, sulphur, fluorine, boron, bromine, chlorine, phosphorous and iodine.

7. The method of claim 1, wherein R is a hydrocarbyl group which is different to each R¹.

8. The method of claim 1, wherein the tertiary amine is of formula N(R¹)₃, and each R¹ is independently selected from the group consisting of C₁-C₆alkyl, C₂-C₆alkenyl, C₂-C₆alkynyl, C₆-C₁₀aryl, C₆-C₂₄biaryl, C₆-C₁₀arylC₁-C₆alkyl, C₆-C₂₄biarylC₁-C₆alkyl, C₆-C₁₀arylacyl, C₆-C₂₄biarylacyl, C₃-C₈cycloalkyl, C₃-C₈cycloalkylC₁-C₆alkyl and C₃-C₅heteroaryl, optionally substituted with any one or a combination selected from the group consisting of hydroxy, oxo and amino; or
two R¹ groups together with the nitrogen atom to which they are attached form indolyl, tetrahydroquinolinyl, 3-azabicyclo[3.2.1]octanyl or camphidinyl, optionally substituted with any one or a combination selected from the group consisting of C₁-C₆alkyl, C₂-C₆alkenyl, C₂-C₆alkynyl, hydroxy, oxo and amino; morpholino, pyrrolidino or piperidinyl, substituted with any one or a combination selected from the group consisting of C₁-C₆alkyl, C₂-C₆alkenyl, C₂-C₆alkynyl, hydroxy, oxo and amino such that the resultant N(R¹)₂ is asymmetric; and the other R¹ group is selected from the group consisting of C₁-C₆alkyl, C₂-C₆alkenyl, C₂-C₆alkynyl, C₆-C₁₀aryl, C₆-C₂₄biaryl, C₆-C₁₀arylC₁-C₆alkyl, C₆-C₂₄biarylC₁-C₆alkyl, C₆-C₁₀arylacyl, C₆-C₂₄biarylacyl, C₃-C₈cycloalkyl and C₃-C₈cycloalkylC₁-C₆alkyl, optionally substituted one or more times with any one or a combination selected from the group consisting of hydroxy, oxo and amino; or
all three R¹ groups together with the nitrogen atom to which they are attached form 1,4-diazabicyclo[2.2.2]octane or 1-azabicyclo[2.2.2]octane substituted with any one or a combination selected from the group consisting of C₁-C₆alkyl, C₂-C₆alkenyl, C₂-C₆alkynyl, hydroxy, oxo and amino such that the resultant N(R¹)₃ is chiral; or
N(R¹)₃ is morphine, nalorphine, naltrexone, oxymorphone, or atropine.

9. The method of claim 1, wherein the tertiary amine is of formula N(R¹)₃, and each R¹ is independently selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted with any one or a combination selected from the group consisting of hydroxy, oxo and amino; or
two R¹ groups together with the nitrogen atom to which they are attached form indolyl, tetrahydroquinolinyl, 3-azabicyclo[3.2.1]octanyl or camphidinyl, optionally substituted with any one or a combination selected from the group consisting of C₁-C₆alkyl, C₂-C₆alkenyl, C₂-C₆alkynyl, hydroxy, oxo and amino; morpholino, pyrrolidino or piperidinyl, substituted with any one or a combination selected from the group consisting of C₁-C₆alkyl, C₂-C₆alkenyl, C₂-C₆alkynyl, hydroxy, oxo and amino such that the resultant N(R¹)₂ is asymmetric; and the other R¹ is selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted one or more times with any one or a combination selected from the group consisting of hydroxy, oxo and amino; or
all three R¹ groups together with the nitrogen atom to which they are attached form 1,4-diazabicyclo[2.2.2]octane or 1-azabicyclo[2.2.2]octane substituted with any one or a combination selected from the group consisting of C₁-C₆alkyl, C₂-C₆alkenyl, C₂-C₆alkynyl, hydroxy, oxo and amino such that the resultant N(R¹)₃ is chiral; or
N(R¹)₃ is morphine, nalorphine, naltrexone, oxymorphone, or atropine.

10. The method of claim 1, wherein the tertiary amine is of formula N(R¹)₃, and each R¹ is independently selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted with one or more hydroxy and/or amino; or a
two R¹ groups together with the nitrogen atom to which they are attached form indolyl, tetrahydroquinolinyl, 3-azabicyclo[3.2.1]octanyl or camphidinyl, optionally substituted with any one or a combination selected from the group consisting of C₁-C₆alkyl, C₂-C₆alkenyl, C₂-C₆alkynyl, hydroxy and amino; and the other R¹ group is selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl, optionally substituted with one or more hydroxy and/or amino; or
N(R¹)₃ is morphine, nalorphine, naltrexone, oxymorphone, or atropine.

11. The method of claim 1, wherein the tertiary amine is of formula N(R¹)₃, and each R¹ is independently selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl; or
two R¹ groups together with the nitrogen atom to which they are attached form indolyl, tetrahydroquinolinyl, 3-azabicyclo[3.2.1]octanyl or camphidinyl, optionally substituted with any one or a combination selected from the group consisting of C₁-C₆alkyl, C₂-C₆alkenyl, C₂-C₆alkynyl, hydroxy and amino; and the other R¹ group is selected from the group consisting of C₁-C₆alkyl, C₂-C₆alkenyl, C₂-C₆alkynyl, C₆-C₁₀aryl, C₆-C₁₀arylC₁-C₆alkyl, C₆-C₁₀arylacyl, C₃-C₈cycloalkyl and C₃-C₈cycloalkylC₁-C₆alkyl; or
N(R¹)₃ is morphine, nalorphine, naltrexone, oxymorphone, or atropine.

12. The method of claim 1, wherein R is selected from the group consisting of C₁-C₆alkyl, C₂-C₆alkenyl, C₂-C₆alkynyl, C₆-C₁₀aryl, C₆-C₂₄biaryl, C₆-C₁₀arylC₁-C₆alkyl, C₆-C₂₄biarylC₁-C₆alkyl and C₃-C₈cycloalkyl.

13. (canceled)

14. The method of claim 8, wherein C₁-C₆alkyl is C₁-C₄alkyl, C₂-C₆alkenyl is C₂-C₄alkenyl, C₂-C₆alkynyl is C₂-C₄alkynyl, C₆-C₁₀aryl is phenyl, C₆-C₁₀arylC₁-C₆alkyl is phenylC₁-C₆alkyl and phenylC₁-C₆alkyl is benzyl.

15. The method of claim 1, wherein the tertiary amine has three substituents each of which is unconnected to the other two substituents and each has a different Taft steric substituent constant (Eₛ) and the Taft steric substituent constants differ by >0.07.

16. The method of claim 1, wherein X is selected from the group consisting of halo, triflate, tosylate, phosphate and acetoxy.

17. (canceled)

18. The method of claim 1, wherein the at least two substituents capable of coordinating to the tertiary or quaternary ammonium salt are each —OH.

19. The method of claim 1, wherein the chiral compound has two substituents capable of coordinating to the tertiary or quaternary ammonium salt.

20. The method of claim 1, wherein the chiral compound is any one of structures (I) to (III): 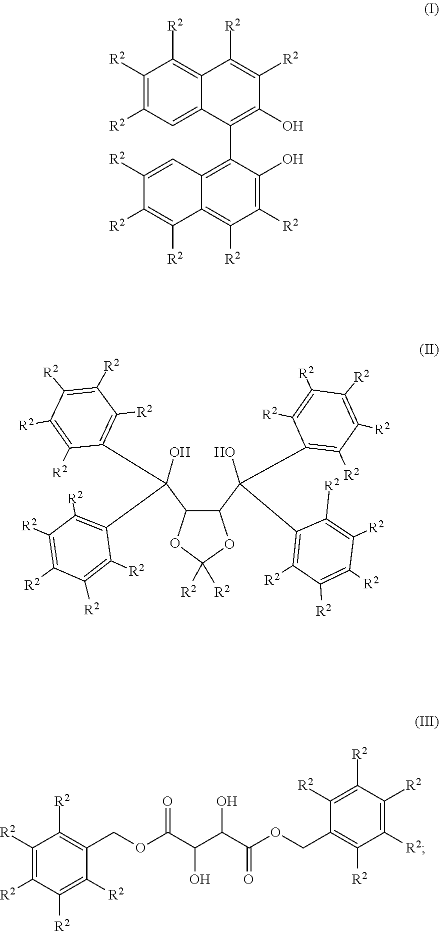
wherein each R² is independently selected from the group consisting of —H, C₁-C₆alkyl, C₂-C₆alkenyl, C₂-C₆alkynyl, C₆-C₁₀aryl, C₆-C₁₀arylC₁-C₆alkyl, C₁-C₆mono-, di- or tri-alkylC₆-C₁₀aryl, C₁-C₆mono-, di- or tri-fluoroalkylC₆-C₁₀aryl, tri-C₆-C₁₀arylsilyl, C₃-C₈cycloalkyl, C₃-C₈cycloalkylC₁-C₆alkyl, C₁-C₆mono-, di- or tri-alkylC₃-C₈cycloalkyl, C₁-C₆mono-, di- or tri-fluoroalkylC₃-C₈cycloalkyl.

21. (canceled)

22. (canceled)

23. The method of claim 20, wherein each R² is —H.

24. The method of claim 1, wherein the chiral compound is an atropisomeric biaryl compound.

25. The method of claim 1, wherein the chiral compound is [1,1′-binaphthalene]-2,2′-diol.

26. The method of claim 1, further comprising isolating the tertiary or quaternary ammonium salt as a ternary complex comprising a tertiary or quaternary ammonium cation, anion X⁻ and chiral compound.

27. The method of claim 26, further comprising recrystallizing the ternary complex to form a recrystallised ternary complex.

28. The method of claim 26, further comprising isolating the tertiary or quaternary ammonium salt as an isolated tertiary or quaternary ammonium salt comprising a tertiary or quaternary ammonium cation and an anion X⁻.

29. The method of claim 28, further comprising exchanging anion X⁻ for a different anion selected from the group consisting of [PF₆]⁻, [BF₄]⁻, [ClO₄]⁻, [B(C₆F₅)₄]⁻, [B(3,5-(CF₃)₂C₆H₃)₄]⁻, —OTf, F⁻, Cl⁻, Br⁻, I⁻, ⁻OH, ⁻OTs, ⁻OAc, [H₂PO₄]⁻, [HSO₄]⁻ and [CH₃SO₃]⁻.

30.-32. (canceled)
